(19) United States
(12) Patent Application Publication
(10) Pub. No.: US 2024/0271143 A1
(43) Pub. Date: Aug. 15, 2024

(54) POLYNUCLEIC ACID MOLECULES TARGETING PCSK9 AND USES THEREOF

(71) Applicant: Sirius Therapeutics, Inc., San Diego, CA (US)

(72) Inventor: Curt BRADSHAW, San Diego, CA (US)

(21) Appl. No.: 18/614,396

(22) Filed: Mar. 22, 2024

Related U.S. Application Data
(63) Continuation of application No. PCT/US2022/044444, filed on Sep. 22, 2022.
(60) Provisional application No. 63/337,958, filed on May 3, 2022.
(60) Provisional application No. 63/247,677, filed on Sep. 23, 2021.

(51) Int. Cl. C12N 15/113

(52) U.S. Cl. CPC C12N 15/1137; C12N 2310/14; C12N 2310/315; C12N 2310/321; C12N 2310/322; C12N 2310/351

(57) ABSTRACT

Disclosed herein are siRNA duplexes, pharmaceutical compositions, and methods for suppressing the expression of proprotein convertase subtilisin/kexin type 9 serine protease (PCSK9).

### CROSS-REFERENCE TO RELATED APPLICATIONS

This application is a continuation of International Application No. PCT/US2022/044444, which was internationally filed on Sep. 22, 2022, which claims the benefit of U.S. Provisional Application No. 63/247,677 filed on Sep. 23, 2021, and U.S. Provisional Application No. 63/337,958 filed on May 3, 2022, all of which are incorporated herein by reference in their entireties.

### SEQUENCE LISTING

The instant application contains a Sequence Listing which has been submitted electronically in XML format and is hereby incorporated by reference in its entirety. Said XML copy, created on Mar. 21, 2024, is named 61382-708-301-SL.xml and is 3,207,920 bytes in size.

### BACKGROUND OF THE DISCLOSURE

The discovery of RNA interference (RNAi) as a cellular mechanism that selectively degrades mRNAs allows for both the targeted manipulation of cellular phenotypes in cell culture and the potential for development of directed therapeutics (Behlike, 2006, Mol, Ther. 13, 644-670; Xie et al., 2006, Drug Discov. Today I1, 67-73). As another RNA therapeutics, antisense oligonucleotides (ASOs) is involved in RNA processing and modulates protein expression (Rinaldi & Wood, 2018, Nature Reviews Neurology volume 14, 9-21).

Proprotein convertase subtilisin/kexin type 9 serine protease (PCSK9) gene is the ninth member of the mammalian family of serine proteinases. PCSK9 plays an important role in the metabolism of low-density lipoproteins (LDL), development and progression of the neurological diseases including neuroinflammation, Alzheimer's Disease, alcohol use disorder (AUD), stroke, etc. Accordingly, there is a need for developing an effective PCSK9 inhibitor without cytotoxicity. The polynucleic acid molecules, conjugates thereof, and methods described herein satisfy this need and provide related advantages.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

Disclosed herein, in certain aspects, are polynucleic acid molecules for modulating expression of PCSK9 gene, wherein the polynucleic acid molecule comprises a nucleic acid sequence in Table 1, Table 2, and Table 3.

In some aspects, the polynucleic acid molecule is a single-stranded nucleic acid molecule. In some instances, the single-stranded nucleic acid molecule comprises at least 14, 15, 16, 17, 18 consecutive nucleotides that are complementary to a sequence selected from SEQ ID NOs: 1, 3, 5, 7, 9, 25-127, and 437-539, with no more than 1, 2, 3, 4 mismatches. In other instances, the single-stranded nucleic acid molecule comprises a sequence that is at least 80%, at least 85%, at least 90%, at least 95% complementary to a sequence selected from SEQ ID NOs: 1, 3, 5, 7, 9, 25-127, and 437-539. In some instances, the single-stranded nucleic acid molecule comprises at least 14, 15, 16, 17, 18 consecutive nucleotides that are complementary to a sequence selected from SEQ ID NOs: 1, 3, 5, 7, and 9, with no more than 1, 2, 3, 4 mismatches. In other instances, the single-stranded nucleic acid molecule comprises a sequence that is at least 80%, at least 85%, at least 90%, at least 95% complementary to a sequence selected from SEQ ID NOs: 1, 3, 5, 7, and 9.

In some aspects, the polynucleic acid molecule is a double-stranded nucleic acid molecule comprising a sense strand and an antisense strand. In some instances, the sense strand comprises at least 80%, at least 85%, at least 90%, at least 95% identical to a sequence selected from SEQ ID NOs: 1, 3, 5, 7, 9, 25-127, and 437-539. In some instances, the antisense strand comprises at least 80%, at least 85%, at least 90%, at least 95% identical to a sequence selected from SEQ ID NOs: 2, 4, 6, 8, 10, and 231-333. In some instances, the sense strand comprises a nucleic acid sequence comprising at least 14, 15, 16, 17, 18, 19, or 20 consecutive nucleotides of a sequence selected from SEQ ID NOs: 1, 3, 5, 7, 9, 25-127, and 437-539 with no more than 1, 2, 3, or 4 mismatches. In other instances, the antisense strand comprises a nucleic acid sequence comprising at least 14, 15, 16, 17, 18, 19, or 20 consecutive nucleotides of a sequence selected from SEQ ID NOs: 2, 4, 6, 8, 10, and 231-333 with no more than 1, 2, 3, or 4 mismatches. In some instances, the sense strand comprises one of SEQ ID NOs: 1, 3, 5, 7, 9, 25-127, and 437-539 and the antisense strand comprises one of SEQ ID NOs: 2, 4, 6, 8, 10, and 231-333. In some instances, the sense strand comprises at least 80%, at least 85%, at least 90%, at least 95% identical to a sequence selected from SEQ ID NOs: 1, 3, 5, 7, and 9. In some instances, the antisense strand comprises at least 80%, at least 85%, at least 90%, at least 95% identical to a sequence selected from SEQ ID NOs: 2, 4, 6, 8, and 10. In some instances, the sense strand comprises a nucleic acid sequence comprising at least 14, 15, 16, 17, 18, 19, or 20 consecutive nucleotides of a sequence selected from SEQ ID NOs: 1, 3, 5, 7, and 9 with no more than 1, 2, 3, or 4 mismatches. In other instances, the antisense strand comprises a nucleic acid sequence comprising at least 14, 15, 16, 17, 18, 19, or 20 consecutive nucleotides of a sequence selected from SEQ ID NOs: 2, 4, 6, 8, and 10 with no more than 1, 2, 3, or 4 mismatches. In some instances, the sense strand comprises one of SEQ ID NOs: 1, 3, 5, 7, 9, 25-127, and 437-539 and the antisense strand comprises one of SEQ ID NOs: 2, 4, 6, 8, and 10.

In some instances, the sense strand comprises at least 90%, at least 95% identical to a sequence selected from SEQ ID NOs: 1, 3, 5, 7, and 9. In some instances, the antisense strand comprises at least 90%, at least 95% identical to a sequence selected from SEQ ID NOs: 2, 4, 6, 8, and 10. In other instances, the sense strand comprises a nucleic acid sequence comprising at least 14, 15, 16, 17, 18, 19, or 20 consecutive nucleotides of a sequence selected from SEQ ID NOs: 1, 3, 5, 7, 9 with no more than 1, 2, 3, or 4 mismatches. In other instances, the antisense strand comprises a nucleic acid sequence comprising at least 14, 15, 16, 17, 18, 19, or 20 consecutive nucleotides of a sequence selected from SEQ ID NOs: 2, 4, 6, 8, and 10 with no more than 1, 2, 3, or 4 mismatches. In other instances, the sense strand comprises one of SEQ ID NOs: 1, 3, 5, 7, and 9 and the antisense strand comprises one of 2, 4, 6, 8, and 10.

In some aspects, the polynucleic acid molecule comprises (1) a 2′-fluoro modified nucleotides; (2) a 2′-O-methyl modified nucleotides; or (3) a modified internucleotide linkage. In some instances, the polynucleic acid molecule comprises at least two consecutive modified internucleotide linkages at the 5′ end. In some instances, the polynucleic acid molecule comprises at least two internucleotide linkages among three internucleotide linkages at the 3′end substituted with modified internucleotide linkages.

In some instances, the sense strand comprises ‘5-NfsnsNfnNfnNfnNfNfNfnNfnNfnNfnNfnNf-3’, wherein the antisense strand comprises ‘5-nsNfsnNfnNfnNfnNfnnnNfnNfnNfnNfnsnsn-3’, wherein “Nf” stands for a 2′-fluoro modified nucleotide, “n” stands for a 2′-O-methyl modified nucleotide, “s” stands for a 3′-phosphorothioate. In other instances, the sense strand comprises ‘5-nsnsnnnnNfnNfNfNfnnnnnnnnnn-3’, wherein the antisense strand comprises ‘5-nsNfsnnnNfnNfNfnnnnNfnNfnnnnnsnsn-3’, wherein “Nf” stands for a 2′-fluoro modified nucleotide, “n” stands for a 2′-O-methyl modified nucleotide, “s” stands for a 3′-phosphorothioate. In other instances, the sense strand comprises ‘5-nsnsnnnnnnNfnNfnnnnnnnnnn-3’, wherein the antisense strand comprises ‘5-nsNfsnnnnnnnnnNfnNfhnnnnnnsnsn-3’, wherein “Nf” stands for a 2′-fluoro modified nucleotide, “n” stands for a 2′-O-methyl modified nucleotide, “s” stands for a 3′-phosphorothioate. In other instances, the sense strand comprises ‘5-nsnsnnnnNfnNfnNfnnnnnnnnnn-3’, wherein the antisense strand comprises ‘5-nsNfsnnnnnnnnnNfnNfnNfnnnnnsnsn-3’, wherein “Nf” stands for a 2′-fluoro modified nucleotide, “n” stands for a 2′-O-methyl modified nucleotide, “s” stands for a 3′-phosphorothioate.

In some instances, the modified internucleotide linkage is a phosphorothioate linkage. In some instances, the modified internucleotide linkage comprises a stereochemically enriched phosphorothioate internucleotide linkage. In some instances, the modified internucleotide linkage is an SP chiral internucleotide phosphorothioate linkage. In some instances, the polynucleic acid comprises a plurality of modified internucleotide linkages, and at least 1, 2, 3, or 4 of the plurality of modified internucleotide linkages are stereochemically enriched phosphorothioate internucleotide linkages. In some instances, the stereochemically enriched phosphorothioate internucleotide linkages comprise both R- and S-isomers. In some instances, the at least one stereochemically enriched phosphorothioate is disposed between two consecutive nucleosides that are two of six 5′ or 3′-terminal nucleosides of the sense strand or the antisense strand.

In some instances, the polynucleic acid molecule comprises a hypoxanthine nucleobase-containing nucleoside substitution. In some instances, the hypoxanthine nucleobase-containing nucleoside substitution is an inosine substitution. In some instances, the inosine substitution is within a seed region of the antisense strand. In some instances, the inosine substitution is within 7 nucleotides from the 5′ end of the antisense strand. In some instances, the polynucleic acid molecule comprises an abasic substitution. In some instances, the abasic substitution is at the 5^{th} or 7^{th} nucleotide from the 5′ end.

In some instances, the cytotoxicity of the polynucleic acid molecule is decreased compared to unmodified polynucleic acid.

In some instances, the sense strand comprises a nucleic acid sequence that is at least 80%, at least 85%, at least 90%, at least 95% identical to a sequence selected from SEQ ID NOs: 13, 15, 17, 19, 21, and 128-230. In some instances, the sense strand comprises a nucleic acid sequence comprising at least 14, 15, 16, 17, 18, 19, or 20 consecutive nucleotides of a sequence selected from SEQ ID NOs: 13, 15, 17, 19, 21, and 128-230 with no more than 1, 2, 3, or 4 mismatches. In other instances, the antisense strand comprises a nucleic acid sequence that is at least 80%, at least 85%, at least 90%, at least 95% identical to a sequence selected from SEQ ID NOs: 14, 16, 18, 20, 22, and 334-436. In other instances, the antisense strand comprises a nucleic acid sequence comprising at least 14, 15, 16, 17, 18, 19, or 20 consecutive nucleotides of a sequence selected from SEQ ID NOs: 14, 16, 18, 20, 22, and 334-436 with no more than 1, 2, 3, or 4 mismatches. In some instances the sense strand comprises a sequence selected from a nucleic acid sequence of SEQ ID NOs: 13, 15, 17, 19, 21, and 128-230 and the antisense strand comprises a sequence selected from a nucleic acid sequence of SEQ ID NOs: 14, 16, 18, 20, 22, and 334-436. In some instances, the sense strand comprises a nucleic acid sequence that is at least 80%, at least 85%, at least 90%, at least 95% identical to a sequence selected from SEQ ID NOs: 13, 15, 17, 19, and 21. In some instances, the sense strand comprises a nucleic acid sequence comprising at least 14, 15, 16, 17, 18, 19, or 20 consecutive nucleotides of a sequence selected from SEQ ID NOs: 13, 15, 17, 19, and 21 with no more than 1, 2, 3, or 4 mismatches. In other instances, the antisense strand comprises a nucleic acid sequence that is at least 80%, at least 85%, at least 90%, at least 95% identical to a sequence selected from SEQ ID NOs: 14, 16, 18, 20, and 22. In other instances, the antisense strand comprises a nucleic acid sequence comprising at least 14, 15, 16, 17, 18, 19, or 20 consecutive nucleotides of a sequence selected from SEQ ID NOs: 14, 16, 18, 20, and 22 with no more than 1, 2, 3, or 4 mismatches. In some instances the sense strand comprises a sequence selected from a nucleic acid sequence of SEQ ID NOs: 13, 15, 17, 19, and 21 and the antisense strand comprises a sequence selected from a nucleic acid sequence of SEQ ID NOs: 14, 16, 18, 20, and 22.

In some instances, the polynucleic acid molecule is 19-25 base pairs in length. In some instances, the polynucleic acid molecule is 16-30 base pairs in length. In other instances, the polynucleic acid molecule is 21-23 base pairs in length. In other instances, the polynucleic acid molecule is 19-25 base pairs in length.

A polynucleic acid molecule for modulating expression of proprotein convertase subtilisin/kexin type 9 serine protease (PCSK9) gene, wherein polynucleic acid molecule comprises: (a) an antisense strand comprising the nucleotide sequence of usUfsacaaaagcaAfaAfcAfggucusasg (SEQ ID NO: 14) and a sense strand comprising the nucleotide sequence of asgsaccuGfuUfuUfgcuuuuguaa (SEQ ID NO:13); or (b) an antisense strand comprising the nucleotide sequence of usUfsucaaguuacAfaAfaGfcaaaascsa-(SEQ ID NO:16) and a sense strand comprising the nucleotide sequence of ususuugcUfuUfuGfuaacuugaaa (SEQ ID NO:15); wherein smaller case “n” stands for 2′-O-methyl modified nucleotide, upper case followed with an “f” (i.e., “Nf”) stands for 2′-fluoro modified nucleotide, and “s” stands for 3′-phosphorothioate.

In some aspects, disclosed herein are polynucleic acid molecule conjugates for modulating expression of PCSK9 gene, wherein the polynucleic acid molecule conjugate comprises a polynucleic acid molecules described herein and an asialoglycoprotein receptor targeting moiety. In some instances, the polynucleic acid molecule and the asialoglycoprotein receptor targeting moiety is coupled via a linker. In some instances, the linker comprises formula (IV) below,

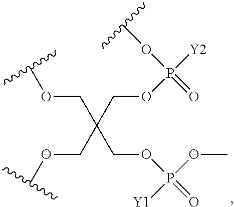

wherein at least one of Y1 and Y2 is a nucleotide in the polynucleic acid molecule. In some instances, the Y1 is the last nucleotide on the 3′-terminus of the sense strand of the polynucleic acid molecule. In other instances, the Y1 and Y2 are two consecutive nucleotides in the polynucleic acid molecule. In some instances, asialoglycoprotein receptor targeting moiety comprises N-Acetylgalactosamine (GalNAc). In some instances, the linker and the asialoglycoprotein receptor targeting moiety with the last nucleotide on the 3′-terminus of the sense strand of the polynucleic acid molecule are shown in Formula (V′):

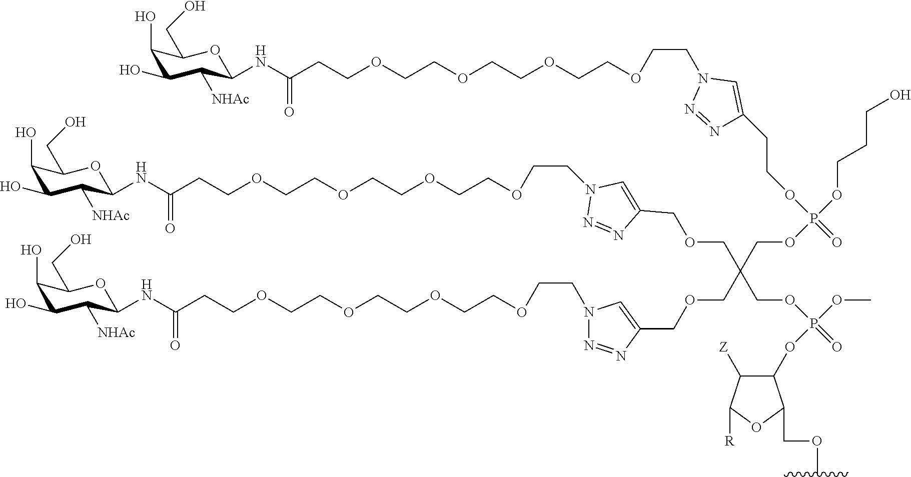

(V′), wherein Z in formula (V′) is —H, —OH, —O-Methyl, —F, or —O-methoxyethyl, and R in formula (V′) is adenine, uracil, guanine, cytosine, thymine, abasic, or others.

In some aspects, provided herein are pharmaceutical compositions comprising a polynucleic acid molecules described herein or a polynucleic acid molecule conjugates described herein, and a pharmaceutically acceptable excipient. In some instances, the pharmaceutical composition is formulated as a nanoparticle formulation. In some instances, the pharmaceutical composition is formulated for parenteral, oral, intranasal, buccal, rectal, transdermal, intravenous, subcutaneous, or intrathecal administration.

In some aspects, provided herein are methods of modulating expression of PCSK9 gene in a subject, comprising: administering to the subject the polynucleic acid molecules described herein, the polynucleic acid molecule conjugates described herein, or the pharmaceutical compositions described herein, thereby modulating the expression of PCSK9 gene in the subject.

In other aspects, provided herein are methods of modulating low-density lipoproteins (LDL) in a subject in need thereof, comprising: administering to the subject the polynucleic acid molecules described herein, the polynucleic acid molecule conjugates described herein, or the pharmaceutical compositions described herein, wherein the polynucleic acid molecules described herein, polynucleic acid molecule conjugates described herein, or pharmaceutical compositions described herein reduces the expression of PCSK9 gene in the subject. In some instances, the subject in need thereof suffers from hypercholesterolemia, familial hypercholesterolemia, or other high cholesterol-associated diseases.

In some aspects, provided herein are methods of modulating cholesterol in a subject in need thereof, comprising: administering to the subject the polynucleic acid molecules described herein, the polynucleic acid molecule conjugates described herein, or the pharmaceutical compositions described herein, wherein the polynucleic acid molecules described herein, the polynucleic acid molecule conjugates described herein, or the pharmaceutical compositions described herein reduces the expression of PCSK9 gene in the subject. In some instances, the subject in need thereof suffers from hypercholesterolemia, familial hypercholesterolemia, or other high cholesterol-associated diseases.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIGS. 1A-1J show effects of multiple siRNAs on PCSK9 level, LDL-c level, triglyceride (TG) levels, HDL-c levels, and total cholesterol (T-Chol) level in non-human primates.

### DETAILED DESCRIPTION OF THE DISCLOSURE

PCSK9 is the ninth member of the mammalian family of serine proteinases, a group of protein convertases that cleave inactive secretory precursors into bioactive proteins and peptides. The human PCSK9 gene is located on chromosome 1p32.3 and is translated into a ˜82-kDa zymogen in the endoplasmic reticulum (see Abifadel et al., 2003, Nat. Genet. 34, 154-156.; Piper et al., 2007, Structure 15, 545-552). PCSK9 is mainly secreted by hepatocytes into the blood stream and exists in the plasma.

PCSK9 has been shown to be involved in the degradation of LDL-cholesterol (LDL-C) and a subsequent condition hypercholesterolemia (see Abifadel et al., 2003, Nat. Genet. 34, 154-156). Hypercholesterolemia occurs as a consequence of a high-fat diet, inactivity, and combined with certain genetic risk factors. High levels of LDL are well-recognized risk factors. Exemplary genetic risk factors that are associated with hypercholesterolemia include mutations of genes encoding proteins that influence the level of LDL in the blood. For example, gain-of-function mutations in the gene encoding PCSK9 are associated with hypercholesterolemia. Upon PCSK9's binding to the LDLR, LDLR undergoes degradation, which consequently reduces the uptake of LDL-C from the bloodstream, thereby leading to hypercholesterolemia. Therefore, inhibition of PCSK9 is viewed as a potential therapeutic strategy to treat hypercholesterolemia, especially familial hypercholesterolemia, and other cholesterol-associated diseases.

Besides livers, PCSK9 is expressed in the small intestines, kidneys, and brains. Specifically, PCSK9 has been involved in neuroinflammation (see Apaijai et al., 2019, J. Am. Heart Assoc. 8:e010838). In addition, PCSK9 is associated with pathogenesis of Alzheimer's Disease. Brain autopsies reveal elevated PCSK9 mRNA and protein levels in the frontal cortices of late-onset AD patients compared to controls (see Picard et al., 2019, PLoS One 14:e0220254). Furthermore, PCSK9 participates in AUD, with an observation that PCSK9 levels in the CSF of patients with AUD were significantly higher compared to controls (see Chen et al., 2014, Lipids 49, 445-455). Furthermore, ischemic stroke is associated with several gain-of-function mutations in the PCSK9 gene that cause increased plasma LDL-C (see Rousselet et al., 2011, J. Lipid Res. 52, 1383-1391). Therefore, developing therapeutics targeting PCSK9 can be helpful for the above-mentioned neuronal disorders.

Described herein is a polynucleic acid molecule for modulating expression of PCSK9 gene, wherein the polynucleic acid molecule comprises a sense strand and an antisense strand, and wherein the polynucleic acid molecule comprises a nucleic acid sequence in Table 1, Table 2, and Table 3. Accordingly, provided herein are various target regions of human PCSK9 mRNA the polynucleic acid molecule described herein hybridizes to. In some embodiments, provided herein is the sequences of the polynucleic acid molecule described herein. In some embodiments, provided herein is the possible modifications of the polynucleic acid molecule described herein. In some embodiments, provided herein is the possible conjugates of the polynucleic acid molecule described herein.

Also described herein is a method of modulating expression of proprotein convertase subtilisin/kexin type 9 serine protease (PCSK9) gene in a subject. Described further herein is a method of modulating LDL and/or cholesterol in a subject in need thereof.

### Polynucleic Acid Molecules

### Target Regions of Polynucleic Acid Molecules

Described herein is a polynucleic acid molecule for modulating expression of PCSK9 gene. In some aspects, the polynucleic acid molecule is a single-stranded nucleic acid molecule that hybridizes to certain regions of mRNA. In some aspects, the polynucleic acid molecule is a double-stranded nucleic acid molecule. In some instances, the polynucleic acid molecule comprises a sense strand and an antisense strand, and wherein the antisense strand hybridizes to certain regions of PCSK9 mRNA.

In some aspects, the polynucleic acid molecule described herein hybridizes to certain regions of human PCSK9 mRNA. In some aspects, the polynucleic acid molecule described herein hybridizes to certain regions of non-human PCSK9 mRNA.

In some aspects, the polynucleic acid molecule described herein hybridizes to the 5′ UTR region of human PCSK9 mRNA. In some aspects, the polynucleic acid molecule described herein hybridizes to the coding region of human PCSK9 mRNA. In some aspects, the polynucleic acid molecule described herein hybridizes to the 3′ UTR region of human PCSK9 mRNA. In some specific aspects, the polynucleic acid molecule described herein hybridizes to a subset of 3′ UTR of human PCSK9 mRNA (NCBI Reference Sequence: NM-174936.3) with a range of transcription starting sites from 2342 to 2441, from 2442 to 2541, from 2542 to 2641, from 2642 to 2741, from 2742 to 2841, from 2842 to 2941, from 2942 to 3041, from 3042 to 3141, from 3142 to 3241, from 3242 to 3341, from 3342 to 3441, from 3442 to 3541, from 3542 to 3641, from 3642 to 3731.

In some aspects, the target region that the polynucleic acid molecule described herein hybridizes to is determined by an algorithm that predicts the maximal PCSK9 silencing effectiveness and lowest possible off-target effects. In some specific embodiment, the algorithm is disclosed in He et al., 2017, Scientific Reports, 7, 44836. In some specific embodiment, the algorithm is disclosed in Han et al., 2018, BMC Genomics 19, 669. In some specific embodiment, the algorithm is siRNArules, siRNA-Finder, siRNA Wizard™, siDirect, sima wizard, Dharmacon siRNA designing tool, White head siRNA designing tool, or Genscript siRNA software.

### Structure of Polynucleic Acid Molecules

### Single-Stranded Nucleic Acid Molecule

Described herein is a polynucleic acid molecule for modulating expression of PCSK9 gene, wherein the polynucleic acid molecule single-stranded nucleic acid molecule that is reverse complementary to the target region of PCSK9 mRNA as described above.

In some aspects, the polynucleic acid molecule described herein is not 100% complementary to the target region of PCSK9 mRNA. Accordingly, in some instances, the polynucleic acid molecule described herein is about 95% complementary to the target region of PCSK9 mRNA. In some specific aspects, the polynucleic acid molecule described herein is about 90% complementary to the target region of PCSK9 mRNA. In some specific aspects, the polynucleic acid molecule described herein is about 85% complementary to the target region of PCSK9 mRNA. In some specific aspects, the polynucleic acid molecule described herein is about 80% complementary to the target region of PCSK9 mRNA. In some specific aspects, the polynucleic acid molecule described herein is about 75% complementary to the target region of PCSK9 mRNA. In some specific aspects, the polynucleic acid molecule described herein is about 70% complementary to the target region of PCSK9 mRNA.

In some aspects, the polynucleic acid molecule described herein comprises a nucleic acid sequence in Table 1, Table 2, and Table 3. In other aspects, the polynucleic acid molecule described herein comprises a nucleic acid sequence that is at least 80%, at least 85%, at least 90%, at least 95% identical to a sequence in Table 1, Table 2, and Table 3. In some instances, the polynucleic acid molecule described herein comprises at least 80%, at least 85%, at least 90%, at least 95% complementary to a sequence selected from SEQ ID NOs:1, 3, 5, 7, 9, 25-127, and 437-539. In some instances, the polynucleic acid molecule described herein comprises at least 80%, at least 85%, at least 90%, at least 95% complementary to a sequence selected from SEQ ID NOs: 1, 3, 5, 7, and 9.

In yet other aspects, the polynucleic acid molecule described herein comprises a nucleic acid sequence that is 15 consecutive nucleotides out of the sequences in Table 1, Table 2, and Table 3 with no more than 2, 3, or 4 mismatches. In specific aspects, the polynucleic acid molecule described herein comprises a nucleic acid sequence that is 15 consecutive nucleotides that are complementary to a nucleic acid sequence of SEQ ID NOs: 1, 3, 5, 7, 9, 25-127, and 437-539 with no more than 2, 3, or 4 mismatches. In yet still other aspects, the polynucleic acid molecule described herein comprises a nucleic acid sequence that is 16 consecutive nucleotides out of the sequences in Table 1, Table 2, and Table 3 with no more than 2, 3, or 4 mismatches. In specific aspects, the polynucleic acid molecule described herein comprises a nucleic acid sequence that is 16 consecutive nucleotides that are complementary to a nucleic acid sequence of SEQ ID NOs: 1, 3, 5, 7, 9, 25-127, and 437-539 with no more than 2, 3, or 4 mismatches. In yet still other aspects, the polynucleic acid molecule described herein comprises a nucleic acid sequence that is 17 consecutive nucleotides out of the sequences in Table 1, Table 2, and Table 3 with no more than 2, 3, or 4 mismatches. In specific aspects, the polynucleic acid molecule described herein comprises a nucleic acid sequence that is 17 consecutive nucleotides that are complementary to a nucleic acid sequence of SEQ ID NOs: 1, 3, 5, 7, 9, 25-127, and 437-539 with no more than 2, 3, or 4 mismatches. In yet other aspects, the polynucleic acid molecule described herein comprises a nucleic acid sequence that is 18 consecutive nucleotides out of the sequences in Table 1, Table 2, and Table 3 with no more than 2, 3, or 4 mismatches. In specific aspects, the polynucleic acid molecule described herein comprises a nucleic acid sequence that is 18 consecutive nucleotides that are complementary to a nucleic acid sequence of SEQ ID NOs: 1, 3, 5, 7, 9, 25-127, and 437-539 with no more than 2, 3, or 4 mismatches. In yet other aspects, the polynucleic acid molecule described herein comprises a nucleic acid sequence that is 19 consecutive nucleotides out of the sequences in Table 1, Table 2, and Table 3 with no more than 2, 3, or 4 mismatches. In specific aspects, the polynucleic acid molecule described herein comprises a nucleic acid sequence that is 19 consecutive nucleotides that are complementary to a nucleic acid sequence of SEQ ID NOs: 1, 3, 5, 7, 9, 25-127, and 437-539 with no more than 2, 3, or 4 mismatches. In yet other aspects, the polynucleic acid molecule described herein comprises a nucleic acid sequence that is 20 consecutive nucleotides out of the sequences in Table 1, Table 2, and Table 3 with no more than 2, 3, or 4 mismatches. In specific aspects, the polynucleic acid molecule described herein comprises a nucleic acid sequence that is 20 consecutive nucleotides that are complementary to a nucleic acid sequence of SEQ ID NOs: 1, 3, 5, 7, 9, 25-127, and 437-539 with no more than 2, 3, or 4 mismatches. In yet other aspects, the polynucleic acid molecule described herein comprises a nucleic acid sequence that is 21 consecutive nucleotides out of the sequences in Table 1, Table 2, and Table 3 with no more than 2, 3, or 4 mismatches. In specific aspects, the polynucleic acid molecule described herein comprises a nucleic acid sequence that is 21 consecutive nucleotides that are complementary to a nucleic acid sequence of SEQ ID NOs: 1, 3, 5, 7, 9, 25-127, and 437-539 with no more than 2, 3, or 4 mismatches. In yet other aspects, the polynucleic acid molecule described herein comprises a nucleic acid sequence that is 22 consecutive nucleotides out of the sequences in Table 1, Table 2, and Table 3 with no more than 2, 3, or 4 mismatches. In specific aspects, the sense strand described herein comprises a nucleic acid sequence that is 22 consecutive nucleotides that are complementary to a nucleic acid sequence of SEQ ID NOs: 1, 3, 5, 7, 9, 25-127, and 437-539 with no more than 2, 3, or 4 mismatches.

In specific aspects, the polynucleic acid molecule described herein comprises a nucleic acid sequence that is 15 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 16 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 17 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 18 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 19 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 20 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 21 consecutive nucleotides that are complementary to a nucleic acid sequence of SEQ ID NOs: 1, 3, 5, 7, and 9 with no more than 1, 2, or 3 mismatches. In specific aspects, the polynucleic acid molecule described herein comprises a nucleic acid sequence that is 22 consecutive nucleotides that are complementary to a nucleic acid sequence of SEQ ID NOs: 1, 3, 5, 7, and 9 with no more than 1, 2, or 3 mismatches.

In some aspects, the polynucleic acid molecule described herein comprises about 15-30, 16-30, 17-30, 18-30, 18-27, 18-25, 18-23, 19-23, 20-23, or 21-23 nucleotides in length. In some aspects, the polynucleic acid molecule described herein comprises about 15, 16, 17, 18, 19, 20 nucleotides long. In some aspects, the polynucleic acid molecule described herein comprises about 21, 22, 23, 24, 25 nucleotides long. In some aspects, the polynucleic acid molecule described herein comprises about 26, 27, 28, 29, 30 nucleotides long. In some specific aspects, the polynucleic acid molecule described herein comprises 19 nucleotides long. In some specific aspects, the polynucleic acid molecule described herein comprises 21 nucleotides long. In some specific aspects, the polynucleic acid molecule described herein comprises 23 nucleotides long.

### Double-Stranded Nucleic Acid Molecule

Described herein is a polynucleic acid molecule for modulating expression of PCSK9 gene, wherein the polynucleic acid molecule is a double-stranded molecule that comprises a sense strand and an antisense strand, and wherein the antisense strand is reverse complementary to the target region of PCSK9 mRNA as described above.

In some aspects, the antisense strand described herein is 100% complementary to the target region of PCSK9 mRNA. In some aspects, the antisense strand described herein is not 100% complementary to the target region of PCSK9 mRNA. Accordingly, in some instances, the antisense strand described herein is about 95% complementary to the target region of PCSK9 mRNA. In some specific aspects, the antisense strand described herein is about 90% complementary to the target region of PCSK9 mRNA. In some specific aspects, the antisense strand described herein is about 85% complementary to the target region of PCSK9 mRNA. In some specific aspects, the antisense strand described herein is about 80% complementary to the target region of PCSK9 mRNA. In some specific aspects, the antisense strand described herein is about 75% complementary to the target region of PCSK9 mRNA. In some specific aspects, the antisense strand described herein is about 70% complementary to the target region of PCSK9 mRNA.

In some aspects, the polynucleic acid molecule described herein comprises a nucleic acid sequence in Table 1, Table 2, and Table 3. In other aspects, the polynucleic acid molecule described herein comprises a nucleic acid sequence that is at least 80%, at least 85%, at least 90%, at least 95% identical to a sequence in Table 1, Table 2, and Table 3. In some instances, the sense strand described herein comprises at least 80%, at least 85%, at least 90%, at least 95% identical to a sequence selected from SEQ ID NOs:1, 3, 5, 7, 9, 25-127, and 437-539. In some instances, the antisense strand described herein comprises at least 80%, at least 85%, at least 90%, at least 95% identical to a sequence selected from SEQ ID NOs:2, 4, 6, 8, 10, and 231-333. In some instances, the sense strand described herein comprises at least 80%, at least 85%, at least 90%, at least 95% identical to a sequence selected from SEQ ID NOs: 1, 3, 5, 7, and 9. In some instances, the antisense strand described herein comprises at least 80%, at least 85%, at least 90%, at least 95% identical to a sequence selected from SEQ ID NOs: 2, 4, 6, 8, and 10.

In yet other aspects, the polynucleic acid molecule described herein comprises a nucleic acid sequence that is 14 consecutive nucleotides out of the sequences in Table 1, Table 2, and Table 3 with no more than 1, 2, 3, or 4 mismatches. In specific aspects, the sense strand described herein comprises a nucleic acid sequence that is 14 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 14 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 15 consecutive nucleotides out of the sequences in Table 1, Table 2, and Table 3 with no more than 1, 2, 3, or 4 mismatches. In specific aspects, the sense strand described herein comprises a nucleic acid sequence that is 15 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 15 consecutive nucleotides of SEQ ID NOs: 2, 4, 6, 8, 10, and 231-333 with no more than 1, 2, 3, or 4 mismatches. In yet still other aspects, the polynucleic acid molecule described herein comprises a nucleic acid sequence that is 16 consecutive nucleotides out of the sequences in Table 1, Table 2, and Table 3 with no more than 1, 2, 3, or 4 mismatches. In specific aspects, the sense strand described herein comprises a nucleic acid sequence that is 16 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 16 consecutive nucleotides of SEQ ID NOs: 2, 4, 6, 8, 10, and 231-333 with no more than 1, 2, 3, or 4 mismatches. In yet still other aspects, the polynucleic acid molecule described herein comprises a nucleic acid sequence that is 17 consecutive nucleotides out of the sequences in Table 1, Table 2, and Table 3 with no more than 1, 2, 3, or 4 mismatches. In specific aspects, the sense strand described herein comprises a nucleic acid sequence that is 17 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 17 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 18 consecutive nucleotides out of the sequences in Table 1, Table 2, and Table 3 with no more than 1, 2, 3, or 4 mismatches. In specific aspects, the sense strand described herein comprises a nucleic acid sequence that is 18 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 18 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 19 consecutive nucleotides out of the sequences in Table 1, Table 2, and Table 3 with no more than 1, 2, 3, or 4 mismatches. In specific aspects, the sense strand described herein comprises a nucleic acid sequence that is 19 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 19 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 20 consecutive nucleotides out of the sequences in Table 1, Table 2, and Table 3 with no more than 1, 2, 3, or 4 mismatches. In specific aspects, the sense strand described herein comprises a nucleic acid sequence that is 20 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 20 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 21 consecutive nucleotides out of the sequences in Table 1, Table 2, and Table 3 with no more than 1, 2, 3, or 4 mismatches. In specific aspects, the sense strand described herein comprises a nucleic acid sequence that is 21 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 21 consecutive nucleotides of SEQ ID NOs: 2, 4, 6, 8, 10, and 231-333 with no more than 1, 2, 3, or 4 mismatches. In yet other aspects, the polynucleic acid molecule described herein comprises a nucleic acid sequence that is 22 consecutive nucleotides out of the sequences in Table 1, Table 2, and Table 3 with no more than 1, 2, 3, or 4 mismatches. In specific aspects, the sense strand described herein comprises a nucleic acid sequence that is 22 consecutive nucleotides of SEQ ID NOs: 1, 3, 5, 7, 9, 25-127, and 437-539 with no more than 1, 2, 3, or 4 mismatches. In specific aspects, the antisense strand described herein comprises a nucleic acid sequence that is 22 consecutive nucleotides of SEQ ID NOs: 2, 4, 6, 8, 10, and 231-333 with no more than 1, 2, 3, or 4 mismatches.

In specific aspects, the sense strand described herein comprises a nucleic acid sequence that is 14 consecutive nucleotides of SEQ ID NOs: 1, 3, 5, 7, and 9 with no more than 1, 2, or 3 mismatches. In specific aspects, the antisense strand described herein comprises a nucleic acid sequence that is 14 consecutive nucleotides of SEQ ID NOs: 2, 4, 6, 8, and 10 with no more than 1, 2, or 3 mismatches. In specific aspects, the sense strand described herein comprises a nucleic acid sequence that is 15 consecutive nucleotides of SEQ ID NOs: 1, 3, 5, 7, and 9 with no more than 1, 2, or 3 mismatches. In specific aspects, the antisense strand described herein comprises a nucleic acid sequence that is 15 consecutive nucleotides of SEQ ID NOs: 2, 4, 6, 8, and 10 with no more than 1, 2, or 3 mismatches. In specific aspects, the sense strand described herein comprises a nucleic acid sequence that is 16 consecutive nucleotides of SEQ ID NOs: 1, 3, 5, 7, and 9 with no more than 1, 2, or 3 mismatches. In specific aspects, the antisense strand described herein comprises a nucleic acid sequence that is 16 consecutive nucleotides of SEQ ID NOs: 2, 4, 6, 8, and 10 with no more than 1, 2, or 3 mismatches. In specific aspects, the sense strand described herein comprises a nucleic acid sequence that is 17 consecutive nucleotides of SEQ ID NOs: 1, 3, 5, 7, and 9 with no more than 1, 2, or 3 mismatches. In specific aspects, the antisense strand described herein comprises a nucleic acid sequence that is 17 consecutive nucleotides of SEQ ID NOs: 2, 4, 6, 8, and 10 with no more than 1, 2, or 3 mismatches. In specific aspects, the sense strand described herein comprises a nucleic acid sequence that is 18 consecutive nucleotides of SEQ ID NOs: 1, 3, 5, 7, and 9 with no more than 1, 2, or 3 mismatches. In specific aspects, the antisense strand described herein comprises a nucleic acid sequence that is 18 consecutive nucleotides of SEQ ID NOs: 2, 4, 6, 8, and 10 with no more than 1, 2, or 3 mismatches. In specific aspects, the sense strand described herein comprises a nucleic acid sequence that is 19 consecutive nucleotides of SEQ ID NOs: 1, 3, 5, 7, and 9 with no more than 1, 2, or 3 mismatches. In specific aspects, the antisense strand described herein comprises a nucleic acid sequence that is 19 consecutive nucleotides of SEQ ID NOs: 2, 4, 6, 8, and 10 with no more than 1, 2, or 3 mismatches. In specific aspects, the sense strand described herein comprises a nucleic acid sequence that is 20 consecutive nucleotides of SEQ ID NOs: 1, 3, 5, 7, and 9 with no more than 1, 2, or 3 mismatches. In specific aspects, the antisense strand described herein comprises a nucleic acid sequence that is 20 consecutive nucleotides of SEQ ID NOs: 2, 4, 6, 8, and 10 with no more than 1, 2, or 3 mismatches. In specific aspects, the sense strand described herein comprises a nucleic acid sequence that is 21 consecutive nucleotides of SEQ ID NOs: 1, 3, 5, 7, and 9 with no more than 1, 2, or 3 mismatches. In specific aspects, the antisense strand described herein comprises a nucleic acid sequence that is 21 consecutive nucleotides of SEQ ID NOs: 2, 4, 6, 8, and 10 with no more than 1, 2, or 3 mismatches. In specific aspects, the sense strand described herein comprises a nucleic acid sequence that is 22 consecutive nucleotides of SEQ ID NOs: 1, 3, 5, 7, and 9 with no more than 1, 2, or 3 mismatches. In specific aspects, the antisense strand described herein comprises a nucleic acid sequence that is 22 consecutive nucleotides of SEQ ID NOs: 2, 4, 6, 8, and 10 with no more than 1, 2, or 3 mismatches.

In some aspects, the polynucleic acid molecule described herein comprises a sense and an antisense strand of about 15-30, 16-30, 17-30, 18-30, 18-27, 18-25, 18-23, 19-23, 20-23, or 21-23 nucleotides in length. In some aspects, the polynucleic acid molecule described herein comprises a sense and an antisense strand of about 15, 16, 17, 18, 19, 20 nucleotides long. In some aspects, the polynucleic acid molecule described herein comprises a sense and an antisense strand of about 21, 22, 23, 24, 25 nucleotides long. In some aspects, the polynucleic acid molecule described herein comprises a sense and an antisense strand of about 26, 27, 28, 29, 30 nucleotides long. In some specific aspects, the polynucleic acid molecule described herein comprises a sense strand of 19 nucleotides long, and an antisense strand of about 21 nucleotides long. In some specific aspects, the polynucleic acid molecule described herein comprises a sense strand of 21 nucleotides long, and an antisense strand of about 23 nucleotides long.

In some aspects, the sense strand and the antisense strand described herein are reverse complementary to each other and form a duplex with a 3′ overhang on the antisense strand. In some aspects, the sense strand and the antisense strand described herein are reverse complementary to each other and form a duplex with a 5′ overhang on the antisense strand. In some aspects, the sense strand and the antisense strand described herein are reverse complementary to each other and form a duplex with a 3′ overhang on the sense strand. In some aspects, the sense strand and the antisense strand described herein are reverse complementary to each other and form a duplex with a 5′ overhang on the sense strand.

### Modifications of Polynucleic Acid Molecules

In some aspects, described herein is the polynucleic acid molecule described herein with modifications. In some aspects, the modifications described herein occurs one or more different structures of the polynucleotide acid molecule described herein (e.g., modifications on sugar ring(s), backbone(s), base(s)). In some aspects, the modifications described herein comprise substitutions of one or more nucleotide in the polynucleic acid molecule described herein. In some aspects, different percentages of the polynucleic acid molecule described herein comprise the modifications described herein. In some aspects, different positions of the polynucleic acid molecule described herein comprise the modifications described herein. WO/2018/035380 is herein incorporated by reference in its entirety.

### Types of Modifications

In some aspects, the polynucleotide acid molecule described herein comprises one or more sugar-modified nucleotide. In some aspects, the sugar-modified nucleotide is a 2′-fluoro modified nucleotide. In some instances, the sugar-modified nucleotide includes a modification at a 2′ hydroxyl group of the ribose moiety. In some instances, the sugar-modified nucleotide includes modification with an H, OR, R, halo, SH, SR, NH2, NHR, NR2, or CN, wherein R is an alkyl moiety. In some aspects, the sugar-modified nucleotide is a 2′-O-methyl modified nucleotide or 2′-alkoxy modified nucleotide (e.g., 2′-methoxy modified nucleotide). In some instances, 2′ hydroxyl group modification includes 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some instances, the alkyl moiety comprises a hetero substitution. In some instances, the carbon of the heterocyclic group is substituted by a nitrogen, oxygen or sulfur. In some aspects, the sugar-modified nucleotide is a 2′-amino modified nucleotide. In some aspects, the sugar-modified nucleotide is a 2′-azido modified nucleotide. In some aspects, the sugar-modified nucleotide is a 2′-deoxy modified nucleotide. In some aspects, the sugar-modified nucleotide is a 2′-O-methoxythyl (2′-MOE). In some aspects, the sugar-modified nucleotide is a locked nucleic acid (LNA). In some aspects, the sugar-modified nucleotide is an ethylene-bridged nucleic acid (ENA). In some aspects, the sugar-modified nucleotide is a (S)-constrained ethyl (cEt). In some aspects, the sugar-modified nucleotide is a tricyclo-DNA (tcDNA). In some aspects, the sugar-modified nucleotide is a 2′-NH2 nucleic acid.

In some aspects, the polynucleotide acid molecule described herein comprises one or more sugarphosphate-modified nucleotide. In some aspects, the modified sugarphosphate is phosphorodiamidate morpholino (PMO). In some aspects, the modified sugarphosphate is phosphoramidate. In some instances, the heterocyclic substitution includes imidazole, and pyrrolidino. In some aspects, the modified sugarphosphate is thiophosphoramidate. In some aspects, the modified sugarphosphate is peptide nucleic acid (PNA).

In some aspects, the polynucleotide acid molecule described herein comprises one or more backbone-modified nucleotide. In some specific aspects, the modified backbone is a methylphosphonate. In some specific aspects, the modified backbone is phosphorothioate. In some specific aspects, the modified backbone is a guanidinopropyl phosphoramidate. In some specific aspects, the modified backbone is a mesyl-phosphoramidate (MsPA) linkages. In some instances, the modified backbone comprises one or more of phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphoramidates.

In some aspects, the modified nucleotide comprises a modified guanine (e.g., inosine) or one or more of any types of unnatural nucleic acids.

In some specific aspects, the modified backbone is phosphorothioate, and the phosphorothioate is a stereochemically enriched phosphorothioate. In certain aspects, the strand contains at least one stereochemically enriched phosphorothioate. In some aspects, the strand comprises at least 1, 2, 3 stereochemically enriched phosphorothioates. In some aspects, the strand comprises only 1, 2, 3, or 4 stereochemically enriched phosphorothioates. In further aspects, at least one (e.g., one or two) stereochemically enriched phosphorothioate is disposed between two consecutive nucleosides that are two of six 5′-terminal nucleosides of the strand. In yet further aspects, at least one (e.g., one or two) stereochemically enriched phosphorothioate is disposed between two consecutive nucleosides that are two of six 3′-terminal nucleosides of the strand. In still further aspects, one stereochemically enriched phosphorothioate is covalently bonded to the first nucleoside and the second nucleoside from the 5′-end within the strand. In some aspects, one stereochemically enriched phosphorothioate is covalently bonded to the twenty first nucleoside and the twenty second nucleoside from the 5′-end within the strand. In certain aspects, one stereochemically enriched phosphorothioate is covalently bonded to the twenty second nucleoside and the twenty third nucleoside from the 5′-end within the strand. In particular aspects, the stereochemically enriched phosphorothioate has RP stereochemical identity. In certain aspects, the stereochemically enriched phosphorothioate has SP stereochemical identity.

In some aspects, the polynucleotide molecules described herein comprises one or more (e.g., from 1 to 20, from 1 to 10, or from 1 to 5) stereochemically enriched (e.g., internucleoside) phosphorothioates (e.g., having diastereomeric excess of at least 10%, at least 50%, at least 60%, at least 70%, at least 80%, or at least 90%, e.g., up to about 99%, for the P-stereogenic center). The polynucleotide molecules described herein comprises one or more (e.g., from 1 to 20, from 1 to 10, or from 1 to 5; e.g., internucleoside) phosphorodithioates. The phosphorodithioates may be non-P-stereogenic in the polynucleotide molecules described herein. Phosphorothioates and phosphorodithioates may enhance the stability of the polynucleotide molecules described herein to exonuclease activity of serum. Non-P-stereogenic phosphorodithioates may simplify the synthesis of the polynucleotide molecule described herein by reducing the number of possible diastereomers. Typically, the phosphorothioate or phosphorodithioate may connect two contiguous nucleosides within the six 3′-terminal nucleosides and the six 5′-terminal nucleosides of the polynucleotide molecules described herein. In some aspects, the stereochemically enriched phosphorothioate (e.g., RP-enriched phosphorothioate) may be covalently bonded to the first nucleoside (e.g., the 3′-carbon atom of the first nucleoside) and the second nucleoside (e.g., the 5′-carbon atom of the second nucleoside) from the 5′-end of the antisense strand. Additionally or alternatively, the stereochemically enriched phosphorothioate (e.g., SP-enriched phosphorothioate) may be covalently bonded to the 21^{st} nucleoside (e.g., the 3′-carbon atom of the 21^{st} nucleoside) from the 5′-end and the 22^{nd} nucleoside (e.g., the 5′-carbon atom of the 22^{nd} nucleoside) of the antisense strand. Further, additionally or alternatively, the stereochemically enriched phosphorothioate (e.g., SP-enriched phosphorothioate) may be covalently bonded to the 22^{nd} nucleoside (e.g., the 3′-carbon atom of the 22^{nd} nucleoside) and the 23^{rd} nucleoside (e.g., the 5′-carbon atom of the 23^{rd} nucleoside) from the 5′-end of the antisense strand. Combinations of a 5′ RP-enriched phosphorothioate (e.g., RP-enriched phosphorothioate covalently bonded to the first nucleoside (e.g., the 3′-carbon atom of the first nucleoside) and the second nucleoside (e.g., the 5′-carbon atom of the second nucleoside) from the 5′-end and a 3′ SP-enriched phosphorothioate (e.g., SP-enriched phosphorothioate covalently bonded to the 21^{st} nucleoside (e.g., the 3′-carbon atom of the 21^{st} nucleoside) and the 22^{nd} nucleoside (e.g., the 5′-carbon atom of the 22^{nd} nucleoside) from the 5′-end in an antisense strand can produce superior efficacy and/or duration of action, e.g., as measured by the reduction in the activity of the target relative to a reference guide strand that lacks the combination of a 5′ RP-enriched phosphorothioate and a 3′ SP-enriched phosphorothioate.

In some aspects, the polynucleotide molecule described herein comprises one or more purine modification. In some specific aspects, the purine modification described herein is 2,6-diaminopurine. In some specific aspects, the purine modification described herein is 3-deaza-adenine. In some specific aspects, the purine modification described herein is 7-deaza-guanine. In some specific aspects, the purine modification described herein is 8-azido-adenine.

In some aspects, the polynucleotide molecule described herein comprises one or more pyrimidine modification. In some specific aspects, the pyrimidine modification described herein is 2-thio-thymidine. In some specific aspects, the pyrimidine modification described herein is 5-carboxamide-uracil. In some specific aspects, the pyrimidine modification described herein is 5-methyl-cytosine. In some specific aspects, the pyrimidine modification described herein is 5-ethynyl uracil.

In some embodiment, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the polynucleotide molecule disclosed herein may include one or more (e.g., one or two) abasic substitutions. In specific aspects, abasic substitution is at the 5^{th} nucleotide from the 5′ end of the antisense strand described herein. In specific aspects, abasic substitution is at the 7^{th} nucleotide from the 5′ end of the antisense strand described herein.

When the polynucleotide molecule disclosed herein includes two or more of the abasic substitutions, their structures may be same or different. In certain aspects, a sense strand contains one abasic substitution (e.g., an antisense strand may be free of abasic substitutions). In other aspects, an antisense strand contains one abasic substitution (e.g., a sense strand may be free of abasic substitutions). In yet other aspects, an antisense strand contains one abasic substitution, and a sense strand contains one abasic substitution. In further aspects, a sense strand includes an abasic substitution between a nucleoside number (x) and a nucleoside number (x+1), where x is an integer from 2 to 7. In yet further aspects, an antisense strand includes an abasic substitution between a nucleoside number (x) and a nucleoside number (x+1), where x is an integer from 2 to 7.

The abasic substitution may be of formula (III):

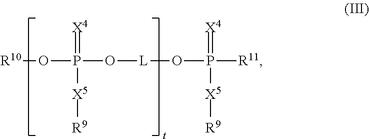

where
L is a sugar analogue, or is substituted with a heteroacyl from A, U, C, G, or is any other substituted nucleic acid (e.g., locked or unlocked nucleic acid, glycol nucleic acid, etc.;
each X⁴ is independently O or S;
each X⁵ is independently O, S, NH, or a bond;
each R⁹ is independently H, optionally substituted C₁₋₆ alkyl, optionally substituted C₂₋₆ alkenyl, optionally substituted C₂₋₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, -LinkA(-T)ₚ, or a conjugation moiety;
each LinkA is independently a multivalent linker (e.g., including —C(O)—N(H)—);
each T is independently an auxiliary moiety;
R¹⁰ is a bond to a 3′-carbon atom of a nucleoside (x) in the strand;
R¹¹ is a bond to a 5′-oxygen atom of a nucleoside (x+1) in the strand;
p is an integer from 1 to 6; and
t is an integer from 1 to 6.

In some aspects, the abasic substitution described herein is attached to the antisense strand of the polynucleic acid molecule described herein. In particular aspects, an abasic substitution (e.g., an internucleotide, abasic spacer of formula (III) in which t is 1) may be included in the antisense strand described herein (e.g., within the seed region of the guide strand). In some aspects, an abasic substitution (e.g., an internucleotide, abasic spacer of formula (III) in which t is 1) may be bonded to the 3′ carbon atom of the second, third, fourth, or fifth nucleoside from the 5′-end of the antisense strand described herein. In certain aspects, an abasic substitution (e.g., an internucleotide, abasic spacer of formula (III) in which t is 1) may be bonded to the 3′ carbon atom of the thirteenth, fourteenth, fifteenth, or sixteenth nucleoside from the 5′-end of the antisense strand described herein. In some aspects, an abasic substitution fourth, fifth, sixth, seventh, eighth, and/or ninth nucleoside from the 5′-end of the antisense strand described herein.

The polynucleotide molecule described herein may contain a strand including a seed region including a hypoxanthine nucleobase-containing nucleoside (e.g., inosine).

In certain aspects, the hypoxanthine nucleobase-containing nucleoside is a second nucleoside from the 5′-end in the strand. In further aspects, the hypoxanthine nucleobase-containing nucleoside is a third nucleoside from the 5′-end in the strand. In yet further aspects, the hypoxanthine nucleobase-containing nucleoside is a fourth nucleoside from the 5′-end in the strand. In still further aspects, the hypoxanthine nucleobase-containing nucleoside is a fifth nucleoside from the 5′-end in the strand. In particular aspects, the hypoxanthine nucleobase-containing nucleoside is a sixth nucleoside in the strand. In particular aspects, the hypoxanthine nucleobase-containing nucleoside is a seventh nucleoside in the strand.

### The Amount and Location of Modifications

In some aspects, the polynucleotide molecule described herein comprises one or more type of modifications as described above. Accordingly, in some aspects, about 10% of the nucleotides from the polynucleotide molecule described herein are modified with one or more type of modifications as described above. In other aspects, about 20% of the nucleotides from the polynucleotide molecule described herein are modified with one or more type of modifications as described above. In other aspects, about 30% of the nucleotides from the polynucleotide molecule described herein are modified with one or more type of modifications as described above. In other aspects, about 40% of the nucleotides from the polynucleotide molecule described herein are modified with one or more type of modifications as described above. In other aspects, about 50% of the nucleotides from the polynucleotide molecule described herein are modified with one or more type of modifications as described above. In other aspects, about 60% of the nucleotides from the polynucleotide molecule described herein are modified with one or more type of modifications as described above. In other aspects, about 70% of the nucleotides from the polynucleotide molecule described herein are modified with one or more type of modifications as described above. In other aspects, about 80% of the nucleotides from the polynucleotide molecule described herein are modified with one or more type of modifications as described above. In other aspects, about 90% of the nucleotides from the polynucleotide molecule described herein are modified with one or more type of modifications as described above. In other aspects, 100% of the nucleotides from the polynucleotide molecule described herein are modified with one or more type of modifications as described above.

In some aspects, the one or more types of modifications described herein occurs at different positions within the polynucleotide molecule described herein. In specific aspects, the one or more types of modifications described herein occurs in the seed region within the polynucleotide molecule described herein. In specific aspects, the one or more types of modifications described herein occurs at 3′ terminal of the polynucleotide molecule described herein. In specific aspects, the one or more types of modifications described herein occurs at 5′ terminal of the polynucleotide molecule described herein. In specific aspects, the one or more types of modifications described herein occurs dispersedly within the polynucleotide molecule described herein. In specific aspects, the one or more types of modifications described herein occurs in clusters within the polynucleotide molecule described herein.

### Specific Modification Patterns

In some aspects, described herein is a specific modification pattern for the polynucleic acid molecule which is a double-stranded nucleic acid molecule comprising a sense stand and an antisense strand, wherein the sense strand comprises about twelve 2′-fluoro modified nucleotides and about nine 2′-O-methyl modified nucleotides, and wherein the antisense strand comprises about nine 2′-fluoro modified nucleotides and about fourteen 2′-O-methyl modified nucleotides.

In some aspects, described herein is a specific modification pattern, wherein the sense strand is fully modified and comprises twelve 2′-fluoro modified nucleotides, nine 2′-O-methyl modified nucleotides, and wherein the antisense strand is fully modified and comprises nine 2′-fluoro modified nucleotides and fourteen 2′-O-methyl modified nucleotides.

In some aspects, described herein is a specific modification pattern, wherein the sense strand comprises ‘5-NfnNfnNfnNfnNfNfNfnNfnNfnNfnNfnNf-3’, wherein the antisense strand comprises ‘5-nNfnNfnNfnNfnNfnnnNfnNfnNfnNfnnn-3’, wherein “Nf” stands for a 2′-fluoro modified nucleotide, and wherein “n” stands for a 2′-O-methyl modified nucleotide.

In some aspects, described herein is a specific modification pattern, wherein the sense strand comprises ‘5-NfnNfnNfnNfnNfNfNfnNfnNfnNfnNfnNf-3’, wherein the antisense strand comprises ‘5-nNfnNfnNfnNfnNfnnnNfnNfnNfnNfnnn-3’, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises ‘5-NfnNfnNfnNfnNfNfNfnNfnNfnNfnNfnNf-3’, wherein the antisense strand comprises ‘5-nNfnNfnNfnNfnNfnnnNfnNfnNfnNfnnn-3’, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type I in Table 4.

TABLE 4
Various Modification Patterns

| Pattern Name | Pattern |
|---|---|
| Type I for sense strand | ‘5- NfsnsNfnNfnNfnNfNfNfnNfnNfnNfnNfnNf-3’ |
| Type I for antisense strand | ‘5-nsNfsnNfnNfnNfnNfnnnNfnNfnNfnNfnsnsn-3’ |
| Type II for sense strand | ‘5-nsnsnnnnNfnNfNfNfnnnnnnnnnn -3’ |
| Type II for antisense strand | ‘5-nsNfsnnnNfnNfNfnnnnNfnNfnnnnnsnsn -3’ |
| Type III for sense strand | ‘5-nsnsnnnnnnNfnNfnnnnnnnnnn -3’ |
| Type III for antisense strand | ‘5-nsNfsnnnnnnnnnNfnNfnnnnnnnsnsn -3’ |
| Type IV for sense strand | ‘5-nsnsnnnnNfnNfnNfnnnnnnnnnn -3’ |
| Type IV for antisense strand | ‘5-nsNfsnnnnnnnnnNfnNfnNfnnnnnsnsn -3’ |

Note:
“Nf” stands for a 2′-fluoro modified nucleotide, “n” stands for a 2′-O-methyl modified nucleotide, “s” stands for a 3′-phosphorothioate.

In some aspects, the polynucleotide molecule provided herein comprises a sense strand from one of SEQ ID NOs: 1, 3, 5, 7, 9, 25-127, and 437-539 and an antisense strand comprises one of SEQ ID NOs: 2, 4, 6, 8, 10, and 231-333. In other aspects, the polynucleotide molecule provided herein comprises a sense strand from one of SEQ ID NOs: 1, 3, 5, 7, 9, 25-127, and 437-539, an antisense strand comprises one of SEQ ID NOs: 2, 4, 6, 8, 10, and 231-333, and wherein the sense and/or antisense strand is modified in Type I modification pattern specified in Table 4. In some aspects, the polynucleotide molecule provided herein comprises a sense strand from one of SEQ ID NOs: 1, 3, 5, 7, and 9 and an antisense strand comprises one of SEQ ID NOs: 2, 4, 6, 8, and 10. In other aspects, the polynucleotide molecule provided herein comprises a sense strand from one of SEQ ID NOs: 1, 3, 5, 7, and 9, an antisense strand comprises one of SEQ ID NOs: 2, 4, 6, 8, and 10, and wherein the sense and/or antisense strand is modified in Type I modification pattern specified in Table 4.

In some aspects, described herein is a specific modification pattern, wherein the sense strand comprises about four 2′-fluoro modified nucleotides and about seventeen 2′-O-methyl modified nucleotides, and wherein the antisense strand comprises about six 2′-fluoro modified nucleotides and about seventeen 2′-O-methyl modified nucleotides.

In some aspects, described herein is a specific modification pattern, wherein the sense strand comprises four 2′-fluoro modified nucleotides, seventeen 2′-O-methyl modified nucleotides, and no other nucleotides, and wherein the antisense strand comprises six 2′-fluoro modified nucleotides and seventeen 2′-O-methyl modified nucleotides and no other nucleotides.

In some aspects, described herein is a specific modification pattern, wherein the sense strand comprises ‘5-nnnnnnNfnNfNfNfnnnnnnnnnn-3’, wherein the antisense strand comprises ‘5-nNfnnnNfnNfNfnnnnNfnNfnnnnnnn-3’, wherein “Nf” stands for a 2′-fluoro modified nucleotide, and wherein “n” stands for a 2′-O-methyl modified nucleotide.

In some aspects, described herein is a specific modification pattern, wherein the sense strand comprises ‘5-nnnnnnNfnNfNfNfnnnnnnnnnn-3’, wherein the antisense strand comprises ‘5-nNfnnnNfnNfNfnnnnNfnNfnnnnnnn-3’, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises ‘5-nnnnnnNfnNfNfNfnnnnnnnnn-3’, wherein the antisense strand comprises ‘5-nNfnnnNfnNfNfnnnnNfnNfnnnnnnn-3’, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type II in Table 4.

In some aspects, the polynucleotide molecule provided herein comprises a sense strand comprising one of SEQ ID NOs: 1, 3, 5, 7, 9, 25-127, and 437-539, and/or an antisense strand comprising one of SEQ ID NOs: 2, 4, 6, 8, 10, and 231-333, and wherein the sense and/or antisense strand is modified in Type II modification pattern specified in Table 4. In other aspects, the polynucleotide molecule provided herein comprises a sense strand comprising one of SEQ ID NOs: 1, 3, 5, 7, and 9, and/or an antisense strand comprising one of SEQ ID NOs: 2, 4, 6, 8, and 10, and wherein the sense and/or antisense strand is modified in Type II modification pattern specified in Table 4.

In some aspects, described herein is a specific modification pattern, wherein the sense strand comprises about two 2′-fluoro modified nucleotides and about nineteen 2′-O-methyl modified nucleotides, and wherein the antisense strand comprises about three 2′-fluoro modified nucleotides and about twenty 2′-O-methyl modified nucleotides.

In some aspects, described herein is a specific modification pattern, wherein the sense strand comprises two 2′-fluoro modified nucleotides, nineteen 2′-O-methyl modified nucleotides, and no other nucleotides, and wherein the antisense strand comprises three 2′-fluoro modified nucleotides and twenty 2′-O-methyl modified nucleotides and no other nucleotides.

In some aspects, described herein is a specific modification pattern, wherein the sense strand comprises ‘5-nnnnnnnnNfnNfnnnnnnnnnn-3’, wherein the antisense strand comprises ‘5-nNfnnnnnnnnnNfnNfnnnnnnnnn-3’, wherein “Nf” stands for a 2′-fluoro modified nucleotide, and wherein “n” stands for a 2′-O-methyl modified nucleotide.

In some aspects, described herein is a specific modification pattern, wherein the sense strand comprises ‘5-nnnnnnnnNfnNfnnnnnnnnnn-3’, wherein the antisense strand comprises ‘5-nNfnnnnnnnnnNfnNfnnnnnnnnn-3’, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises ‘5-nnnnnnnnNfnNfnnnnnnnnnn-3’, wherein the antisense strand comprises ‘5-nNfnnnnnnnnnNfnNfnnnnnnnnn-3’, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type III in Table 4.

In some aspects, the polynucleotide molecule provided herein comprises a sense strand from one of SEQ ID NOs: 1, 3, 5, 7, 9, 25-127, and 437-539, an antisense strand comprises one of SEQ ID NOs: 2, 4, 6, 8, 10, and 231-333, and wherein the sense and/or antisense strand is modified in Type III modification pattern specified in Table 4. In other aspects, the polynucleotide molecule provided herein comprises a sense strand from one of SEQ ID NOs: 1, 3, 5, 7, and 9, an antisense strand comprises one of SEQ ID NOs: 2, 4, 6, 8, and 10, and wherein the sense and/or antisense strand is modified in Type III modification pattern specified in Table 4.

In some aspects, described herein is a specific modification pattern, wherein the sense strand comprises about three 2′-fluoro modified nucleotides and about eighteen 2′-O-methyl modified nucleotides, and wherein the antisense strand comprises about four 2′-fluoro modified nucleotides and about nineteen 2′-O-methyl modified nucleotides.

In some aspects, described herein is a specific modification pattern, wherein the sense strand comprises three 2′-fluoro modified nucleotides, eighteen 2′-O-methyl modified nucleotides, and no other nucleotides, and wherein the antisense strand comprises four 2′-fluoro modified nucleotides and nineteen 2′-O-methyl modified nucleotides and no other nucleotides.

In some aspects, described herein is a specific modification pattern, wherein the sense strand comprises ‘5-nnnnnnNfnNfnNfnnnnnnnnnn-3’, wherein the antisense strand comprises ‘5-nNfnnnnnnnnnNfnNfnNfnnnnnnn-3’, wherein “Nf” stands for a 2′-fluoro modified nucleotide, and wherein “n” stands for a 2′-O-methyl modified nucleotide.

In some aspects, described herein is a specific modification pattern, wherein the sense strand comprises ‘5-nnnnnnNfnNfnNfnnnnnnnnnn-3’, wherein the antisense strand comprises ‘5-nNfnnnnnnnnnNfnNfnNfnnnnnnn-3’, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises ‘5-nnnnnnNfnNfnNfnnnnnnnnnn-3’, wherein the antisense strand comprises ‘5-nNfnnnnnnnnnNfnNfnNfnnnnnnn-3’, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type IV in Table 4.

In some aspects, the polynucleotide molecule provided herein comprises a sense strand comprising one of SEQ ID NOs: 1, 3, 5, 7, 9, 25-127, and 437-539, and/or an antisense strand comprising one of SEQ ID NOs: 2, 4, 6, 8, 10, and 231-333, and wherein the sense and/or antisense strand is modified in Type IV modification pattern specified in Table 4. In other aspects, the polynucleotide molecule provided herein comprises a sense strand comprising one of SEQ ID NOs: 1, 3, 5, 7, and 9, and/or an antisense strand comprising one of SEQ ID NOs: 2, 4, 6, 8, and 10, and wherein the sense and/or antisense strand is modified in Type IV modification pattern specified in Table 4.

A polynucleic acid molecule for modulating expression of PCSK9 gene, wherein polynucleic acid molecule comprises an antisense strand comprising the nucleotide sequence of usUfsacaaaagcaAfaAfcAfggucusasg (SEQ ID NO: 14) and a sense strand comprising the nucleotide sequence of asgsaccuGfuUfuUfgcuuuuguaa (SEQ ID NO: 13), wherein “Nf” stands for a 2′-fluoro modified nucleotide, “n” stands for a 2′-O-methyl modified nucleotide, and “s” refers to 3′-phosphorothioate.

A polynucleic acid molecule for modulating expression of PCSK9 gene, wherein polynucleic acid molecule comprises an antisense strand comprising the nucleotide sequence of usUfsucaaguuacAfaAfaGfcaaaascsa (SEQ ID NO: 16) and a sense strand comprising the nucleotide sequence of ususuugcUfuUfuGfuaacuugaaa (SEQ ID NO: 15), wherein “Nf” stands for a 2′-fluoro modified nucleotide, “n” stands for a 2′-O-methyl modified nucleotide, and “s” refers to 3′-phosphorothioate.

A polynucleic acid molecule for modulating expression of PCSK9 gene, wherein polynucleic acid molecule comprises an antisense strand comprising the nucleotide sequence of asAfsuaucuucaaGfuUfaCfaaaagscsa (SEQ ID NO: 18) and a sense strand comprising the nucleotide sequence of csusuuugUfaAfcUfugaagauauu (SEQ ID NO: 17), wherein “Nf” stands for a 2′-fluoro modified nucleotide, “n” stands for a 2′-O-methyl modified nucleotide, and “s” refers to 3′-phosphorothioate.

A polynucleic acid molecule for modulating expression of PCSK9 gene, wherein polynucleic acid molecule comprises an antisense strand comprising the nucleotide sequence of asUfsuaauaaaaaUfgCfuAfcaaaascsc (SEQ ID NO: 20) and a sense strand comprising the nucleotide sequence of ususuuguAfgCfaUfuuuuauuaau (SEQ ID NO: 19), wherein “Nf” stands for a 2′-fluoro modified nucleotide, “n” stands for a 2′-O-methyl modified nucleotide, and “s” refers to 3′-phosphorothioate.

A polynucleic acid molecule for modulating expression of PCSK9 gene, wherein polynucleic acid molecule comprises an antisense strand comprising the nucleotide sequence of asUfsauuaauaaaAfaUfgCfuacaasasa (SEQ ID NO: 22) and a sense strand comprising the nucleotide sequence of ususguagCfaUfuUfuuauuaauau (SEQ ID NO: 21), wherein “Nf” stands for a 2′-fluoro modified nucleotide, “n” stands for a 2′-O-methyl modified nucleotide, and “s” refers to 3′-phosphorothioate.

### Conjugation

### Binding Moiety

In certain aspects, the polynucleotide molecule described herein is coupled or conjugated with one or more targeting moieties to form a polynucleotide-targeting moiety conjugate molecule. In some instances, a targeting moiety is selected based on its ability to target the conjugate molecule described herein to a desired cell population, tissue, or an organ selectively or preferably. In some instances, the targeting moiety targets the cell, tissue, or an organ that expresses the corresponding binding partner (e.g., either the corresponding receptor or ligand) of the targeting moiety. For example, the polynucleotide molecule described herein could be targeted to hepatocytes expressing asialoglycoprotein (ASGP-R) by selecting a targeting moiety containing N-acetyl galactosamine (GalNAc) as the targeting moiety. A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

The polynucleotide molecule described herein, thus, may include one or more targeting moieties selected from the group consisting of intracellular targeting moieties, extracellular targeting moieties, and combinations thereof. Thus, the inclusion of one or more targeting moieties (e.g., extracellular targeting moieties including targeting moieties independently selected from the group consisting of folate, mannose, N-acetyl galactosamine, and prostate specific membrane antigen) and one or more intracellular targeting moiety (e.g., a moiety targeting endoplasmic reticulum, Golgi apparatus, nucleus, or mitochondria) in the polynucleotide molecule described herein can facilitate the delivery of the polynucleotides to a specific site within the specific cell population. In some aspects, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944, 1987; Taylor et. al. J. Biol. Chem. 265:12156-62, 1990).

Some of the targeting moieties are described herein. In some aspects, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (SlPR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-Ira, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, and fragments thereof. In further aspects, the targeting moiety contains erythroblastic leukemia viral oncogene homolog (ErbB) receptor (e.g., ErbB1 receptor; ErbB2 receptor; ErbB3 receptor; and ErbB4 receptor). In some aspects, the targeting moiety contains one or more (e.g., from 1 to 6) N-acetyl galactosamines (GalNAc). In certain aspects, the targeting moiety contains one or more (e.g., from 1 to 6) mannoses. In other aspects, the targeting moiety contains a folate ligand. The folate ligand has the structure:

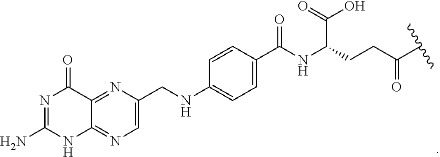 [SMILES: *C(=O)CC[C@H](NC(=O)c1ccc(NCc2cnc3[nH]c(N)nc(=O)c3n2)cc1)C(=O)O]

Certain targeting moieties may include bombesin, gastrin, gastrin-releasing peptide, tumor growth factors (TGF) (e.g., TGF-α or TGF-β), or vaccinia virus growth factor (VVGF). Non-peptidyl targeting moieties can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according techniques known in the art. Typically, however, the antibody is of human, murine, or rabbit origin. In addition, the antibody may be polyclonal or monoclonal, but is typically monoclonal. Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, single domain antibody, ScFv, or other antigen-binding fragments. Fc fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J. Immunological Methods, 56:235, 1983.

Other peptides for use as a targeting auxiliary moiety in polynucleotide molecule described herein can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

One or more (e.g., from 1 to 6) targeting moieties can be linked to MOIETY or to X2 in formula (V′, V″, or V′″) through -LinkA-.

In some aspects, the targeting moiety includes one or more (e.g., from 1 to 6 or from 1 to 3) asialoglycoprotein receptor ligands (e.g., GalNAc). In some aspects, an asialoglycoprotein receptor ligand (e.g., GalNAc) ligand is attached to -LinkA- through an anomeric carbon (e.g., where the anomeric carbon is the carbon atom in an acetal or a hemiaminal). In some aspects, an asialoglycoprotein receptor ligand (e.g., GalNAc) comprises an anomeric carbon bonded to trivalent, tetravalent linker, pentavalent, or hexavalent linker, wherein the anomeric carbon is part of a hemiaminal group. An asialoglycoprotein receptor ligand (e.g., GalNAc) attached to a linker through a hemiaminal may produce a hybridized polynucleotide construct having superior efficacy in gene silencing as compared to hybridized polynucleotide constructs having the asialoglycoprotein receptor ligand (e.g., GalNAc) attached to a linker through an acetal.

In some aspects, the linker and three asialoglycoprotein receptor targeting moieties, each of which comprises GalNAc, are as shown in Formula (V). In some instances, the conjugate described herein only comprises one asialoglycoprotein receptor targeting moiety, so the conjugate comprises a structure of Formula (V) with any two of the targeting moieties removed. In some instances, the conjugate described herein only comprises two asialoglycoprotein receptor targeting moieties, so the conjugate described herein comprises a structure of Formula (V) with any one of the targeting moieties removed.

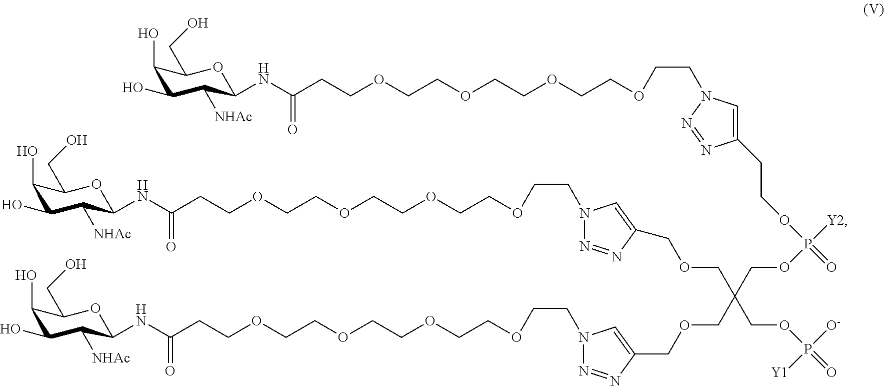

wherein one of Y1 and Y2 is nucleotide, or wherein both Y1 and Y2 are nucleotides and Y1 and Y2 are consecutive or neighboring nucleotides from the polynucleic acid molecule described herein.

In some aspects, the linker and the targeting moieties described herein are conjugated to 3′ end of the sense strand (e.g., as shown in Formula (V′)). In some aspects, the linker and the targeting moieties described herein are conjugated to 5′ end of the sense strand (e.g., as shown in Formula (V″) or (V′″)). In some aspects, the linker and the targeting moieties described herein are conjugated to 3′ end of the antisense strand (e.g., as shown in Formula (V′)). In some aspects, the linker and the targeting moieties described herein are conjugated to 5′ end of the antisense strand (e.g., as shown in Formula (V″) or (V′″)).

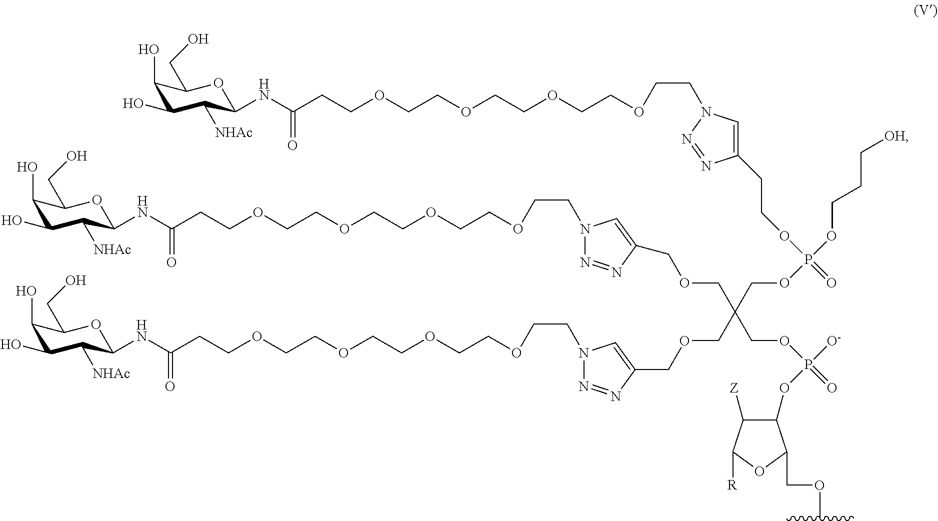

wherein Z in formula (V′) corresponds to one of the sugar modifications described herein (e.g., —H, —OH, —O-Methyl, —F, or —O-methoxyethyl), and R in formula (V′) is adenine, uracil, guanine, cytosine, thymine, abasic, or others.

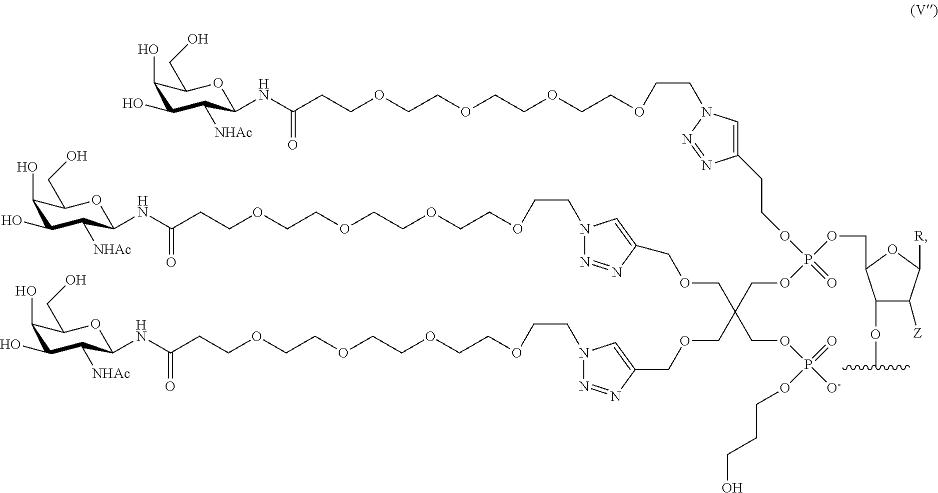

wherein Z in formula (V″) is a moiety that corresponds to one of the sugar modifications described herein (e.g., —H, —OH, —O-Methyl, —F, or —O-methoxyethyl) and R in formula (V″) is adenine, uracil, guanine, cytosine, thymine, abasic, or others.

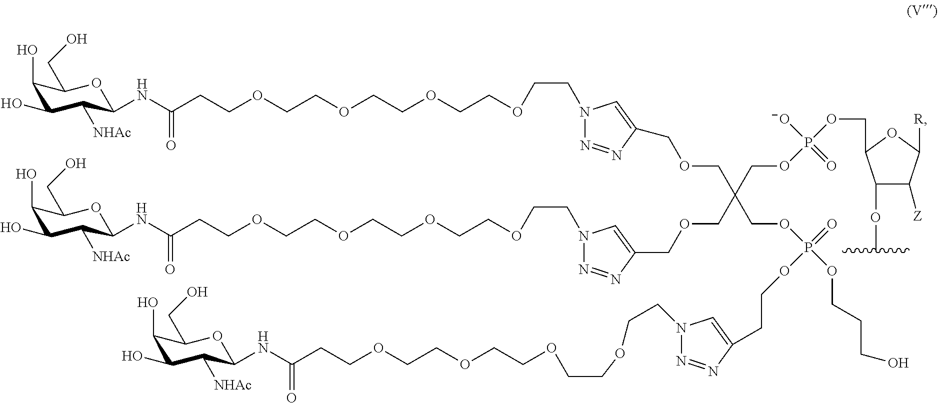

wherein Z in formula (V′″) is a moiety that corresponds to one of the sugar modifications described herein (e.g., —H, —OH, —O-Methyl, —F, or —O-methoxyethyl) and R in formula (V′″) is adenine, uracil, guanine, cytosine, thymine, abasic, or others.

In some instances, the 3′ end of passenger/sense strand from Table 1, Table 2, or Table 3 is conjugated with X2-GalNAc (see Formula (V) or (V′)). In some instances, the 5′ end of passenger/sense strand from Table 1, Table 2, or Table 3 is conjugated with X2-GalNAc (see Formula (V), (V″), or (V′″)). In some instances, a nucleic acid within passenger/sense strand (not at the 5′ or 3′ end) from Table 1, Table 2, or Table 3 is conjugated with X2-GalNAc (see Formula (V)). In some instances, the 3′ end of guide/antisense strand from Table 1, Table 2, or Table 3 is conjugated with X2-GalNAc (see Formula (V) or (V′)). In some instances, the 5′ end of guide/antisense strand from Table 1, Table 2, or Table 3 is conjugated with X2-GalNAc (see Formula (V), (V″), or (V′″)). In some instances, a nucleic acid within guide/antisense strand (not at the 5′ or 3′ end) from Table 1, Table 2, or Table 3 is conjugated with X2-GalNAc (see Formula (V)).

One or more endosomal escape moieties (e.g., from 1 to 6 or from 1 to 3) can be attached to a polynucleotide construct or a hybridized polynucleotide construct disclosed herein as an auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in WO 2015/188197; the disclosure of these endosomal escape moieties is incorporated by reference herein.

One or more endosomal escape moieties (e.g., from 1 to 6 or from 1 to 3) can be attached to a MOIETY or X2 in formula (V′, V″, or V′″) through -LinkA-, as described herein.

One or more cell penetrating peptides (CPP) (e.g., from 1 to 6 or from 1 to 3) can be attached to a polynucleotide construct or a hybridized polynucleotide construct disclosed herein as an auxiliary moiety. The CPP can be linked to the hybridized polynucleotide bioreversibly through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one embodiment, a CPP useful in the methods and compositions as described herein includes a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP described herein may be a naturally occurring peptide or a synthetic peptide.

One or more cell penetrating peptides (e.g., from 1 to 6 or from 1 to 3) can be attached to a MOIETY or X2 in formula (V′, V″, or V′″) through -LinkA-, as described herein.

The polynucleotide constructs and the hybridized polynucleotide constructs disclosed herein can also include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

One or more polymers (e.g., from 1 to 6 or from 1 to 3) can be attached to a MOIETY or X2 in formula (V′, V″, or V′″) through -LinkA-, as described herein.

### Conjugation Linkers

In some aspects, the polynucleic acid molecules described herein comprises a sense or antisense strand bonded to at least one group of formula (I)

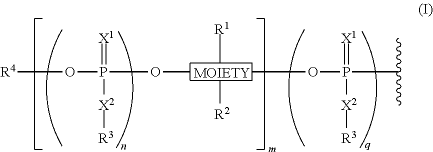

or a salt thereof, or a stereoisomer thereof,
where
each X¹ is independently O or S;
each X² is independently O, S, NH, or a bond;
MOIETY is optionally substituted C₂₋₁₀ alkane-tetrayl or a group -M¹-M²-M³-, wherein each M¹ and each M³ is independently absent or optionally substituted C₁₋₆ alkylene, and M² is optionally substituted C₃₋₉ heterocycle-tetrayl, optionally substituted C₆₋₁₀ arene-tetrayl, or optionally substituted C₃₋₈ cycloalkane-tetrayl;
each R¹ and each R² is independently H, optionally substituted C₁₋₁₆ alkyl, optionally substituted C₂₋₁₆ heteroalkyl, a conjugation moiety, or -LinkA(-T)ₚ, provided that at least one R¹ or at least one R² is a conjugation moiety or -LinkA(-T)ₚ;
each R³ is independently H, optionally substituted C₁₋₁₆ alkyl, optionally substituted C₂₋₁₆ heteroalkyl, optionally substituted C₂₋₁₆ alkenyl, optionally substituted C₂₋₁₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, a conjugation moiety, or -LinkA(-T)ₚ;
R⁴ is H, optionally substituted C₁₋₆ alkyl, -LinkA(-T)ₚ, or -Sol;
each LinkA is independently a multivalent linker (e.g., including —C(O)—N(H)— (e.g., at least one multivalent linker including —C(O)—N(H)— bonded to T));
each T is independently an auxiliary moiety;
Sol is solid support;
m is an integer from 1 to 6;
each n is independently 0 or 1;
each p is independently an integer from 1 to 6; and
q is an integer from 0 to 3.

The at least one group of formula (I) may be bonded to a 5′-terminus, 3′-terminus, internucleoside phosphate, internucleoside phosphorothioate, or internucleoside phosphorodithioate of the polynucleotide. When the at least one group of formula (I) is bonded to the internucleoside phosphate, internucleoside phosphorothioate, or internucleoside phosphorodithioate, q is 0. The polynucleotide construct contains no more than one Sol.

Group -LinkA- can include from 0 to 3 multivalent monomers (e.g., optionally substituted C1-6 alkane-triyl, optionally substituted C1-6 alkane-tetrayl, or trivalent nitrogen atom) and one or more divalent monomers (e.g., from 1 to 40), where each divalent monomer is independently optionally substituted C1-6 alkylene; optionally substituted C2-6 alkenylene; optionally substituted C2-6 alkynylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, wherein m is 0, 1, or 2. In some aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, where m is 0, 1, or 2 (e.g., m is 2). In certain aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; optionally substituted N; O; or S(O)m, where m is 0, 1, or 2 (e.g., m is 2). The non-bioreversible linker connecting the auxiliary moiety to the conjugating moiety or to the reaction product thereof can include from 2 to 500 (e.g., from 2 to 300 or from 2 to 200) of such monomers. Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some aspects, the non-bioreversible linker includes polyethylene oxide (e.g., poly(ethylene oxide) having a molecular weight of less than 1 kDa).

Group -LinkA(-T)p in formula (I) may be prepared by a process described in the sections below. In some instances, -LinkA(-T)p is of formula (II):

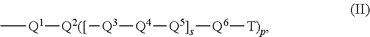

where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C}- where Q^{C} is optionally substituted C₂₋₁₂ heteroalkylene (e.g., a heteroalkylene containing —C(O)—N(H)—, —N(H)—C(O)—, —S(O)₂-N(H)—, or —N(H)—S(O)₂—), optionally substituted C₁₋₁₂ thioheterocyclylene (e.g.,

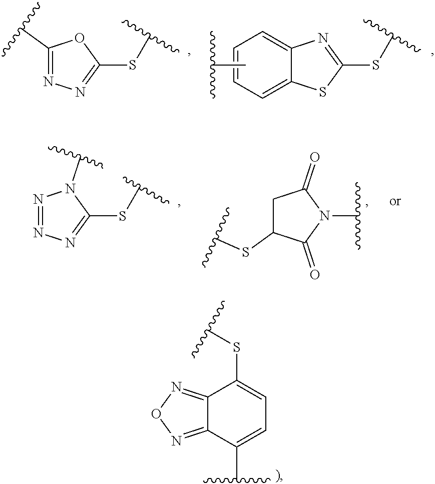

optionally substituted C₁₋₁₂ heterocyclylene (e.g., 1,2,3-triazole-1,4-diyl or

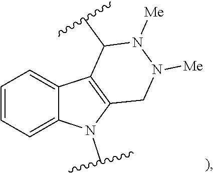 [SMILES: *C1c2c(n(*)c3ccccc23)CN(C)N1C]

cyclobut-3-ene-1,2-dione-3,4-diyl, or pyrid-2-yl hydrazone);
Q² is a linear group (e.g., [-Q³-Q⁴-Q]ₛ-), if p is 1, or a branched group (e.g., [-Q³-Q⁴-Q⁵]ₛ-Q⁷([-Q³-Q⁴-Q⁵]ₛ-(Q⁷)ₚ₁)ₚ₂, where p1 is 0 or 1, p2 is 0, 1, 2, or 3), if p is an integer from 2 to 6;
each Q³ and each Q⁶ is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, or —C(O)—CH(R^{a})—NH—;
each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each R^{a} is independently H or an amino acid side chain;
provided that at least one of Q³, Q⁴, and Q⁵ is present.

In some aspects, each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene. In certain aspects, s is 1, 2, 3, 4, 5, 6, 7, 8, 9, 10, 11, 12, 13, 14, 15, 16, 17, 18, 19, or 20.

Thus, in formula (II), LinkA may include a single branching point, if each p1 is 0, or multiple branching points, if at least one p1 is 1.

In formula (II), Q¹ may be —O-Q^{L}-Q^{C}-, where Q^{L} is optionally substituted C₂₋₁₂ heteroalkylene, optionally substituted C₁₋₁₂ alkylene, or -(optionally substituted C1-6 alkylene)-(optionally substituted C₆₋₁₀ arylene)-. In some aspects, Q^{L} is optionally substituted C₂₋₁₂ heteroalkylene or optionally substituted C₁₋₁₂ alkylene. In formula (II) Q^{C} may be:

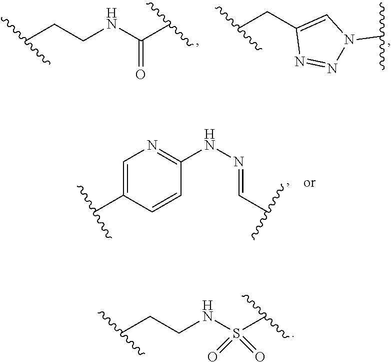 [SMILES: *CCNC(*)=O ; *Cc1cn(*)nn1 ; */C=N/Nc1ccc(*)cn1 ; *CCNS(*)(=O)=O]

In formula (II), Q² may be a linear group of formula [-Q³-Q⁴-Q⁵]ₛ- where Q³, Q⁴, and Q⁵ are as defined for formula (II). Alternatively, Q² may be a branched group [-Q³-Q⁴-Q⁵]ₛ-Q⁷([-Q³-Q⁴-Q⁵]ₛ-(Q⁷)ₚ₁)ₚ₂, where each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;
where
p1 is 0 or 1;
p2 is 0, 1, 2, or 3;
where,
   when p1 is 0, LinkA is a trivalent or tetravalent linker, and,
   when p1 is 1, LinkA is a tetravalent, pentavalent, or hexavalent linker.

In certain aspects, p1 is 0.

In some aspects, Q⁷ is:

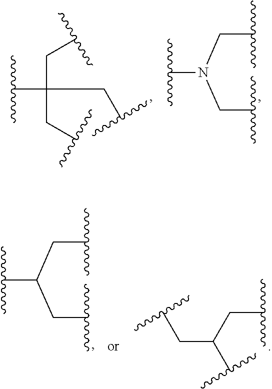 [SMILES: *CC(*)(C*)C* ; *CN(*)C* ; *CC(*)C* ; *CC(*)C*]

Compounds that may be used in the preparation of group -LinkA(-T)p in formula (I) are described herein as well as in WO 2015/188197. Non-limiting examples of -LinkA include:

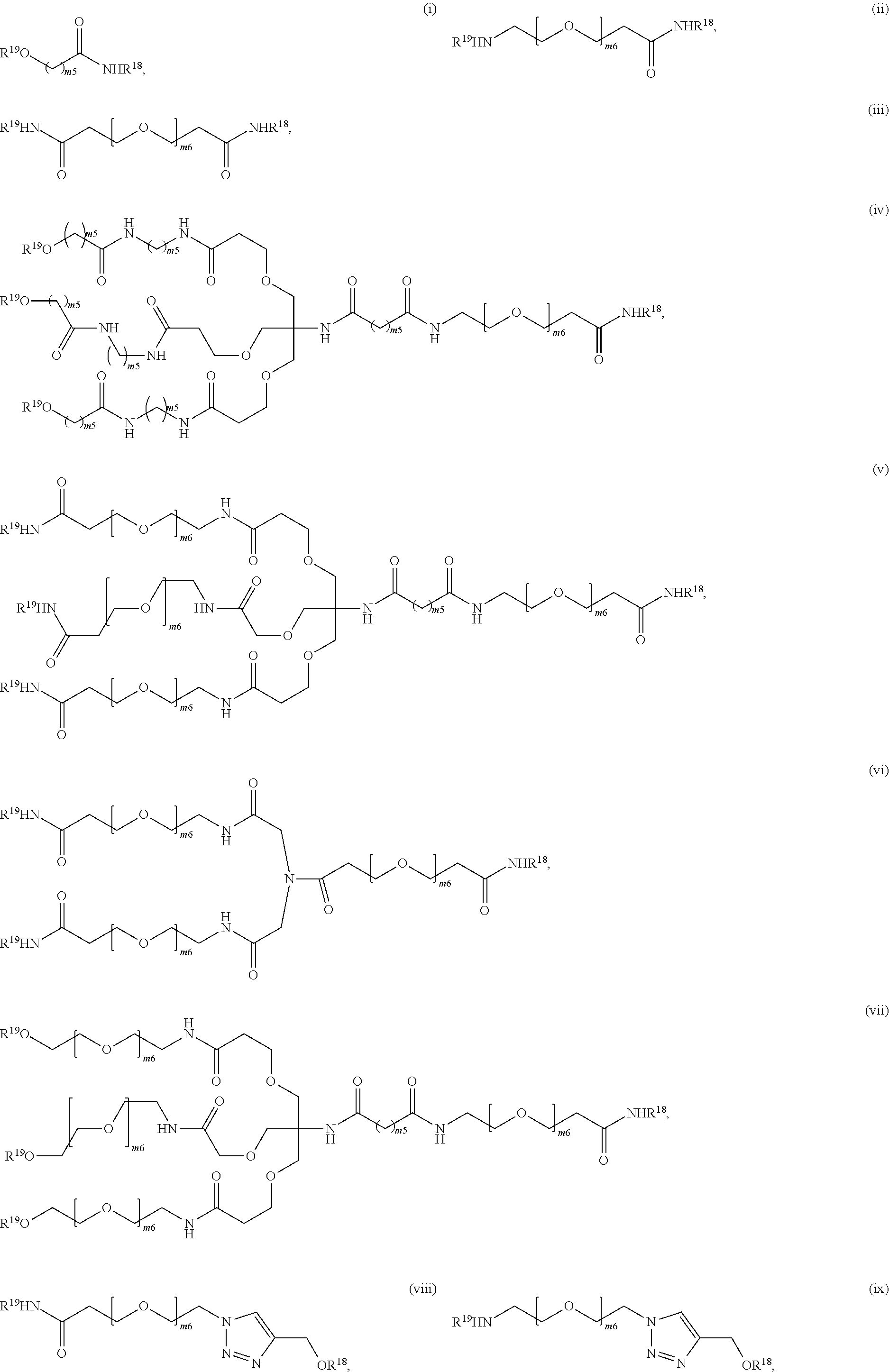

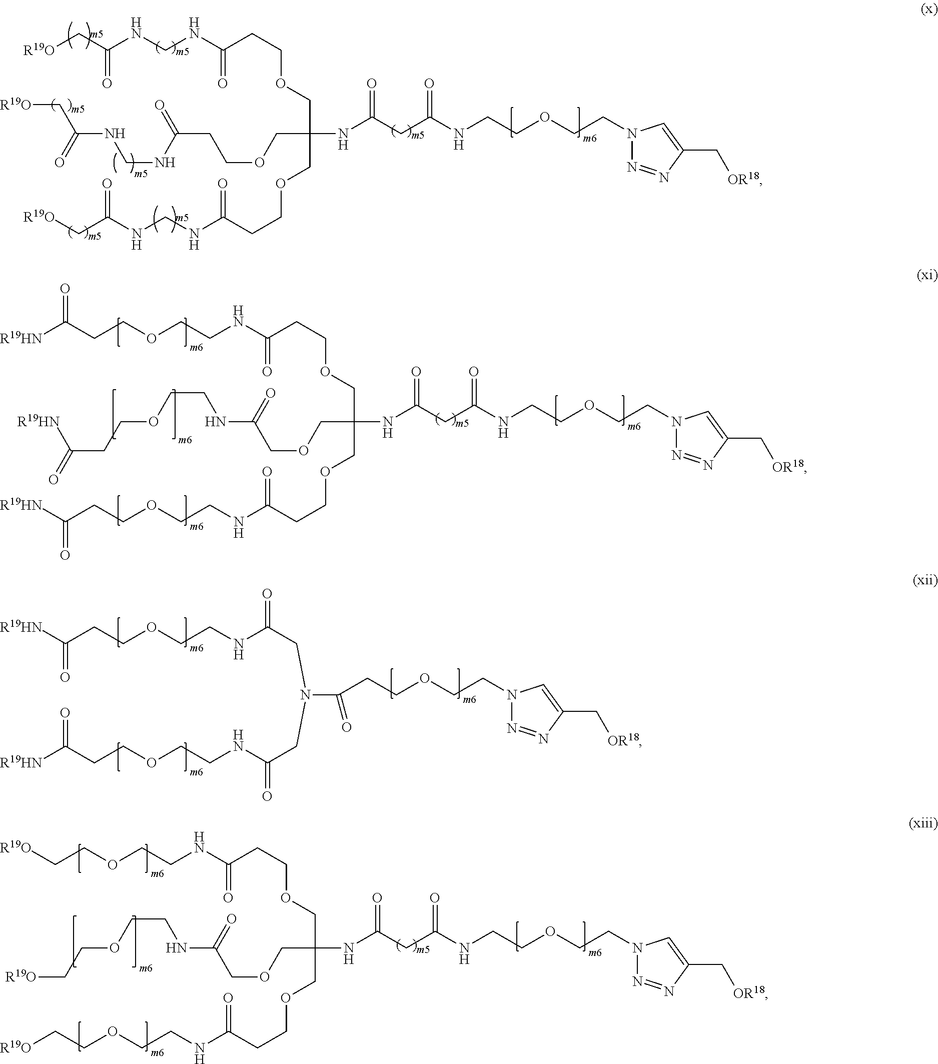

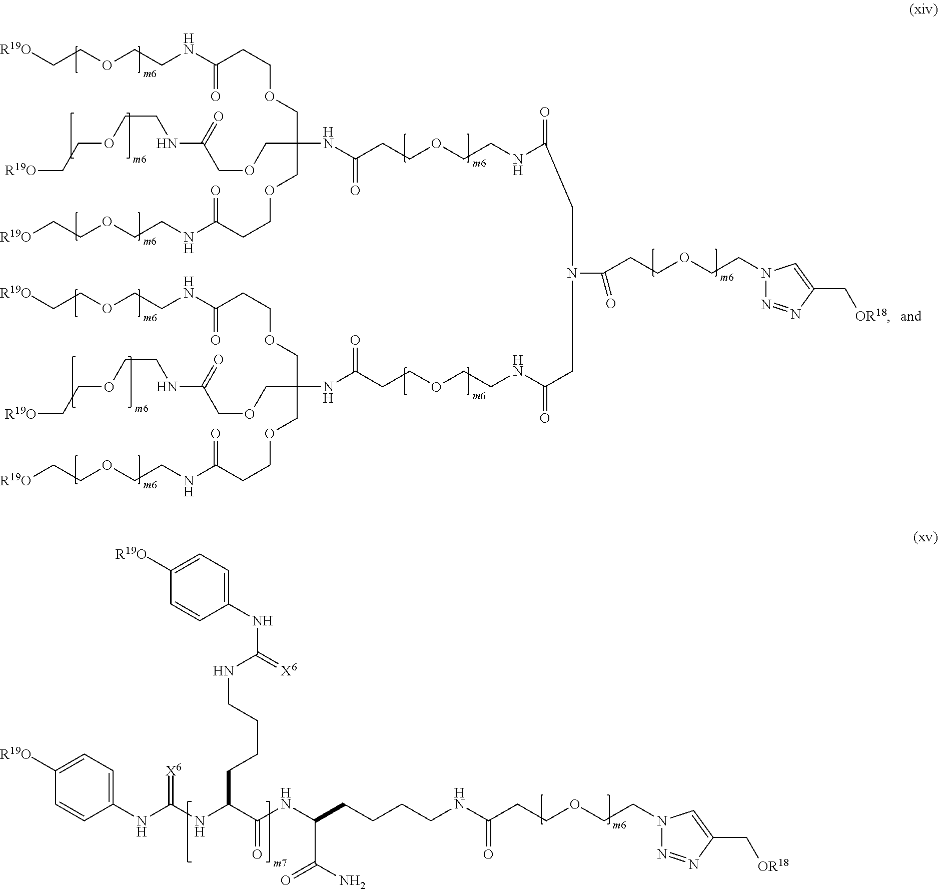

where
R¹⁸ is a bond to MOIETY,
each R¹⁹ is independently a bond to auxiliary moiety,
each m5 is independently an integer from 1 to 20,
each m6 is independently an integer from 1 to 10,
m7 is an integer from 1 to 6, and
each X⁶ is independently O or S.

In formula (II), when the conjugation linker is of formula [-Q³-Q⁴-Q⁵]ₛ-Q^{C}-, -Q²([-Q³-Q⁴-Q⁵]ₛ-Q⁶-T)ₚ may be.

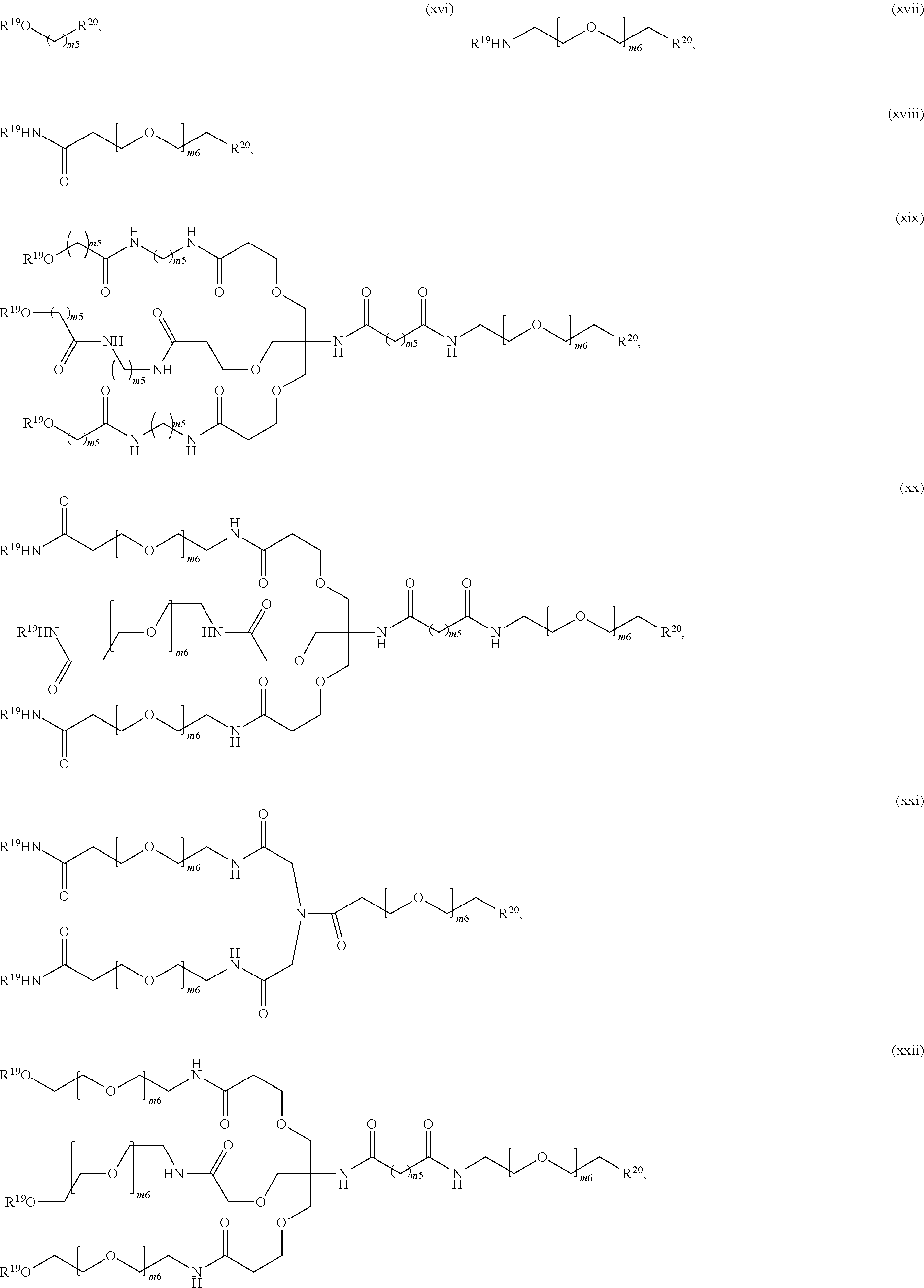

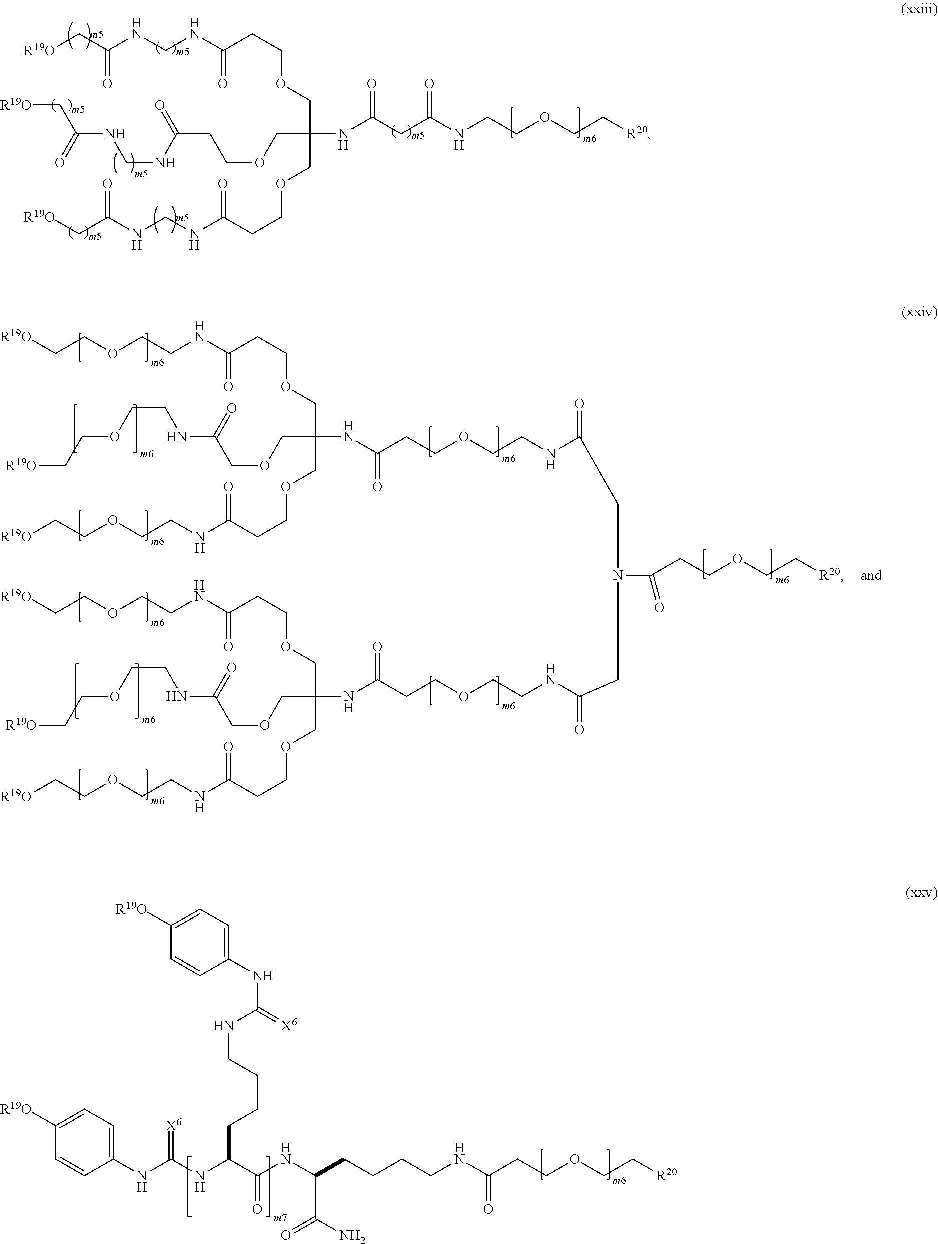

where
R²⁰ is a bond to Q^{C} in Q¹
each R¹⁹ is independently a bond to an auxiliary moiety,
each m5 is independently an integer from 1 to 20,
each m6 is independently an integer from 1 to 10,
m7 is an integer from 1 to 6, and
each X⁶ is independently O or S.

In some aspects, the linker described herein is cleavable. In some aspects, the linker described herein is non-cleavable.

In some aspects, the polynucleic acid molecule described herein comprises a sense or antisense strand bonded to at least one group of formula (IV),

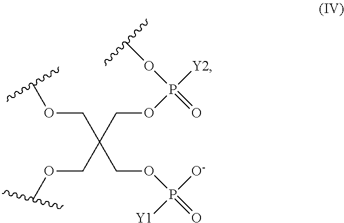

wherein at least one of Y1 or Y2 is a nucleotide from the polynucleic acid molecule.

In some instances, the Y1 is the last nucleotide on the 3′-terminus or the first nucleotide on the 5′-terminus of one of the strands of the polynucleic acid molecule. In some instances, the Y1 is the last nucleotide on the 3′-terminus or the first nucleotide on the 5′-terminus of the sense strand of the polynucleic acid molecule. In some instances, the Y1 is the last nucleotide on the 3′-terminus or the first nucleotide on the 5′-terminus of the sense strand of the polynucleic acid molecule, and the Y2 is a 3-hydroxy-propoxy group. In some instances, the Y2 is the first nucleotide on the 5′-terminus or the last nucleotide on the 3′-terminus of one of the strands of the polynucleic acid molecule. In some instances, the Y2 is the first nucleotide on the 5′-terminus or the last nucleotide on the 3′-terminus of the sense strand of the polynucleic acid molecule. In some instances, the Y2 is the first nucleotide on the 5′-terminus or the last nucleotide on the 3′-terminus of the sense strand of the polynucleic acid molecule, and the Y1 is a 3-hydroxy-propoxy group. In other instances, the Y1 and Y2 are two consecutive nucleotides in one of the strands of the polynucleic acid molecule.

In some aspects, the targeting moiety described herein is conjugated to 3′ end of the sense strand (e.g., formula (IV′)). In some aspects, the targeting moiety described herein is conjugated to 5′ end of the sense strand (e.g., formula (IV″) or (IV′″)). In some aspects, the targeting moiety described herein is conjugated to 3′ end of the antisense strand (e.g., formula (IV′)). In some aspects, the targeting moiety described herein is conjugated to 5′ end of the antisense strand (e.g., formula (IV″) or (IV′″)).

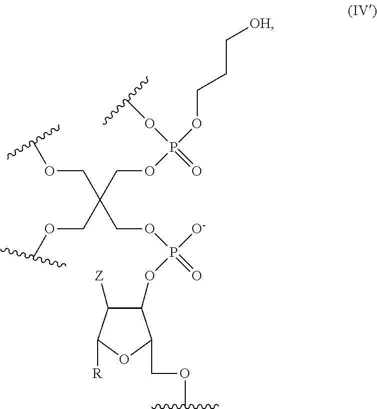

wherein Z in formula (IV′) is a moiety that corresponds to one of the sugar modifications described herein (e.g., —H, —OH, —O-Methyl, —F, or —O-methoxyethyl) and R in formula (IV′) is adenine, uracil, guanine, cytosine, thymine, abasic, or others.

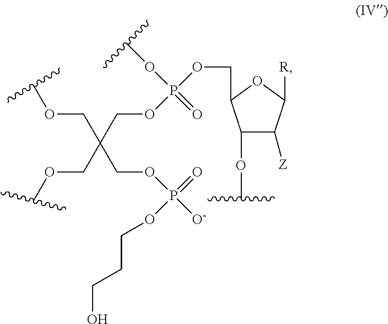

wherein Z in formula (IV″) is a moiety that corresponds to one of the sugar modifications described herein (e.g., —H, —OH, —O-Methyl, —F, or —O-methoxyethyl) and R in formula (IV″) is adenine, uracil, guanine, cytosine, thymine, abasic, or others.

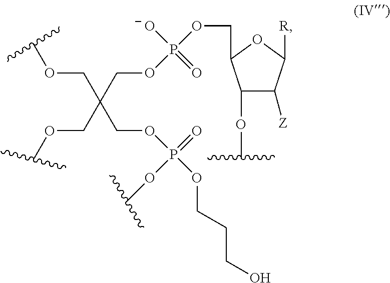

wherein Z in formula (IV′″) is a moiety that corresponds to one of the sugar modifications described herein (e.g., —H, —OH, —O-Methyl, —F, or —O-methoxyethyl) and R in formula (IV′″) is adenine, uracil, guanine, cytosine, thymine, abasic, or others.

### Pharmaceutical Compositions

Delivery of the polynucleotide molecules described herein can be achieved by contacting a cell with the construct using a variety of methods. In particular aspects, the polynucleotide molecule described herein is formulated with various excipients, vehicles, and carriers, as described more fully elsewhere herein.

A pharmaceutical composition described herein can be prepared to include a hybridized polynucleotide construct disclosed herein, into a form suitable for administration to a subject using carriers, excipients, and vehicles. Frequently used excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms are related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the polynucleotide molecule described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some specific aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcamitine, an acylcholine, or a monoglyceride, a diglyceride or a pharmaceutically acceptable salt thereof. In one specific aspect, the permeation enhancer is sodium caprate (C10). In some specific aspects, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some specific aspects, the permeation enhancer is polyoxyethylene-9-lauryl ether, or polyoxyethylene-20-cetyl ether.

For the polynucleotide molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

While the hybridized polynucleotide constructs described herein may not require the use of excipients for delivery to the target cell, the use of excipients may be advantageous in some aspects. Thus, for delivery to the target cell, the hybridized polynucleotide molecule described herein can non-covalently bind an excipient to form a complex. The excipient can be used to alter biodistribution after delivery, to enhance uptake, to increase half-life or stability of the strands in the hybridized polynucleotide constructs (e.g., improve nuclease resistance), and/or to increase targeting to a particular cell or tissue type.

Exemplary excipients include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition described herein in combination with a polynucleotide molecule described herein.

### Methods of Treatment

In some aspects, described herein is a method of modulating expression of PCSK9 gene in a subject, comprising: administering to the subject a polynucleic acid molecule described herein, a polynucleic acid molecule conjugate described herein, or a pharmaceutical composition described herein, thereby modulating the expression of PCSK9 gene in the subject.

In some specific aspects, the method described herein reduces expression of PCSK9 gene in a subject by about or at least 10% compared to a negative control. In some specific aspects, the method described herein reduces expression of PCSK9 gene in a subject by about or at least 20% compared to a negative control. In some specific aspects, the method described herein reduces expression of PCSK9 gene in a subject by about or at least 30% compared to a negative control. In some specific aspects, the method described herein reduces expression of PCSK9 gene in a subject by about or at least 40% compared to a negative control. In some specific aspects, the method described herein reduces expression of PCSK9 gene in a subject by about or at least 50% compared to a negative control. In some specific aspects, the method described herein reduces expression of PCSK9 gene in a subject by about or at least 60% compared to a negative control. In some specific aspects, the method described herein reduces expression of PCSK9 gene in a subject by about or at least 70% compared to a negative control. In some specific aspects, the method described herein reduces expression of PCSK9 gene in a subject by about or at least 80% compared to a negative control. In some specific aspects, the method described herein reduces expression of PCSK9 gene in a subject by about or at least 90% compared to a negative control. In some specific aspects, the method described herein reduces expression of PCSK9 gene in a subject by about 100% compared to a negative control.

In some specific aspects, the method described herein achieves an IC50 value of about 5 nM. In some specific aspects, the method described herein achieves an IC50 value of about 10 nM. In some specific aspects, the method described herein achieves an IC50 value of about 15 nM. In some specific aspects, the method described herein achieves an IC50 value of about 20 nM. In some specific aspects, the method described herein achieves an IC50 value of about 25 nM. In some specific aspects, the method described herein achieves an IC50 value of about 30 nM. In some specific aspects, the method described herein achieves an IC50 value of about 35 nM. In some specific aspects, the method described herein achieves an IC50 value of about 40 nM. In some specific aspects, the method described herein achieves an IC50 value of about 45 nM. In some specific aspects, the method described herein achieves an IC50 value of about 50 nM. In some specific aspects, the method described herein achieves an IC50 value of about 55 nM. In some specific aspects, the method described herein achieves an IC50 value of about 60 nM. In some specific aspects, the method described herein achieves an IC50 value of about 65 nM. In some specific aspects, the method described herein achieves an IC50 value of about 70 nM. In some specific aspects, the method described herein achieves an IC50 value of about 75 nM. In some specific aspects, the method described herein achieves an IC50 value of about 80 nM. In some specific aspects, the method described herein achieves an IC50 value of about 85 nM. In some specific aspects, the method described herein achieves an IC50 value of about 90 nM. In some specific aspects, the method described herein achieves an IC50 value of about 95 nM. In some specific aspects, the method described herein achieves an IC50 value of about 100 nM.

In some aspects, described herein is a method of modulating LDL in a subject in need thereof, comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein reduces the expression of PCSK9 gene in the subject.

In some specific aspects, the method described herein reduces LDL level in a subject by about or at least 10% compared to a negative control. In some specific aspects, the method described herein reduces LDL level in a subject by about or at least 20% compared to a negative control. In some specific aspects, the method described herein reduces LDL level in a subject by about or at least 30% compared to a negative control. In some specific aspects, the method described herein reduces LDL level in a subject by about or at least 40% compared to a negative control. In some specific aspects, the method described herein reduces LDL level in a subject by about or at least 50% compared to a negative control. In some specific aspects, the method described herein reduces LDL level in a subject by about or at least 60% compared to a negative control. In some specific aspects, the method described herein reduces LDL level in a subject by about or at least 70% compared to a negative control. In some specific aspects, the method described herein reduces LDL level in a subject by about or at least 80% compared to a negative control. In some specific aspects, the method described herein reduces LDL level in a subject by about or at least 90% compared to a negative control. In some specific aspects, the method described herein reduces LDL level in a subject by about 100% compared to a negative control.

In some aspects, the subject receiving the method described herein suffers from hypercholesterolemia. In some specific embodiment, the subject receiving the method described herein suffers from familial hypercholesterolemia. In other aspects, the subject receiving the method described herein suffers from other high cholesterol-associated diseases. In other aspects, the subject receiving the method described herein suffers from neuroinflammation. In other aspects, the subject receiving the method described herein suffers from Alzheimer's Disease. In other aspects, the subject receiving the method described herein suffers from AUD. In other aspects, the subject receiving the method described herein suffers from stroke.

In some aspects, described herein is a method of modulating cholesterol in a subject in need thereof, comprising: comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein reduces the expression of PCSK9 gene in the subject.

In some specific aspects, the method described herein reduces cholesterol level in a subject by about or at least 10% compared to a negative control. In some specific aspects, the method described herein reduces cholesterol level in a subject by about or at least 20% compared to a negative control. In some specific aspects, the method described herein reduces cholesterol level in a subject by about or at least 30% compared to a negative control. In some specific aspects, the method described herein reduces cholesterol level in a subject by about or at least 40% compared to a negative control. In some specific aspects, the method described herein reduces cholesterol level in a subject by about or at least 50% compared to a negative control. In some specific aspects, the method described herein reduces cholesterol level in a subject by about or at least 60% compared to a negative control. In some specific aspects, the method described herein reduces cholesterol level in a subject by about or at least 70% compared to a negative control. In some specific aspects, the method described herein reduces cholesterol level in a subject by about or at least 80% compared to a negative control. In some specific aspects, the method described herein reduces cholesterol level in a subject by about or at least 90% compared to a negative control. In some specific aspects, the method described herein reduces cholesterol level in a subject by about 100% compared to a negative control.

In some aspects, the subject receiving the method described herein suffers from hypercholesterolemia. In some specific embodiment, the subject receiving the method described herein suffers from familial hypercholesterolemia. In other aspects, the subject receiving the method described herein suffers from other high cholesterol-associated diseases. In other aspects, the subject receiving the method described herein suffers from neuroinflammation. In other aspects, the subject receiving the method described herein suffers from Alzheimer's Disease. In other aspects, the subject receiving the method described herein suffers from AUD. In other aspects, the subject receiving the method described herein suffers from stroke.

### EXAMPLES

These examples are provided for illustrative purposes only and not to limit the scope of the claims provided herein.

### Example 1—Testing PCSK9 siRNAs in Non-Human Primates

Five PCSK9 siRNAs (denoted as “SRS-001” to “SRS-005” from Table 1) were tested in a non-human primate study, with a known siRNA that targets PCSK9 (denoted as “SRS-006” in Table 1) as a benchmark.

TABLE 1
Sequences of siRNA Tested in Non-human Primates

| | | | | % | | | | | | | |
|---|---|---|---|---|---|---|---|---|---|---|---|
| | | | | remaining | | | % | | | | |
| | | | | after | | | remaining | | | | |
| | | | | siRNA | | | after | | | | |
| | | SEQ | | trans- | | | GalNAc- | | | SEQ | |
| siRNA | | ID | Unmodified Sequence | fection | 1 | 10 | siRNA | 100 | 500 | ID | |
| ID# | Strand | # | (5′-3′) | 100 pM | nM | nM | 10 nM | nM | Mn | # | Modified Sequence (5′-3′) |
| SRS- | sense | 1 | AGACCUGUUUUGCUUUUGUAA | 30 | 8 | 7 | 69 | 72 | 48 | 13 | asgsaccuGfuUfuUfgcuuuuguaa |
| 001 | | | | | | | | | | | |
| SRS- | anti- | 2 | UUACAAAAGCAAAACAGGUCUAG | | | | | | | 14 | usUfsacaaaagcaAfaAfcAfggucus |
| 001 | sense | | | | | | | | | | asg |
| SRS- | sense | 3 | UUUUGCUUUUGUAACUUGAAA | 25 | 8 | 4 | | | | 15 | ususuugcUfuUfuGfuaacuugaaa |
| 002 | | | | | | | | | | | |
| SRS- | anti- | 4 | UUUCAAGUUACAAAAGCAAAACA | | | | | | | 16 | usUfsucaaguuacAfaAfaGfcaaaas |
| 002 | sense | | | | | | | | | | csa |
| SRS | sense | 5 | CUUUUGUAACUUGAAGAUAUU | 14 | 7 | 3 | 63 | 36 | 35 | 17 | csusuuugUfaAfcUfugaagauauu |
| 003 | | | | | | | | | | | |
| SRS- | anti- | 6 | AAUAUCUUCAAGUUACAAAAGCA | | | | | | | 18 | asAfsuaucuucaaGfuUfaCfaaaags |
| 003 | sense | | | | | | | | | | csa |
| SRS- | sense | 7 | UUUUGUAGCAUUUUUAUUAAU | 27 | 8 | 6 | 68 | 63 | 68 | 19 | ususuuguAfgCfaUfuuuuauuaau |
| 004 | | | | | | | | | | | |
| SRS- | anti- | 8 | AUUAAUAAAAAUGCUACAAAACC | | | | | | | 20 | asUfsuaauaaaaaUfgCfuAfcaaaas |
| 004 | sense | | | | | | | | | | csc |
| SRS- | sense | 9 | UUGUAGCAUUUUUAUUAAUAU | 26 | 4 | 3 | | | | 21 | ususguagCfaUfuUfuuauuaauau |
| 005 | | | | | | | | | | | |
| SRS- | anti- | 10 | AUAUUAAUAAAAAUGCUACAAAA | | | | | | | 22 | asUfsauuaauaaaAfaUfgCfuacaas |
| 005 | sense | | | | | | | | | | asa |
| SRS- | sense | 11 | CUAGACCUGUTUUGCUUUUGU | | | | 83 | 64 | 52 | 23 | csusagacCfuGfudTuugcuuuugu |
| 006 | | | | | | | | | | | |
| SRS- | anti- | 12 | ACAAAAGCAAAACAGGUCUAGAA | | | | | | | 24 | asCfsaAfAfAfgCfaAfaAfcAfgG |
| 006 | sense | | | | | | | | | | fuCfuagsasa |

Note for the column of “modified sequence”:
“A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxy guanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate.

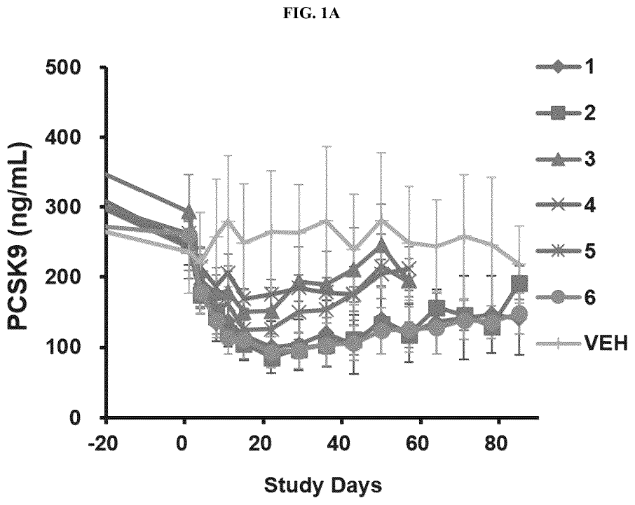

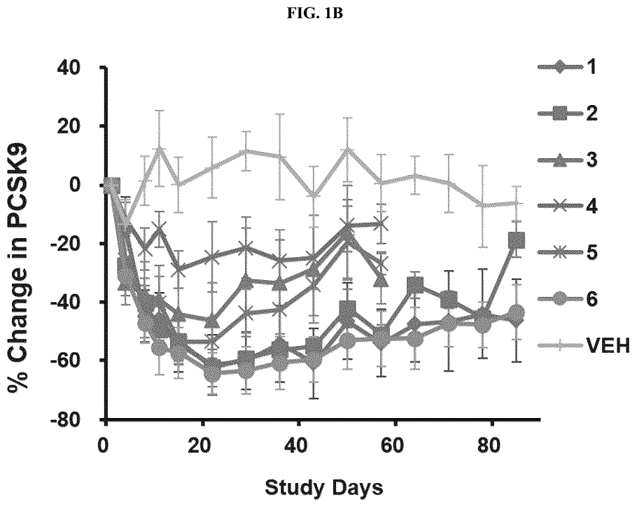

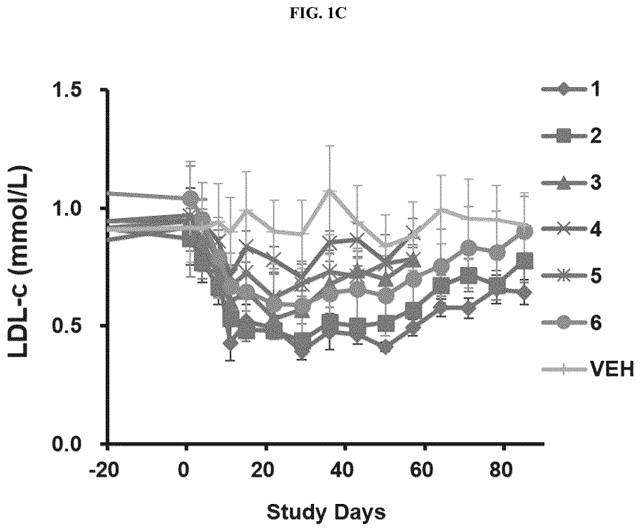

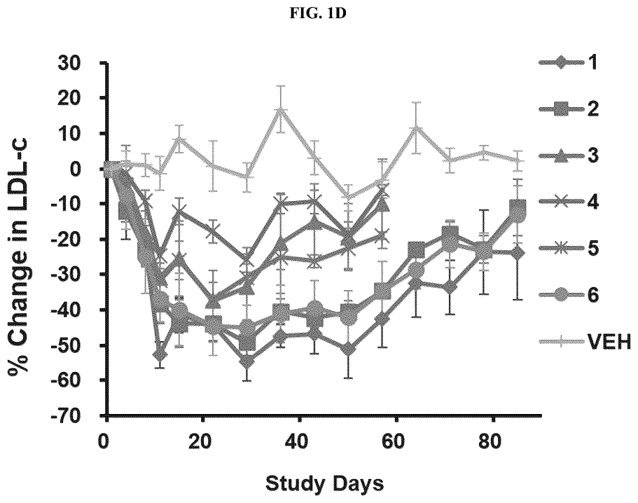

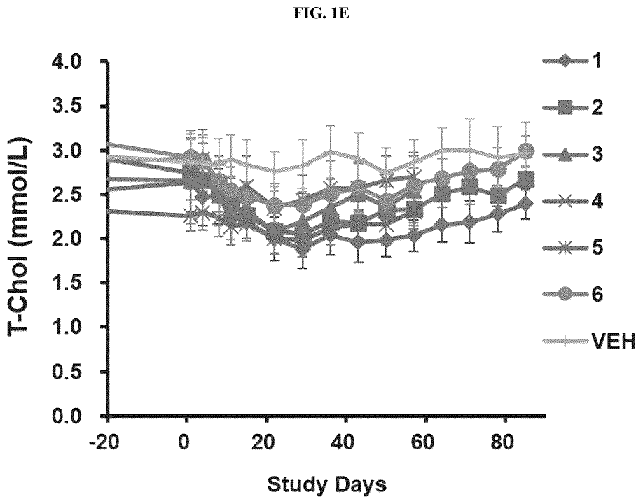

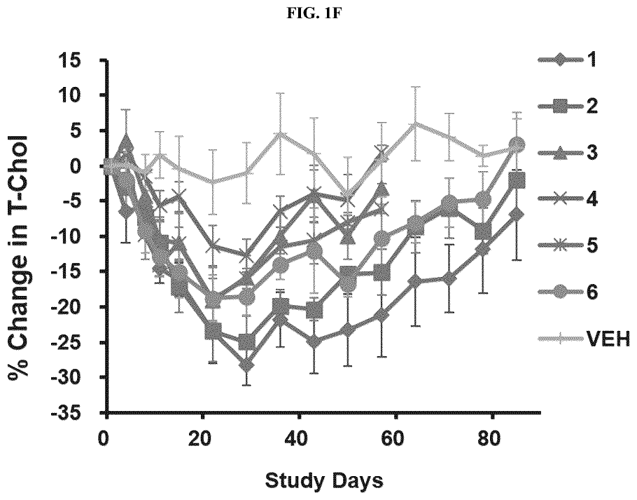

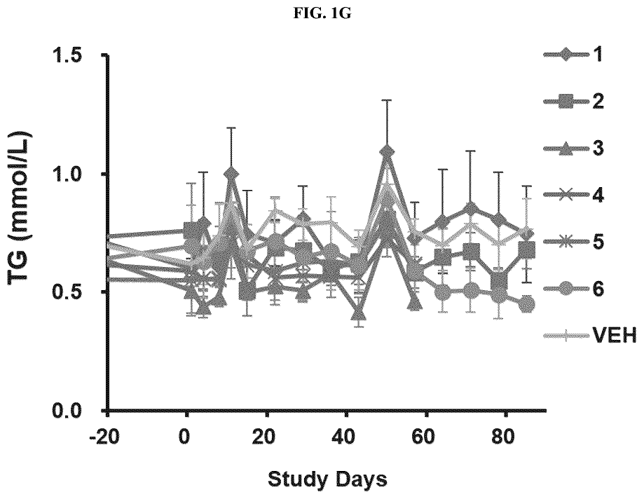

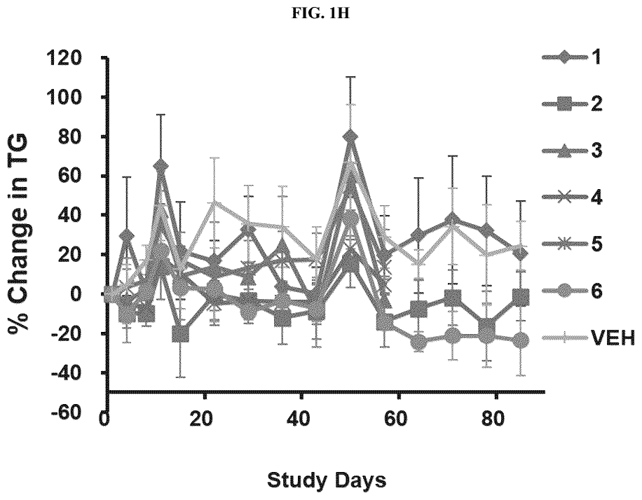

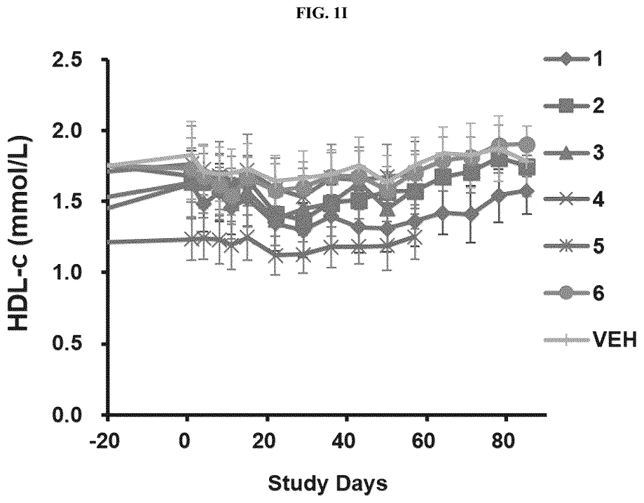

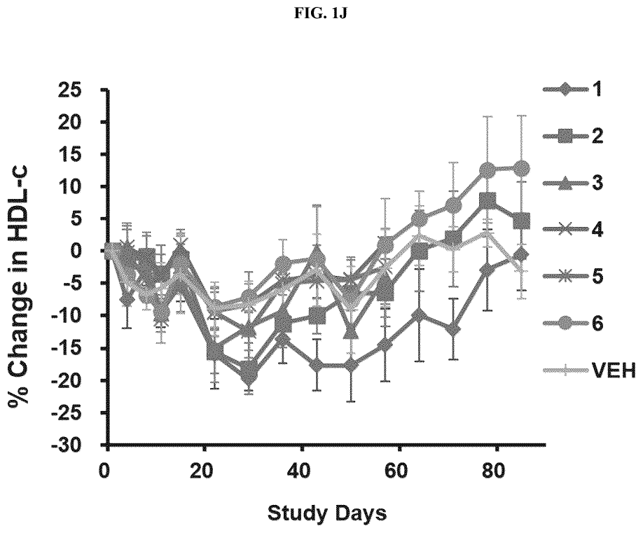

As illustrated in FIG. 1A to FIG. 1J, SRS-001 to SRS-005 were tested with SRS-006 as a control, and serum PCSK9 expression levels, LDL-c levels, total cholesterol levels, triglyceride levels, and HDL-c levels were monitored and quantified in comparison with the pre-dose level. Other clinical markers (e.g., body weight, food intake, total energy intake, clinical chemistry, complete blood count (CBC), animal training, cage-side observation, and clinical behavior check, etc.) were also monitored. Specifically, clinical chemistry was profiled with the followed parameters: GLU, INS, Cr, BUN, AST, ALT, ALB, BIL, Ca, PHOS, ALP, GGT, TP, CO2, CK, Na, K, CL, GLB, and A/G. CBC was performed for WBC, RBC, HGB, HCT, MCV, MCH, MCHC, PLT, RDW-CV, MPV, NEUT, NEUT %, LYMP, LYMP %, MONO, MONO %, EO, EO %, BASO, and BASO %. Exemplary items for the clinical behavior check included general appearance, respiration condition, urine condition, teeth condition, eyes condition, digestive system condition, genital and perineum condition, skin and pelage condition, behavior and neurological signs, oral cavity condition, mouth condition, ear condition, nose condition, intake condition, muscle condition, back condition, joint condition, bone condition, and extremities. A reduction of serum PCSK9 protein levels, LDL-c levels, and total cholesterol level were observed. Specifically, SRS-001 and SRS-002 effectively reduced PCSK9 protein levels, LDL-c levels, and total cholesterol level.

### Example 2—In Vitro Efficacy of siRNAs Targeting PCSK9

Primary non-human hepatocytes from BioreclamationIVT were thawed and plated on collagen-coated 96-well plates at a density of 5.4×10⁵ cells per well. Hepatocytes were treated with conjugated siRNAs in the absence of transfection reagents (free uptake). Cells were treated with siRNAs with a concentrations at 10 nM, 100 nM, or 500 nM. Cells were incubated at 37° C., 5% CO2 for 48 h. At the end of the incubation period, the cells were lysed, the mRNA was isolated, and the expression of the target gene was measured by qPCR and normalized to a house-keeping gene using standard protocols.

The modifications of the sense and antisense strands of the siRNAs tested are listed in Table 2. The in-vitro potency of the siRNAs are listed in Table 3.

TABLE 2
Sequences and Modifications of siRNAs Tested

| | | | | | | | | Antisense |
|---|---|---|---|---|---|---|---|---|
| | SEQ | | SEQ | Sense (5′→3′) | SEQ | | SEQ | (5′→3′) |
| Cmpd | ID | Sense (5′→3′) | ID | modified | ID | Antisense (5′→3′) | ID | modified |
| ID | No. | unmodified sequences | No. | sequences: | No. | unmodified sequences | No. | sequences |
| PCS | 25 | CCUCACCAAGAUC | 128 | cscsucacCaAGA | 231 | ACAUGCAGGAUCU | 334 | asCsaugcAggauC |
| K9- | | CUGCAUGU | | uccugcaugu | | UGGUGAGGUA | | uUgGugaggsusa |
| 1727- | | | | | | | | |
| 23 | | | | | | | | |
| PCS | 26 | CACCAAGAUCCUG | 129 | csasccaaGaUCC | 232 | AAGACAUGCAGGA | 335 | asAsgacaUgcagG |
| K9- | | CAUGUCUU | | ugcaugucuu | | UCUUGGUGAG | | aUcUuggugsasg |
| 1397- | | | | | | | | |
| 23 | | | | | | | | |
| PCS | 27 | CAAGAUCCUGCAU | 130 | csasagauCcUGC | 233 | UGGAAGACAUGCA | 336 | usGsgaagAcaugC |
| K9- | | GUCUUCCA | | augucuucca | | GGAUCUUGGU | | aGgAucuugsgsu |
| 1130- | | | | | | | | |
| 23 | | | | | | | | |
| PCS | 28 | UCCUGGCUUCCUG | 131 | uscscuggCuUC | 234 | AUCUUCACCAGGA | 337 | asUscuuc AccagG |
| K9- | | GUGAAGAU | | Cuggugaagau | | AGCCAGGAAG | | aAgCcaggasasg |
| 640- | | | | | | | | |
| 23 | | | | | | | | |
| PCS | 29 | CUGGCUUCCUGGU | 132 | csusggcuUcCU | 235 | UCAUCUUCACCAG | 338 | usCsaucuUcaccA |
| K9- | | GAAGAUGA | | Ggugaagauga | | GAAGCCAGGA | | gGaAgccagsgsa |
| 5061- | | | | | | | | |
| 23 | | | | | | | | |
| PCS | 30 | GGCUUCCUGGUGA | 133 | gsgscuucCuGG | 236 | ACUCAUCUUCACC | 339 | asCsucauCuucaC |
| K9- | | AGAUGAGU | | Ugaagaugagu | | AGGAAGCCAG | | cAgGaagccsasg |
| 1632- | | | | | | | | |
| 23 | | | | | | | | |
| PCS | 31 | GCUUCCUGGUGAA | 134 | gscsuuccUgGU | 237 | CACUCAUCUUCACC | 340 | csAscucaUcuucA |
| K9- | | GAUGAGUG | | Gaagaugagug | | AGGAAGCCA | | cCaGgaagcscsa |
| 4248- | | | | | | | | |
| 23 | | | | | | | | |
| PCS | 32 | UGGAGCUGGCCUU | 135 | usgsgagcUgGC | 238 | GCAACUUCAAGGC | 341 | gsCsaacuUcaagG |
| K9- | | GAAGUUGC | | Cuugaaguugc | | CAGCUCCAGC | | cCaGcuccasgsc |
| 3564- | | | | | | | | |
| 23 | | | | | | | | |
| PCS | 33 | AGUUGCCCCAUGU | 136 | asgsuugcCcCA | 239 | UGUAGUCGACAUG | 342 | usGsuaguCgacaU |
| K9- | | CGACUACA | | Ugucgacuaca | | GGGCAACUUC | | gGgGcaacususc |
| 3165- | | | | | | | | |
| 23 | | | | | | | | |
| PCS | 34 | UGCCCCAUGUCGA | 137 | usgsccccAuGU | 240 | CGAUGUAGUCGAC | 343 | csGsauguAgucgA |
| K9- | | CUACAUCG | | Cgacuacaucg | | AUGGGGCAAC | | cAuGgggcasasc |
| 1933- | | | | | | | | |
| 23 | | | | | | | | |
| PCS | 35 | CGGUACCGGGCGG | 138 | csgsguacCgGG | 241 | GUAUUCAUCCGCC | 344 | gsUsauucAuccgC |
| K9- | | AUGAAUAC | | Cggaugaauac | | CGGUACCGUG | | cCgGuaccgsusg |
| 4901- | | | | | | | | |
| 23 | | | | | | | | |
| PCS | 36 | GGUACCGGGCGGA | 139 | gsgsuaccGgGC | 242 | GGUAUUCAUCCGC | 345 | gsGsuauuCauccG |
| K9- | | UGAAUACC | | Ggaugaauacc | | CCGGUACCGU | | cCcGguaccsgsu |
| 1021- | | | | | | | | |
| 23 | | | | | | | | |
| PCS | 37 | GGCAGCCUGGUGG | 140 | gsgscagcCuGG | 243 | AUACACCUCCACCA | 346 | asUsacacCuccaCc |
| K9- | | AGGUGUAU | | Uggagguguau | | GGCUGCCUC | | AgGcugccsusc |
| 1977- | | | | | | | | |
| 23 | | | | | | | | |
| PCS | 38 | CAGCCUGGUGGAG | 141 | csasgccuGgUG | 244 | AGAUACACCUCCA | 347 | asGsauacAccucC |
| K9- | | GUGUAUCU | | Gagguguaucu | | CCAGGCUGCC | | aCcAggcugscsc |
| 727- | | | | | | | | |
| 23 | | | | | | | | |
| PCS | 39 | AGCCUGGUGGAGG | 142 | asgsccugGuGG | 245 | GAGAUACACCUCC | 348 | gsAsgauaCaccuC |
| K9- | | UGUAUCUC | | Agguguaucuc | | ACCAGGCUGC | | cAcCaggcusgsc |
| 2305- | | | | | | | | |
| 23 | | | | | | | | |
| PCS | 40 | GCCUGGUGGAGGU | 143 | gscscuggUgGA | 246 | GGAGAUACACCUC | 349 | gsGsagauAcaccU |
| K9- | | GUAUCUCC | | Gguguaucucc | | CACCAGGCUG | | cCaCcaggcsusg |
| 1330- | | | | | | | | |
| 23 | | | | | | | | |
| PCS | 41 | GUAUCUCCUAGAC | 144 | gsusaucuCcUA | 247 | AUGCUGGUGUCUA | 350 | asUsgcugGugucU |
| K9- | | ACCAGCAU | | Gacaccagcau | | GGAGAUACAC | | aGgAgauacsasc |
| 2942- | | | | | | | | |
| 23 | | | | | | | | |
| PCS | 42 | AUCUCCUAGACAC | 145 | asuscuccUaGAC | 248 | GUAUGCUGGUGUC | 351 | gsUsaugcUggugU |
| K9- | | CAGCAUAC | | accagcauac | | UAGGAGAUAC | | cUaGgagausasc |
| 3862- | | | | | | | | |
| 23 | | | | | | | | |
| PCS | 43 | UCCUAGACACCAG | 146 | uscscuagAcACC | 249 | UCUGUAUGCUGGU | 352 | usCsuguaUgcugG |
| K9- | | CAUACAGA | | agcauacaga | | GUCUAGGAGA | | uGuCuaggasgsa |
| 1225- | | | | | | | | |
| 23 | | | | | | | | |
| PCS | 44 | CUAGACACCAGCA | 147 | csusagacAcCAG | 250 | ACUCUGUAUGCUG | 353 | asCsucugUaugcU |
| K9- | | UACAGAGU | | cauacagagu | | GUGUCUAGGA | | gGuGucuagsgsa |
| 2328- | | | | | | | | |
| 23 | | | | | | | | |
| PCS | 45 | AUGGUCACCGACU | 148 | asusggucAcCG | 251 | AUUCUCGAAGUCG | 354 | asUsucucGaaguC |
| K9- | | UCGAGAAU | | Acuucgagaau | | GUGACCAUGA | | gGuGaccausgsa |
| 4494- | | | | | | | | |
| 23 | | | | | | | | |
| PCS | 46 | UCACCGACUUCGA | 149 | uscsaccgAcUUC | 252 | GCACAUUCUCGAA | 355 | gsCsacauUcucgA |
| K9- | | GAAUGUGC | | gagaaugugc | | GUCGGUGACC | | aGuCggugascsc |
| 2108- | | | | | | | | |
| 23 | | | | | | | | |
| PCS | 47 | CCUCAUAGGCCUG | 150 | cscsucauAgGCC | 253 | AUAAACUCCAGGC | 356 | asUsaaacUccagG |
| K9- | | GAGUUUAU | | uggaguuuau | | CUAUGAGGGU | | cCuAugaggsgsu |
| 1532- | | | | | | | | |
| 23 | | | | | | | | |
| PCS | 48 | GGCCUGGAGUUUA | 151 | gsgsccugGaGU | 254 | UUUCCGAAUAAAC | 357 | usUsuccgAauaaA |
| K9- | | UUCGGAAA | | Uuauucggaaa | | UCCAGGCCUA | | cUcCaggccsusa |
| 3158- | | | | | | | | |
| 23 | | | | | | | | |
| PCS | 49 | GCCUGGAGUUUAU | 152 | gscscuggAgUU | 255 | UUUUCCGAAUAAA | 358 | usUsuuccGaauaA |
| K9- | | UCGGAAAA | | Uauucggaaaa | | CUCCAGGCCU | | aCuCcaggcscsu |
| 2966- | | | | | | | | |
| 23 | | | | | | | | |
| PCS | 50 | UUGUGUCACAGAG | 153 | ususguguCaCA | 256 | AUGUCCCACUCUG | 359 | asUsguccCacucU |
| K9- | | UGGGACAU | | Gagugggacau | | UGACACAAAG | | gUgAcacaasasg |
| 3514- | | | | | | | | |
| 23 | | | | | | | | |
| PCS | 51 | CUGAUCCACUUCU | 154 | csusgaucCaCUU | 257 | UUUGGCAGAGAAG | 360 | usUsuggcAgagaA |
| K9- | | CUGCCAAA | | cucugccaaa | | UGGAUCAGUC | | gUgGaucagsusc |
| 3369- | | | | | | | | |
| 23 | | | | | | | | |
| PCS | 52 | AUCCACUUCUCUG | 155 | asusccacUuCUC | 258 | AUCUUUGGCAGAG | 361 | asUscuuuGgcagA |
| K9- | | CCAAAGAU | | ugccaaagau | | AAGUGGAUCA | | gAaGuggauscsa |
| 109- | | | | | | | | |
| 23 | | | | | | | | |
| PCS | 53 | UCUCUGCCAAAGA | 156 | uscsucugCcAA | 259 | UGAUGACAUCUUU | 362 | usGsaugaCaucuU |
| K9- | | UGUCAUCA | | Agaugucauca | | GGCAGAGAAG | | uGgCagagasasg |
| 821- | | | | | | | | |
| 23 | | | | | | | | |
| PCS | 54 | UGUCAUCAAUGAG | 157 | usgsucauCaAU | 260 | AACCAGGCCUCAU | 363 | asAsccagGccucA |
| K9- | | GCCUGGUU | | Gaggccugguu | | UGAUGACAUC | | uUgAugacasusc |
| 4228- | | | | | | | | |
| 23 | | | | | | | | |
| PCS | 55 | AGCUGUUUUGCAG | 158 | asgscuguUuUG | 261 | AUACAGUCCUGCA | 364 | asUsacagUccugC |
| K9- | | GACUGUAU | | Caggacuguau | | AAACAGCUGC | | aAaAcagcusgsc |
| 4399- | | | | | | | | |
| 23 | | | | | | | | |
| PCS | 56 | CUGUUUUGCAGGA | 159 | csusguuuUgCA | 262 | CCAUACAGUCCUG | 365 | csCsauacAguccU |
| K9- | | CUGUAUGG | | Ggacuguaugg | | CAAAACAGCU | | gCaAaacagscsu |
| 4785- | | | | | | | | |
| 23 | | | | | | | | |
| PCS | 57 | GGACUGUAUGGUC | 160 | gsgsacugUaUG | 263 | AGUGUGCUGACCA | 366 | asGsugugCugacC |
| K9- | | AGCACACU | | Gucagcacacu | | UACAGUCCUG | | aUaCaguccsusg |
| 1885- | | | | | | | | |
| 23 | | | | | | | | |
| PCS | 58 | ACUGUAUGGUCAG | 161 | ascsuguaUgGU | 264 | CGAGUGUGCUGAC | 367 | csGsagugUgcugA |
| K9- | | CACACUCG | | Cagcacacucg | | CAUACAGUCC | | cCaUacaguscsc |
| 3131- | | | | | | | | |
| 23 | | | | | | | | |
| PCS | 59 | CCCAGGUCUGGAA | 162 | cscscaggUcUG | 265 | ACUUUGCAUUCCA | 368 | asCsuuugCauucC |
| K9- | | UGCAAAGU | | Gaaugcaaagu | | GACCUGGGGC | | aGaCcugggsgsc |
| 3374- | | | | | | | | |
| 23 | | | | | | | | |
| PCS | 60 | UCUGGAAUGCAAA | 163 | uscsuggaAuGC | 266 | UCCUUGACUUUGC | 369 | usCscuugAcuuuG |
| K9- | | GUCAAGGA | | Aaagucaagga | | AUUCCAGACC | | cAuUccagascsc |
| 4465- | | | | | | | | |
| 23 | | | | | | | | |
| PCS | 61 | UAGACAACACGUG | 164 | usasgacaAcACG | 267 | UGACUACACACGU | 370 | usGsacuaCacacG |
| K9- | | UGUAGUCA | | uguguaguca | | GUUGUCUACG | | uGuUgucuascsg |
| 4027- | | | | | | | | |
| 23 | | | | | | | | |
| PCS | 62 | CUGGGGCUGAGCU | 165 | csusggggCuGA | 268 | CAUUUUAAAGCUC | 371 | csAsuuuuAaagcU |
| K9- | | UUAAAAUG | | Gcuuuaaaaug | | AGCCCCAGCC | | cAgCcccagscsc |
| 1571- | | | | | | | | |
| 23 | | | | | | | | |
| PCS | 63 | UGGGGCUGAGCUU | 166 | usgsgggcUgAG | 269 | CCAUUUUAAAGCU | 372 | csCsauuuUaaagC |
| K9- | | UAAAAUGG | | Cuuuaaaaugg | | CAGCCCCAGC | | uCaGccccasgsc |
| 1750- | | | | | | | | |
| 23 | | | | | | | | |
| PCS | 64 | GGGGCUGAGCUUU | 167 | gsgsggcuGaGC | 270 | ACCAUUUUAAAGC | 373 | asCscauuUuaaaG |
| K9- | | AAAAUGGU | | Uuuaaaauggu | | UCAGCCCCAG | | cUcAgccccsasg |
| 4079- | | | | | | | | |
| 23 | | | | | | | | |
| PCS | 65 | GGGCUGAGCUUUA | 168 | gsgsgcugAgCU | 271 | AACCAUUUUAAAG | 374 | asAsccauUuuaaA |
| K9- | | AAAUGGUU | | Uuaaaaugguu | | CUCAGCCCCA | | gCuCagcccscsa |
| 3172- | | | | | | | | |
| 23 | | | | | | | | |
| PCS | 66 | GGCUGAGCUUUAA | 169 | gsgscugaGcUU | 272 | GAACCAUUUUAAA | 375 | gsAsaccaUuuuaA |
| K9- | | AAUGGUUC | | Uaaaaugguuc | | GCUCAGCCCC | | aGcUcagccscsc |
| 4788- | | | | | | | | |
| 23 | | | | | | | | |
| PCS | 67 | AGCUUUAAAAUGG | 170 | asgscuuuAaAA | 273 | AGUCGGAACCAUU | 376 | asGsucggAaccaU |
| K9- | | UUCCGACU | | Ugguuccgacu | | UUAAAGCUCA | | uUuAaagcuscsa |
| 4139- | | | | | | | | |
| 23 | | | | | | | | |
| PCS | 68 | GCUUUAAAAUGGU | 171 | gscsuuuaAaAU | 274 | AAGUCGGAACCAU | 377 | asAsgucgGaaccA |
| K9- | | UCCGACUU | | Gguuccgacuu | | UUUAAAGCUC | | uUuUaaagcsusc |
| 5009- | | | | | | | | |
| 23 | | | | | | | | |
| PCS | 69 | UUUAAAAUGGUUC | 172 | ususuaaaAuGG | 275 | ACAAGUCGGAACC | 378 | asCsaaguCggaaC |
| K9- | | CGACUUGU | | Uuccgacuugu | | AUUUUAAAGC | | cAuUuuaaasgsc |
| 317- | | | | | | | | |
| 23 | | | | | | | | |
| PCS | 70 | UGCUGGCCUGGCC | 173 | usgscuggCcUG | 276 | CACUCAAGGGCCA | 379 | csAscucaAgggcC |
| K9- | | CUUGAGUG | | Gcccuugagug | | GGCCAGCAGC | | aGgCcagcasgsc |
| 3286- | | | | | | | | |
| 23 | | | | | | | | |
| PCS | 71 | GCUGGCCUGGCCC | 174 | gscsuggcCuGG | 277 | CCACUCAAGGGCC | 380 | csCsacuc AagggC |
| K9- | | UUGAGUGG | | Cccuugagugg | | AGGCCAGCAG | | cAgGccagcsasg |
| 2619- | | | | | | | | |
| 23 | | | | | | | | |
| PCS | 72 | CCCUCACUGUGGG | 175 | cscscucaCuGUG | 278 | UGAAAUGCCCCAC | 381 | usGsaaauGccccA |
| K9- | | GCAUUUCA | | gggcauuuca | | AGUGAGGGAG | | cAgUgagggsasg |
| 3186- | | | | | | | | |
| 23 | | | | | | | | |
| PCS | 73 | CACUGUGGGGCAU | 176 | csascuguGgGG | 279 | AUGGUGAAAUGCC | 382 | asUsggugAaaugC |
| K9- | | UUCACCAU | | Cauuucaccau | | CCACAGUGAG | | cCcAcagugsasg |
| 11-23 | | | | | | | | |
| PCS | 74 | CUGUGGGGCAUUU | 177 | csusguggGgCA | 280 | GAAUGGUGAAAUG | 383 | gsAsauggUgaaaU |
| K9- | | CACCAUUC | | Uuucaccauuc | | CCCCACAGUG | | gCcCcacagsusg |
| 840- | | | | | | | | |
| 23 | | | | | | | | |
| PCS | 75 | UGGGGCAUUUCAC | 178 | usgsgggcAuUU | 281 | UUUGAAUGGUGAA | 384 | usUsugaaUggugA |
| K9- | | CAUUCAAA | | Caccauucaaa | | AUGCCCCACA | | aAuGccccascsa |
| 404- | | | | | | | | |
| 23 | | | | | | | | |
| PCS | 76 | GCAUUUCACCAUU | 179 | gscsauuuCaCCA | 282 | CCUGUUUGAAUGG | 385 | csCsuguuUgaauG |
| K9- | | CAAACAGG | | uucaaacagg | | UGAAAUGCCC | | gUgAaaugcscsc |
| 26-23 | | | | | | | | |
| PCS | 77 | UUUAUUGAGCUCU | 180 | ususuauuGaGC | 283 | ACGGAACAAGAGC | 386 | asCsggaaCaagaG |
| K9- | | UGUUCCGU | | Ucuuguuccgu | | UCAAUAAAAG | | cUcAauaaasasg |
| 4467- | | | | | | | | |
| 23 | | | | | | | | |
| PCS | 78 | CCCUCAUCUCCAG | 181 | cscscucaUcUCC | 284 | ACAGUUAGCUGGA | 387 | asCsaguuAgcugG |
| K9- | | CUAACUGU | | agcuaacugu | | GAUGAGGGCC | | aGaUgagggscsc |
| 1934- | | | | | | | | |
| 23 | | | | | | | | |
| PCS | 79 | ACUGAGCCAGAAA | 182 | ascsugagCcAG | 285 | AAUCUGCGUUUCU | 388 | asAsucugCguuuC |
| K9- | | CGCAGAUU | | Aaacgcagauu | | GGCUCAGUUC | | uGgCucagususc |
| 1370- | | | | | | | | |
| 23 | | | | | | | | |
| PCS | 80 | UGAGCCAGAAACG | 183 | usgsagccAgAA | 286 | CCAAUCUGCGUUU | 389 | csCsaaucUgcguU |
| K9- | | CAGAUUGG | | Acgcagauugg | | CUGGCUCAGU | | uCuGgcucasgsu |
| 3338- | | | | | | | | |
| 23 | | | | | | | | |
| PCS | 81 | GAAGCCAAGCCUC | 184 | gsasagccAaGCC | 287 | AGUAAGAAGAGGC | 390 | asGsuaagAagagG |
| K9- | | UUCUUACU | | ucuucuuacu | | UUGGCUUCAG | | cUuGgcuucsasg |
| 4804- | | | | | | | | |
| 23 | | | | | | | | |
| PCS | 82 | AGCCAAGCCUCUU | 185 | asgsccaaGcCUC | 288 | GAAGUAAGAAGAG | 391 | gsAsaguaAgaagA |
| K9- | | CUUACUUC | | uuuuuu | | GCUUGGCUUC | | gGcUuggcususc |
| 1098- | | | | | | | | |
| 23 | | | | | | | | |
| PCS | 83 | CCCAAGCAAGCAG | 186 | cscscaagCaAGC | 289 | AUAAAUGUCUGCU | 392 | asUsaaauGucugC |
| K9- | | ACAUUUAU | | agacauuuau | | UGCUUGGGUG | | uUgCuugggsusg |
| 1350- | | | | | | | | |
| 23 | | | | | | | | |
| PCS | 84 | CCAAGCAAGCAGA | 187 | cscsaagcAaGCA | 290 | GAUAAAUGUCUGC | 393 | gsAsuaaaUgucuG |
| K9- | | CAUUUAUC | | gacauuuauc | | UUGCUUGGGU | | cUuGcuuggsgsu |
| 5305- | | | | | | | | |
| 23 | | | | | | | | |
| PCS | 85 | UUUUGGGUCUGUC | 188 | ususuuggGuCU | 291 | ACAGAGAGGACAG | 394 | asCsagagAggacA |
| K9- | | CUCUCUGU | | Guccucucugu | | ACCCAAAAGA | | gAcCcaaaasgsa |
| 478- | | | | | | | | |
| 23 | | | | | | | | |
| PCS | 86 | UCUGUCCUCUCUG | 189 | uscsugucCuCU | 292 | AAAGGCAACAGAG | 395 | asAsaggcAacagA |
| K9- | | UUGCCUUU | | Cuguugccuuu | | AGGACAGACC | | gAgGacagascsc |
| 4369- | | | | | | | | |
| 23 | | | | | | | | |
| PCS | 87 | CUGUCCUCUCUGU | 190 | csusguccUcUC | 293 | AAAAGGCAACAGA | 396 | asAsaaggCaacaG |
| K9- | | UGCCUUUU | | Uguugccuuuu | | GAGGACAGAC | | aGaGgacagsasc |
| 2723- | | | | | | | | |
| 23 | | | | | | | | |
| PCS | 88 | UGUCCUCUCUGUU | 191 | usgsuccuCuCU | 294 | AAAAAGGCAACAG | 397 | asAsaaagGcaacA |
| K9- | | GCCUUUUU | | Guugccuuuuu | | AGAGGACAGA | | gAgAggacasgsa |
| 1688- | | | | | | | | |
| 23 | | | | | | | | |
| PCS | 89 | GUCCUCUCUGUUG | 192 | gsusccucUcUG | 295 | UAAAAAGGCAACA | 398 | usAsaaaaGgcaaC |
| K9- | | CCUUUUUA | | Uugccuuuuua | | GAGAGGACAG | | aGaGaggacsasg |
| 84-23 | | | | | | | | |
| PCS | 90 | CCUCUCUGUUGCC | 193 | cscsucucUgUU | 296 | UGUAAAAAGGCAA | 399 | usGsuaaaAaggcA |
| K9- | | UUUUUACA | | Gccuuuuuaca | | CAGAGAGGAC | | aCaGagaggsasc |
| 3495- | | | | | | | | |
| 23 | | | | | | | | |
| PCS | 91 | GUUGCCUUUUUAC | 194 | gsusugccUuUU | 297 | AGUUGGCUGUAAA | 400 | asGsuuggCuguaA |
| K9- | | AGCCAACU | | Uacagccaacu | | AAGGCAACAG | | aAaGgcaacsasg |
| 2744- | | | | | | | | |
| 23 | | | | | | | | |
| PCS | 92 | UGCCUUUUUACAG | 195 | usgsccuuUuUA | 298 | AAAGUUGGCUGUA | 401 | asAsaguuGgcugU |
| K9- | | CCAACUUU | | Cagccaacuuu | | AAAAGGCAAC | | aAaAaggcasasc |
| 5036- | | | | | | | | |
| 23 | | | | | | | | |
| PCS | 93 | CCUUUUUACAGCC | 196 | cscsuuuuUaCA | 299 | GAAAAGUUGGCUG | 402 | gsAsaaagUuggcU |
| K9- | | AACUUUUC | | Gccaacuuuuc | | UAAAAAGGCA | | gUaAaaaggscsa |
| 1674- | | | | | | | | |
| 23 | | | | | | | | |
| PCS | 94 | UUUUACAGCCAAC | 197 | ususuuacAgCC | 300 | CUAGAAAAGUUGG | 403 | csUsagaaAaguuG |
| K9- | | UUUUCUAG | | Aacuuuucuag | | CUGUAAAAAG | | gCuGuaaaasasg |
| 4557- | | | | | | | | |
| 23 | | | | | | | | |
| PCS | 95 | GCCAACUUUUCUA | 198 | gscscaacUuUUC | 301 | AACAGGUCUAGAA | 404 | asAscaggUcuagA |
| K9- | | GACCUGUU | | uagaccuguu | | AAGUUGGCUG | | aAaGuuggcsusg |
| 1813- | | | | | | | | |
| 23 | | | | | | | | |
| PCS | 96 | CCAACUUUUCUAG | 199 | cscsaacuUuUCU | 302 | AAACAGGUCUAGA | 405 | asAsacagGucuaG |
| K9- | | ACCUGUUU | | agaccuguuu | | AAAGUUGGCU | | aAaAguuggscsu |
| 4497- | | | | | | | | |
| 23 | | | | | | | | |
| PCS | 97 | CAACUUUUCUAGA | 200 | csasacuuUuCU | 303 | AAAACAGGUCUAG | 406 | asAsaacaGgucuA |
| K9- | | CCUGUUUU | | Agaccuguuuu | | AAAAGUUGGC | | gAaAaguugsgsc |
| 2871- | | | | | | | | |
| 23 | | | | | | | | |
| PCS | 98 | ACUUUUCUAGACC | 201 | ascsuuuuCuAG | 304 | GCAAAACAGGUCU | 407 | gsCsaaaaCagguC |
| K9- | | UGUUUUGC | | Accuguuuugc | | AGAAAAGUUG | | uAgAaaagususg |
| 2000- | | | | | | | | |
| 23 | | | | | | | | |
| PCS | 99 | UUUUCUAGACCUG | 202 | ususuucuAgAC | 305 | AAGCAAAACAGGU | 408 | asAsgcaaAacagG |
| K9- | | UUUUGCUU | | Cuguuuugcuu | | CUAGAAAAGU | | uCuAgaaaasgsu |
| 47-23 | | | | | | | | |
| PCS | 100 | UUUCUAGACCUGU | 203 | ususucuaGaCC | 306 | AAAGCAAAACAGG | 409 | asAsagcaAaacaG |
| K9- | | UUUGCUUU | | Uguuuugcuuu | | UCUAGAAAAG | | gUcUagaaasasg |
| 1000- | | | | | | | | |
| 23 | | | | | | | | |
| PCS | 101 | UCUAGACCUGUUU | 204 | uscsuagaCcUG | 307 | CAAAAGCAAAACA | 410 | csAsaaagCaaaaCa |
| K9- | | UGCUUUUG | | Uuuugcuuuug | | GGUCUAGAAA | | GgUcuagasasa |
| 4329- | | | | | | | | |
| 23 | | | | | | | | |
| PCS | 102 | CUAGACCUGUUUU | 205 | csusagacCuGU | 308 | ACAAAAGCAAAAC | 411 | asCsaaaaGcaaaAc |
| K9- | | GCUUUUGU | | Uuugcuuugu | | AGGUCUAGAA | | AgGucuagsasa |
| 4826- | | | | | | | | |
| 23 | | | | | | | | |
| PCS | 103 | UAGACCUGUUUUG | 206 | usasgaccUgUU | 309 | UACAAAAGCAAAA | 412 | usAscaaaAgcaaA |
| K9- | | CUUUUGUA | | Uugcuuuugua | | CAGGUCUAGA | | aCaGgucuasgsa |
| 460- | | | | | | | | |
| 23 | | | | | | | | |
| PCS | 104 | AGACCUGUUUUGC | 207 | asgsaccuGuUU | 310 | UUACAAAAGCAAA | 413 | usUsacaaAagcaA |
| K9- | | UUUUGUAA | | Ugcuuuuguaa | | ACAGGUCUAG | | aAcAggucusasg |
| 2614- | | | | | | | | |
| 23 | | | | | | | | |
| PCS | 105 | GACCUGUUUUGCU | 208 | gsasccugUuUU | 311 | GUUACAAAAGCAA | 414 | gsUsuacaAaagcA |
| K9- | | UUUGUAAC | | Gcuuuuguaac | | AACAGGUCUA | | aAaCaggucsusa |
| 3284- | | | | | | | | |
| 23 | | | | | | | | |
| PCS | 106 | ACCUGUUUUGCUU | 209 | ascscuguUuUG | 312 | AGUUACAAAAGCA | 415 | asGsuuac AaaagC |
| K9- | | UUGUAACU | | Cuuuuguaacu | | AAACAGGUCU | | aAaAcagguscsu |
| 1766- | | | | | | | | |
| 23 | | | | | | | | |
| PCS | 107 | CUGUUUUGCUUUU | 210 | csusguuuUgCU | 313 | CAAGUUACAAAAG | 416 | csAsaguuAcaaaA |
| K9- | | GUAACUUG | | Uuuguaacuug | | CAAAACAGGU | | gCaAaacagsgsu |
| 3250- | | | | | | | | |
| 23 | | | | | | | | |
| PCS | 108 | GUUUUGCUUUUGU | 211 | gsusuuugCuUU | 314 | UUCAAGUUACAAA | 417 | usUscaagUuacaA |
| K9- | | AACUUGAA | | Uguaacuugaa | | AGCAAAACAG | | aAgCaaaacsasg |
| 2699- | | | | | | | | |
| 23 | | | | | | | | |
| PCS | 109 | UUUGCUUUUGUAA | 212 | ususugcuUuUG | 315 | UCUUCAAGUUACA | 418 | usCsuucaAguuaC |
| K9- | | CUUGAAGA | | Uaacuugaaga | | AAAGCAAAAC | | aAaAgcaaasasc |
| 2531- | | | | | | | | |
| 23 | | | | | | | | |
| PCS | 110 | UUGCUUUUGUAAC | 213 | ususgcuuUuGU | 316 | AUCUUCAAGUUAC | 419 | asUscuuc AaguuA |
| K9- | | UUGAAGAU | | Aacuugaagau | | AAAAGCAAAA | | cAaAagcaasasa |
| 1601- | | | | | | | | |
| 23 | | | | | | | | |
| PCS | 111 | CUUUUGUAACUUG | 214 | csusuuugUaAC | 317 | AAUAUCUUCAAGU | 420 | as Asuauc UucaaG |
| K9- | | AAGAUAUU | | Uugaagauauu | | UACAAAAGCA | | uUaCaaaagscsa |
| 1877- | | | | | | | | |
| 23 | | | | | | | | |
| PCS | 112 | UUUUGUAACUUGA | 215 | ususuuguAaCU | 318 | AAAUAUCUUCAAG | 421 | asAsauauCuucaA |
| K9- | | AGAUAUUU | | Ugaagauauuu | | UUACAAAAGC | | gUuAcaaaasgsc |
| 4491- | | | | | | | | |
| 23 | | | | | | | | |
| PCS | 113 | UUUGUAACUUGAA | 216 | ususuguaAcUU | 319 | UAAAUAUCUUCAA | 422 | usAsaauaUcuucA |
| K9- | | GAUAUUUA | | Gaagauauuua | | GUUACAAAAG | | aGuUacaaasasg |
| 3424- | | | | | | | | |
| 23 | | | | | | | | |
| PCS | 114 | UAACUUGAAGAUA | 217 | usasacuuGaAG | 320 | AGAAUAAAUAUCU | 423 | asGsaauaAauauC |
| K9- | | UUUAUUCU | | Auauuuauucu | | UCAAGUUACA | | uUcAaguuascsa |
| 2041- | | | | | | | | |
| 23 | | | | | | | | |
| PCS | 115 | AACUUGAAGAUAU | 218 | asascuugAaGA | 321 | CAGAAUAAAUAUC | 424 | csAsgaauAaauaU |
| K9- | | UUAUUCUG | | Uauuuauucug | | UUCAAGUUAC | | cUuCaaguusasc |
| 3270- | | | | | | | | |
| 23 | | | | | | | | |
| PCS | 116 | AAGAUAUUUAUUC | 219 | asasgauaUuUA | 322 | AAAACCCAGAAUA | 425 | asAsaaccCagaaUa |
| K9- | | UGGGUUUU | | Uucuggguuuu | | AAUAUCUUCA | | AaUaucuuscsa |
| 976- | | | | | | | | |
| 23 | | | | | | | | |
| PCS | 117 | AGAUAUUUAUUCU | 220 | asgsauauUuAU | 323 | CAAAACCCAGAAU | 426 | csAsaaacCcagaA |
| K9- | | GGGUUUUG | | Ucuggguuuug | | AAAUAUCUUC | | uAaAuaucususc |
| 1652- | | | | | | | | |
| 23 | | | | | | | | |
| PCS | 118 | AUAUUUAUUCUGG | 221 | asusauuu AuUC | 324 | UACAAAACCCAGA | 427 | usAscaaa AcccaG |
| K9- | | GUUUUGUA | | Uggguuuugua | | AUAAAUAUCU | | aAuAaauauscsu |
| 4408- | | | | | | | | |
| 23 | | | | | | | | |
| PCS | 119 | UAUUUAUUCUGGG | 222 | usasuuuaUuCU | 325 | CUACAAAACCCAG | 428 | csUsacaaAacccA |
| K9- | | UUUUGUAG | | Ggguuuuguag | | AAUAAAUAUC | | gAaUaaauasusc |
| 1023- | | | | | | | | |
| 23 | | | | | | | | |
| PCS | 120 | UUCUGGGUUUUGU | 223 | ususcuggGuUU | 326 | AAAAUGCUACAAA | 429 | asAsaaugCuacaA |
| K9- | | AGCAUUUU | | Uguagcauuuu | | ACCCAGAAUA | | aAcCcagaasusa |
| 998- | | | | | | | | |
| 23 | | | | | | | | |
| PCS | 121 | GGUUUUGUAGCAU | 224 | gsgsuuuuGuAG | 327 | UAAUAAAAAUGCU | 430 | usAsauaaAaaugC |
| K9- | | UUUUAUUA | | Cauuuuuauua | | ACAAAACCCA | | uAcAaaaccscsa |
| 2616- | | | | | | | | |
| 23 | | | | | | | | |
| PCS | 122 | GUUUUGUAGCAUU | 225 | gsusuuugUaGC | 328 | UUAAUAAAAAUGC | 431 | usUsaauaAaaauG |
| K9- | | UUUAUUAA | | Auuuuuauuaa | | UACAAAACCC | | cUaCaaaacscsc |
| 4780- | | | | | | | | |
| 23 | | | | | | | | |
| PCS | 123 | UUUUGUAGCAUUU | 226 | ususuuguAgCA | 329 | AUUAAUAAAAAUG | 432 | asUsuaauAaaaaU |
| K9- | | UUAUUAAU | | Uuuuuauuaau | | CUACAAAACC | | gCuAcaaaascsc |
| 1644- | | | | | | | | |
| 23 | | | | | | | | |
| PCS | 124 | AUUUUUAUUAAUA | 227 | asusuuuu AuUA | 330 | AGUCACCAUAUUA | 433 | asGsucacCauauU |
| K9- | | UGGUGACU | | Auauggugacu | | AUAAAAAUGC | | aAuAaaaausgsc |
| 4430- | | | | | | | | |
| 23 | | | | | | | | |
| PCS | 125 | UUUUUAUUAAUAU | 228 | ususuuuaUuAA | 331 | AAGUCACCAUAUU | 434 | asAsgucaCcauaU |
| K9- | | GGUGACUU | | Uauggugacuu | | AAUAAAAAUG | | uAaUaaaaasusg |
| 1274- | | | | | | | | |
| 23 | | | | | | | | |
| PCS | 126 | UUUUGCUUUUGUA | 229 | ususuugcUuUU | 332 | CUUCAAGUUACAA | 435 | csUsucaaGuuacA |
| K9- | | ACUUGAAG | | Guaacuugaag | | AAGCAAAACA | | aAaGcaaaascsa |
| 2790- | | | | | | | | |
| 23 | | | | | | | | |
| PCS | 127 | UUGUAGCAUUUUU | 230 | ususguagCaUU | 333 | AUAUUAAUAAAAA | 436 | asUsauuaAuaaaA |
| K9- | | AUUAAUAU | | Uuuauuaauau | | UGCUACAAAA | | aUgCuacaasasa |
| 5328- | | | | | | | | |
| 23 | | | | | | | | |

Notes (only applied to modified sequences in Table 2):
(s) = 3′-phosphorothioate; lower case = 2′ O-methyl modified nucleotide; uper case = 2′-fluoro modified nucleotide

TABLE 3
In Vitro Efficacy of siRNAs

| | | | | | | siRNA | |
|---|---|---|---|---|---|---|---|
| | | | | | | information | |
| | | | | | | position | |
| | | | | SEQ | | at | |
| | 500 | 100 | 10 | ID | Sequence context | human | |
| ID | nM | nM | nM | NO. | Sense (5′→3′) | transcripts | Region |
| PCSK9- | 39.4 | 48 | 9.1 | 437 | CCUCACCAAGAUCCUGC | 609 | CDS |
| 1727-23 | | | | | AUGU | | |
| PCSK9- | 38 | 38.7 | 38.3 | 438 | CACCAAGAUCCUGCAUG | 612 | CDS |
| 1397-23 | | | | | UCUU | | |
| PCSK9- | 34.2 | 36.1 | 26.8 | 439 | CAAGAUCCUGCAUGUCU | 615 | CDS |
| 1130-23 | | | | | UCCA | | |
| PCSK9-640- | 18.5 | 34.4 | 30.1 | 440 | UCCUGGCUUCCUGGUGA | 645 | CDS |
| 23 | | | | | AGAU | | |
| PCSK9- | 18.7 | 29.1 | 18.9 | 441 | CUGGCUUCCUGGUGAAG | 647 | CDS |
| 5061-23 | | | | | AUGA | | |
| PCSK9- | 24.6 | 31.2 | 34.8 | 442 | GGCUUCCUGGUGAAGAU | 649 | CDS |
| 1632-23 | | | | | GAGU | | |
| PCSK9- | 71.1 | 36.8 | 17.3 | 443 | GCUUCCUGGUGAAGAUG | 650 | CDS |
| 4248-23 | | | | | AGUG | | |
| PCSK9- | −21 | 1.9 | 15.3 | 444 | UGGAGCUGGCCUUGAAG | 680 | CDS |
| 3564-23 | | | | | UUGC | | |
| PCSK9- | 14.8 | 28.2 | 27.4 | 445 | AGUUGCCCCAUGUCGAC | 695 | CDS |
| 3165-23 | | | | | UACA | | |
| PCSK9- | 74.6 | 64.7 | 33.4 | 446 | UGCCCCAUGUCGACUAC | 698 | CDS |
| 1933-23 | | | | | AUCG | | |
| PCSK9- | 68.7 | 56.2 | 22.5 | 447 | CGGUACCGGGCGGAUGA | 781 | CDS |
| 4901-23 | | | | | AUAC | | |
| PCSK9- | 11.5 | 32.6 | 30.8 | 448 | GGUACCGGGCGGAUGAA | 782 | CDS |
| 1021-23 | | | | | UACC | | |
| PCSK9- | 19.5 | 24.9 | 13.5 | 449 | GGCAGCCUGGUGGAGGU | 817 | CDS |
| 1977-23 | | | | | GUAU | | |
| PCSK9-727- | 9.2 | 28.4 | 32.4 | 450 | CAGCCUGGUGGAGGUGU | 819 | CDS |
| 23 | | | | | AUCU | | |
| PCSK9- | 26 | 31.7 | 23.5 | 451 | AGCCUGGUGGAGGUGUA | 820 | CDS |
| 2305-23 | | | | | UCUC | | |
| PCSK9- | 28.8 | 38 | 17.3 | 452 | GCCUGGUGGAGGUGUAU | 821 | CDS |
| 1330-23 | | | | | CUCC | | |
| PCSK9- | 38.6 | 25.4 | 13.8 | 453 | GUAUCUCCUAGACACCA | 834 | CDS |
| 2942-23 | | | | | GCAU | | |
| PCSK9- | 0.4 | 20.2 | 21.9 | 454 | AUCUCCUAGACACCAGC | 836 | CDS |
| 3862-23 | | | | | AUAC | | |
| PCSK9- | 19.1 | 2.5 | −19.6 | 455 | UCCUAGACACCAGCAUA | 839 | CDS |
| 1225-23 | | | | | CAGA | | |
| PCSK9- | 54.9 | 41.2 | 17.1 | 456 | CUAGACACCAGCAUACA | 841 | CDS |
| 2328-23 | | | | | GAGU | | |
| PCSK9- | 44.2 | 31.1 | 31.2 | 457 | AUGGUCACCGACUUCGA | 889 | CDS |
| 4494-23 | | | | | GAAU | | |
| PCSK9- | 42.7 | 32.7 | 30.8 | 458 | UCACCGACUUCGAGAAU | 893 | CDS |
| 2108-23 | | | | | GUGC | | |
| PCSK9- | 35.9 | 35.7 | 22 | 459 | CCUCAUAGGCCUGGAGU | 1080 | CDS |
| 1532-23 | | | | | UUAU | | |
| PCSK9- | 33.6 | 42.2 | 32.7 | 460 | GGCCUGGAGUUUAUUCG | 1087 | CDS |
| 3158-23 | | | | | GAAA | | |
| PCSK9- | −54.6 | −45.4 | −20.7 | 461 | GCCUGGAGUUUAUUCGG | 1088 | CDS |
| 2966-23 | | | | | AAAA | | |
| PCSK9- | −9.1 | −23.5 | 0 | 462 | UUGUGUCACAGAGUGGG | 1424 | CDS |
| 3514-23 | | | | | ACAU | | |
| PCSK9- | 7.6 | 9.2 | −0.5 | 463 | CUGAUCCACUUCUCUGC | 1531 | CDS |
| 3369-23 | | | | | CAAA | | |
| PCSK9-109- | 3.1 | −3.4 | −0.4 | 464 | AUCCACUUCUCUGCCAA | 1534 | CDS |
| 23 | | | | | AGAU | | |
| PCSK9-821- | −12.2 | −20.43 | −17.5 | 465 | UCUCUGCCAAAGAUGUC | 1541 | CDS |
| 23 | | | | | AUCA | | |
| PCSK9- | −22.4 | −3 | −5.8 | 466 | UGUCAUCAAUGAGGCCU | 1554 | CDS |
| 4228-23 | | | | | GGUU | | |
| PCSK9- | 9.6 | 14.8 | 10.8 | 467 | AGCUGUUUUGCAGGACU | 1649 | CDS |
| 4399-23 | | | | | GUAU | | |
| PCSK9- | −4.7 | −29.7 | 10 | 468 | CUGUUUUGCAGGACUGU | 1651 | CDS |
| 4785-23 | | | | | AUGG | | |
| PCSK9- | −45.3 | 2.2 | −18.9 | 469 | GGACUGUAUGGUCAGCA | 1661 | CDS |
| 1885-23 | | | | | CACU | | |
| PCSK9- | −8.2 | 6.8 | 6.4 | 470 | ACUGUAUGGUCAGCACA | 1663 | CDS |
| 3131-23 | | | | | CUCG | | |
| PCSK9- | −14.7 | 5.8 | 11.3 | 471 | CCCAGGUCUGGAAUGCA | 2097 | CDS |
| 3374-23 | | | | | AAGU | | |
| PCSK9- | −8.3 | −5.1 | 15.9 | 472 | UCUGGAAUGCAAAGUCA | 2103 | CDS |
| 4465-23 | | | | | AGGA | | |
| PCSK9- | 17.5 | 14 | 9.1 | 473 | UAGACAACACGUGUGUA | 2237 | CDS |
| 4027-23 | | | | | GUCA | | |
| PCSK9- | −3.7 | 11.8 | 11.7 | 474 | CUGGGGCUGAGCUUUAA | 2408 | 3UTR |
| 1571-23 | | | | | AAUG | | |
| PCSK9- | −22.4 | 2.7 | 6.6 | 475 | UGGGGCUGAGCUUUAAA | 2409 | 3UTR |
| 1750-23 | | | | | AUGG | | |
| PCSK9- | −76.6 | −19.6 | −10 | 476 | GGGGCUGAGCUUUAAAA | 2410 | 3UTR |
| 4079-23 | | | | | UGGU | | |
| PCSK9- | −1.9 | 15 | −24.2 | 477 | GGGCUGAGCUUUAAAAU | 2411 | 3UTR |
| 3172-23 | | | | | GGUU | | |
| PCSK9- | 1.6 | 13 | 2.9 | 478 | GGCUGAGCUUUAAAAUG | 2412 | 3UTR |
| 4788-23 | | | | | GUUC | | |
| PCSK9- | 21.7 | 27.5 | 15.3 | 479 | AGCUUUAAAAUGGUUCC | 2417 | 3UTR |
| 4139-23 | | | | | GACU | | |
| PCSK9- | 19 | 15 | −2 | 480 | GCUUUAAAAUGGUUCCG | 2418 | 3UTR |
| 5009-23 | | | | | ACUU | | |
| PCSK9-317- | −2 | 16.9 | 3 | 481 | UUUAAAAUGGUUCCGAC | 2420 | 3UTR |
| 23 | | | | | UUGU | | |
| PCSK9- | 9.5 | 3.4 | −0.9 | 482 | UGCUGGCCUGGCCCUUG | 2503 | 3UTR |
| 3286-23 | | | | | AGUG | | |
| PCSK9- | 12.7 | 15.4 | 5.2 | 483 | GCUGGCCUGGCCCUUGA | 2504 | 3UTR |
| 2619-23 | | | | | GUGG | | |
| PCSK9- | −0.14 | 22 | 4.4 | 484 | CCCUCACUGUGGGGCAU | 2597 | 3UTR |
| 3186-23 | | | | | UUCA | | |
| PCSK9-11- | 37.9 | 43.2 | 30.1 | 485 | CACUGUGGGGCAUUUCA | 2601 | 3UTR |
| 23 | | | | | CCAU | | |
| PCSK9-840- | 28.5 | 17.8 | 1.4 | 486 | CUGUGGGGCAUUUCACC | 2603 | 3UTR |
| 23 | | | | | AUUC | | |
| PCSK9-404- | 7.6 | 9.8 | −24.7 | 487 | UGGGGCAUUUCACCAUU | 2606 | 3UTR |
| 23 | | | | | CAAA | | |
| PCSK9-26- | −3.8 | 11.5 | 13.3 | 488 | GCAUUUCACCAUUCAAA | 2610 | 3UTR |
| 23 | | | | | CAGG | | |
| PCSK9- | 46.6 | 42.8 | 10.6 | 489 | UUUAUUGAGCUCUUGUU | 2694 | 3UTR |
| 4467-23 | | | | | CCGU | | |
| PCSK9- | 28.6 | 26.6 | 6.7 | 490 | CCCUCAUCUCCAGCUAA | 2838 | 3UTR |
| 1934-23 | | | | | CUGU | | |
| PCSK9- | 36.3 | 38.1 | 5 | 491 | ACUGAGCCAGAAACGCA | 3196 | 3UTR |
| 1370-23 | | | | | GAUU | | |
| PCSK9- | 35 | 36.6 | 20 | 492 | UGAGCCAGAAACGCAGA | 3198 | 3UTR |
| 3338-23 | | | | | UUGG | | |
| PCSK9- | 13.8 | 19.9 | 27.2 | 493 | GAAGCCAAGCCUCUUCU | 3228 | 3UTR |
| 4804-23 | | | | | UACU | | |
| PCSK9- | 34.9 | 32.4 | 20.8 | 494 | AGCCAAGCCUCUUCUUA | 3230 | 3UTR |
| 1098-23 | | | | | CUUC | | |
| PCSK9- | 23.2 | 40.8 | 29.8 | 495 | CCCAAGCAAGCAGACAU | 3464 | 3UTR |
| 1350-23 | | | | | UUAU | | |
| PCSK9- | −19.6 | 13.9 | 5.8 | 496 | CCAAGCAAGCAGACAUU | 3465 | 3UTR |
| 5305-23 | | | | | UAUC | | |
| PCSK9-478- | 21 | −0.4 | 7.2 | 497 | UUUUGGGUCUGUCCUCU | 3486 | 3UTR |
| 23 | | | | | CUGU | | |
| PCSK9- | 47.8 | 34.6 | 28.9 | 498 | UCUGUCCUCUCUGUUGC | 3493 | 3UTR |
| 4369-23 | | | | | CUUU | | |
| PCSK9- | 32.2 | 44.3 | 41.9 | 499 | CUGUCCUCUCUGUUGCC | 3494 | 3UTR |
| 2723-23 | | | | | UUUU | | |
| PCSK9- | 47.1 | 50.7 | 46.2 | 500 | UGUCCUCUCUGUUGCCU | 3495 | 3UTR |
| 1688-23 | | | | | UUUU | | |
| PCSK9-84- | 23.5 | 34.6 | 28.9 | 501 | GUCCUCUCUGUUGCCUU | 3496 | 3UTR |
| 23 | | | | | UUUA | | |
| PCSK9- | 21 | 24.9 | 35 | 502 | CCUCUCUGUUGCCUUUU | 3498 | 3UTR |
| 3495-23 | | | | | UACA | | |
| PCSK9- | 29.1 | 41.5 | 45 | 503 | GUUGCCUUUUUACAGCC | 3505 | 3UTR |
| 2744-23 | | | | | AACU | | |
| PCSK9- | 51.1 | 43.7 | 20.4 | 504 | UGCCUUUUUACAGCCAA | 3507 | 3UTR |
| 5036-23 | | | | | CUUU | | |
| PCSK9- | 47.2 | 23.2 | −10.5 | 505 | CCUUUUUACAGCCAACU | 3509 | 3UTR |
| 1674-23 | | | | | UUUC | | |
| PCSK9- | 47.5 | 39.4 | 4.7 | 506 | UUUUACAGCCAACUUUU | 3512 | 3UTR |
| 4557-23 | | | | | CUAG | | |
| PCSK9- | 53.2 | 49.6 | 26.2 | 507 | GCCAACUUUUCUAGACC | 3519 | 3UTR |
| 1813-23 | | | | | UGUU | | |
| PCSK9- | 57.2 | 59.5 | 37.2 | 508 | CCAACUUUUCUAGACCU | 3520 | 3UTR |
| 4497-23 | | | | | GUUU | | |
| PCSK9- | 50.7 | 51.1 | 31.4 | 509 | CAACUUUUCUAGACCUG | 3521 | 3UTR |
| 2871-23 | | | | | UUUU | | |
| PCSK9- | 20.8 | 28.7 | 25 | 510 | ACUUUUCUAGACCUGUU | 3523 | 3UTR |
| 2000-23 | | | | | UUGC | | |
| PCSK9-47- | 44.1 | 44.8 | 32.1 | 511 | UUUUCUAGACCUGUUUU | 3525 | 3UTR |
| 23 | | | | | GCUU | | |
| PCSK9- | 62.7 | 54.8 | 31.4 | 512 | UUUCUAGACCUGUUUUG | 3526 | 3UTR |
| 1000-23 | | | | | CUUU | | |
| PCSK9- | 26.1 | 9.8 | 14 | 513 | UCUAGACCUGUUUUGCU | 3528 | 3UTR |
| 4329-23 | | | | | UUUG | | |
| PCSK9- | 48.4 | 35.9 | 17.4 | 514 | CUAGACCUGUUUUGCUU | 3529 | 3UTR |
| 4826-23 | | | | | UUGU | | |
| PCSK9-460- | 28.8 | 29.2 | 20 | 515 | UAGACCUGUUUUGCUUU | 3530 | 3UTR |
| 23 | | | | | UGUA | | |
| PCSK9- | 52.4 | 27.8 | 31 | 516 | AGACCUGUUUUGCUUUU | 3531 | 3UTR |
| 2614-23 | | | | | GUAA | | |
| PCSK9- | 11.1 | 1.9 | 11.9 | 517 | GACCUGUUUUGCUUUUG | 3532 | 3UTR |
| 3284-23 | | | | | UAAC | | |
| PCSK9- | 43.8 | 17 | 6 | 518 | ACCUGUUUUGCUUUUGU | 3533 | 3UTR |
| 1766-23 | | | | | AACU | | |
| PCSK9- | 32.9 | 31.1 | 13.3 | 519 | CUGUUUUGCUUUUGUAA | 3535 | 3UTR |
| 3250-23 | | | | | CUUG | | |
| PCSK9- | 47.7 | 29 | 20.6 | 520 | GUUUUGCUUUUGUAACU | 3537 | 3UTR |
| 2699-23 | | | | | UGAA | | |
| PCSK9- | 8.9 | 5.4 | −0.8 | 521 | UUUUGCUUUUGUAACUU | 3538 | 3UTR |
| 2790-23 | | | | | GAAG | | |
| PCSK9- | 22.4 | 15.1 | −18.8 | 522 | UUUGCUUUUGUAACUUG | 3539 | 3UTR |
| 2531-23 | | | | | AAGA | | |
| PCSK9- | 48.1 | 45 | 22.4 | 523 | UUGCUUUUGUAACUUGA | 3540 | 3UTR |
| 1601-23 | | | | | AGAU | | |
| PCSK9- | 65.6 | 64.2 | 37.4 | 524 | CUUUUGUAACUUGAAGA | 3543 | 3UTR |
| 1877-23 | | | | | UAUU | | |
| PCSK9- | 58 | 59 | 35.2 | 525 | UUUUGUAACUUGAAGAU | 3544 | 3UTR |
| 4491-23 | | | | | AUUU | | |
| PCSK9- | 58.4 | 50.8 | 29.7 | 526 | UUUGUAACUUGAAGAUA | 3545 | 3UTR |
| 3424-23 | | | | | UUUA | | |
| PCSK9- | 62.2 | 58.7 | 28.4 | 527 | UAACUUGAAGAUAUUUA | 3549 | 3UTR |
| 2041-23 | | | | | UUCU | | |
| PCSK9- | 42.7 | 36.9 | 33.7 | 528 | AACUUGAAGAUAUUUAU | 3550 | 3UTR |
| 3270-23 | | | | | UCUG | | |
| PCSK9-976- | 40 | 44 | 23.5 | 529 | AAGAUAUUUAUUCUGGG | 3556 | 3UTR |
| 23 | | | | | UUUU | | |
| PCSK9- | 18 | 1.1 | −21.9 | 530 | AGAUAUUUAUUCUGGGU | 3557 | 3UTR |
| 1652-23 | | | | | UUUG | | |
| PCSK9- | 14.9 | 23.6 | 18 | 531 | AUAUUUAUUCUGGGUUU | 3559 | 3UTR |
| 4408-23 | | | | | UGUA | | |
| PCSK9- | 22.9 | 25.6 | 12.5 | 532 | UAUUUAUUCUGGGUUUU | 3560 | 3UTR |
| 1023-23 | | | | | GUAG | | |
| PCSK9-998- | 55.3 | 51.1 | 35.3 | 533 | UUCUGGGUUUUGUAGCA | 3566 | 3UTR |
| 23 | | | | | UUUU | | |
| PCSK9- | 32.8 | 43.9 | 24.2 | 534 | GGUUUUGUAGCAUUUUU | 3571 | 3UTR |
| 2616-23 | | | | | AUUA | | |
| PCSK9- | 38.9 | 41 | 36.2 | 535 | GUUUUGUAGCAUUUUUA | 3572 | 3UTR |
| 4780-23 | | | | | UUAA | | |
| PCSK9- | 31.5 | 37.2 | 31.7 | 536 | UUUUGUAGCAUUUUUAU | 3573 | 3UTR |
| 1644-23 | | | | | UAAU | | |
| PCSK9- | 24 | 9.6 | 26.7 | 537 | UUGUAGCAUUUUUAUUA | 3575 | 3UTR |
| 5328-23 | | | | | AUAU | | |
| PCSK9- | 28 | 30.1 | 19.8 | 538 | AUUUUUAUUAAUAUGGU | 3582 | 3UTR |
| 4430-23 | | | | | GACU | | |
| PCSK9- | 13.8 | 29.5 | −1.2 | 539 | UUUUUAUUAAUAUGGUG | 3583 | 3UTR |
| 1274-23 | | | | | ACUU | | |

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A polynucleic acid molecule for modulating expression of proprotein convertase subtilisin/kexin type 9 serine protease (PCSK9) gene, wherein the polynucleic acid molecule comprises at least 16, 17, or 18 consecutive nucleotides that are complementary to a sequence selected from SEQ ID NOs: 1, 3, 5, 7, and 9 with no more than 1, 2, 3, 4 mismatches.

2. A polynucleic acid molecule for modulating expression of proprotein convertase subtilisin/kexin type 9 serine protease (PCSK9) gene, wherein the polynucleic acid molecule comprises a sequence that is at least 85%, at least 90%, at least 95% complementary to a sequence selected from SEQ ID NOs: 1, 3, 5, 7, and 9.

3. The polynucleic acid molecule of claim 1 or claim 2, wherein the polynucleic acid molecule is a double-stranded nucleic acid molecule comprising a sense strand and an antisense strand.

4. The polynucleic acid molecule of claim 3, wherein the sense strand comprises at least 85%, at least 90%, at least 95% identical to a sequence selected from SEQ ID NOs: 1, 3, 5, 7, and 9.

5. The polynucleic acid molecule of claim 3, wherein the antisense strand comprises at least 85%, at least 90%, at least 95% identical to a sequence selected from SEQ ID NOs: 2, 4, 6, 8, and 10.

6. The polynucleic acid molecule of one of claims 3-5, wherein the sense strand comprises a nucleic acid sequence comprising at least 16, 17, 18, 19, or 20 consecutive nucleotides of a sequence selected from SEQ ID NOs: 1, 3, 5, 7, and 9, with no more than 1, 2, 3, or 4 mismatches.

7. The polynucleic acid molecule of any one of claims 3-6, wherein the antisense strand comprises a nucleic acid sequence comprising at least 16, 17, 18, 19, or 20 consecutive nucleotides of a sequence selected from SEQ ID NOs: 2, 4, 6, 8, and 10, with no more than 1, 2, 3, or 4 mismatches

8. The polynucleic acid molecule of any one of claims 3-7, wherein the sense strand comprises one of SEQ ID NOs: 1, 3, 5, 7, 9, and the antisense strand comprises one of SEQ ID NOs: 2, 4, 6, 8, 10.

9. The polynucleic acid molecule of any one of claims 1-8, wherein the polynucleic acid molecule comprises (1) a 2′-fluoro modified nucleotides; (2) a 2′-O-methyl modified nucleotides; or (3) a modified internucleotide linkage.

10. The polynucleic acid molecule of any one of claims 1-9, wherein the polynucleic acid molecule comprises at least two consecutive modified internucleotide linkages at the 5′ end.

11. The polynucleic acid molecule of any one of claims 1-9, wherein the polynucleic acid molecule comprises at least two internucleotide linkages among three internucleotide linkages at the 3′end substituted with modified internucleotide linkages.

12. The polynucleic acid molecule of any one of claims 1-11, wherein the sense strand comprises ‘5-NfsnsNfnNfnNfnNfNfNfnNfnNfnNfnNfnNf-3’, wherein the antisense strand comprises ‘5-nsNfsnNfnNfnNfnNfnnnNfnNfnNfnNfnsnsn-3’, wherein “Nf” stands for a 2′-fluoro modified nucleotide, “n” stands for a 2′-O-methyl modified nucleotide, “s” stands for a 3′-phosphorothioate.

13. The polynucleic acid molecule of any one of claims 1-11, wherein the sense strand comprises ‘5-nsnsnnnnNfnNfNfNfnnnnnnnnnn-3’, wherein the antisense strand comprises ‘5-nsNfsnnnNfnNfNfnnnnNfnNfnnnnnsnsn-3’, wherein “Nf” stands for a 2′-fluoro modified nucleotide, “n” stands for a 2′-O-methyl modified nucleotide, “s” stands for a 3′-phosphorothioate.

14. The polynucleic acid molecule of any one of claims 1-11, wherein the sense strand comprises ‘5-nsnsnnnnnnNfnNfnnnnnnnnnn-3’, wherein the antisense strand comprises ‘5-nsNfsnnnnnnnnnNfnNfnnnnnnnsnsn-3’, wherein “Nf” stands for a 2′-fluoro modified nucleotide, “n” stands for a 2′-O-methyl modified nucleotide, “s” stands for a 3′-phosphorothioate.

15. The polynucleic acid molecule of any one of claims 1-11, wherein the sense strand comprises ‘5-nsnsnnnnNfnNfnNfnnnnnnnnnn-3’, wherein the antisense strand comprises ‘5-nsNfsnnnnnnnnnNfnNfnNfnnnnnsnsn-3’, wherein “Nf” stands for a 2′-fluoro modified nucleotide, “n” stands for a 2′-O-methyl modified nucleotide, “s” stands for a 3′-phosphorothioate.

16. The polynucleic acid molecule of any one of claims 9-15, wherein the modified internucleotide linkage is a phosphorothioate linkage.

17. The polynucleic acid molecule of claim 16, wherein the phosphorothioate linkage a stereochemically enriched phosphorothioate internucleotide linkage.

18. The polynucleic acid molecule of claim 17, wherein the stereochemically enriched phosphorothioate internucleotide linkage is an SP chiral internucleotide phosphorothioate linkage.

19. The polynucleic acid molecule of one of claim 9-15, wherein the polynucleic acid comprises a plurality of modified internucleotide linkages, and at least 1, 2, 3, or 4 of the plurality of modified internucleotide linkages are stereochemically enriched phosphorothioate internucleotide linkages.

20. The polynucleic acid molecule of claim 19, wherein the stereochemically enriched phosphorothioate internucleotide linkages comprise both R- and S-isomers.

21. The polynucleic acid molecule of one of claims 17-20, wherein the stereochemically enriched phosphorothioate internucleotide linkage(s) is disposed between two consecutive nucleosides that are two of six 5′ or 3′-terminal nucleosides of the sense strand or the antisense strand.

22. The polynucleic acid molecule of any one of claims 1-21, wherein the polynucleic acid molecule comprises a hypoxanthine nucleobase-containing nucleoside substitution.

23. The polynucleic acid molecule of claim 22, wherein the hypoxanthine nucleobase-containing nucleoside substitution is an inosine substitution.

24. The polynucleic acid molecule of claim 23, wherein the inosine substitution is within a seed region of the antisense strand.

25. The polynucleic acid molecule of claim 23, wherein the inosine substitution is within 7 nucleotides from the 5′ end of the antisense strand.

26. The polynucleic acid molecule of any one of claims 1-25, wherein the polynucleic acid molecule comprises an abasic substitution.

27. The polynucleic acid molecule of claim 26, wherein the abasic substitution is at the 5^{th} or 7^{th} nucleotide from the 5′ end.

28. The polynucleic acid molecule of any one of claims 9-27, wherein the cytotoxicity of the polynucleic acid molecule is decreased compared to unmodified polynucleic acid.

29. The polynucleic acid molecule of any one of claims 9-28, wherein the sense strand comprises a nucleic acid sequence that is at least 80%, at least 85%, at least 90%, at least 95% identical to a sequence selected from SEQ ID NOs: 13, 15, 17, 19, and 21.

30. The polynucleic acid molecule of any one of claims 9-28, wherein the sense strand comprises a nucleic acid sequence comprising at least 15, 16, 17, 18, 19, or 20 consecutive nucleotides of a sequence selected from SEQ ID NOs: 13, 15, 17, 19, and 21, with no more than 1, 2, 3, or 4 mismatches.

31. The polynucleic acid molecule of any one of claims 9-30, wherein the antisense strand comprises a nucleic acid sequence that is at least 85%, at least 90%, at least 95% identical to a sequence selected from SEQ ID NOs: 14, 16, 18, 20, and 22.

32. The polynucleic acid molecule of any one of claims 9-30, wherein the antisense strand comprises a nucleic acid sequence comprising at least 15, 16, 17, 18, 19, or 20 consecutive nucleotides of a sequence selected from SEQ ID NOs: 14, 16, 18, 20, and 22 with no more than 1, 2, 3, or 4 mismatches.

33. The polynucleic acid molecule of any one of claims 9-32, wherein the sense strand comprises a sequence selected from a nucleic acid sequence of SEQ ID NOs: 13, 15, 17, 19, and 21, and the antisense strand comprises a sequence selected from a nucleic acid sequence of SEQ ID NOs: 14, 16, 18, 20, and 22.

34. The polynucleic acid molecule of claim 1, wherein the polynucleic acid molecule is a single-stranded nucleic acid molecule.

35. The polynucleic acid molecule of any one of claims 1-34, wherein the polynucleic acid molecule is 16-30 base pairs in length.

36. The polynucleic acid molecule of any one of claims 1-34, wherein the polynucleic acid molecule is 19-25, or 21-23 base pairs in length.

37. A polynucleic acid molecule for modulating expression of proprotein convertase subtilisin/kexin type 9 serine protease (PCSK9) gene, wherein polynucleic acid molecule comprises:
(a) an antisense strand comprising the nucleotide sequence of usUfsacaaaagcaAfaAfcAfggucusasg (SEQ ID NO:14) and a sense strand comprising the nucleotide sequence of asgsaccuGfuUfuUfgcuuuuguaa (SEQ ID NO:13); or
(b) an antisense strand comprising the nucleotide sequence of usUfsucaaguuacAfaAfaGfcaaaascsa (SEQ ID NO:16) and a sense strand comprising the nucleotide sequence of ususuugcUfuUfuGfuaacuugaaa (SEQ ID NO:15);
wherein smaller case “n” stands for 2′-O-methyl modified nucleotide, upper case followed with an “f” (i.e., “Nf”) stands for 2′-fluoro modified nucleotide, and “s” stands for 3′-phosphorothioate.

38. A polynucleic acid molecule conjugate for modulating expression of proprotein convertase subtilisin/kexin type 9 serine protease (PCSK9) gene, wherein the polynucleic acid molecule conjugate comprises a polynucleic acid molecule of any one of claims 1-37 and an asialoglycoprotein receptor targeting moiety.

39. The polynucleic acid molecule conjugate of claim 38, wherein the polynucleic acid molecule and the asialoglycoprotein receptor targeting moiety is coupled via a linker.

40. The polynucleic acid molecule conjugate of claim 39, wherein the linker comprises formula (IV) below, 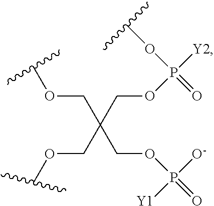
wherein at least one of Y1 and Y2 is a nucleotide in the polynucleic acid molecule.

41. The polynucleic acid molecule conjugate of claim 40, wherein the Y1 is the last nucleotide on the 3′-terminus of the sense strand of the polynucleic acid molecule.

42. The polynucleic acid molecule conjugate of claim 40, wherein the Y1 and Y2 are two consecutive nucleotides in the polynucleic acid molecule.

43. The polynucleic acid molecule conjugate of any one of claims 38-42, wherein the asialoglycoprotein receptor targeting moiety comprises N-Acetylgalactosamine (GalNAc).

44. The polynucleic acid molecule conjugate of any one of claims 39-43, wherein the linker and the asialoglycoprotein receptor targeting moiety with the last nucleotide on the 3′-terminus of the sense strand of the polynucleic acid molecule are shown in Formula (V′): 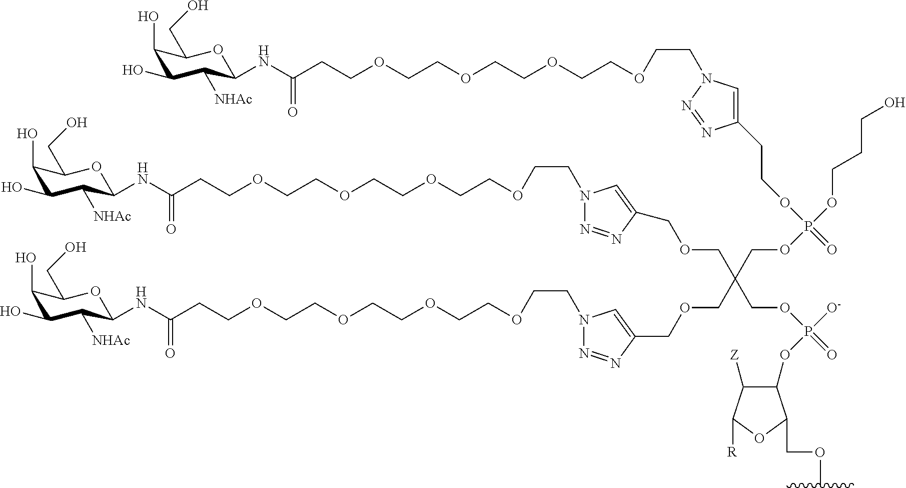
(V′), wherein Z in formula (V′) is —H, —OH, —O-Methyl, —F, or —O-methoxyethyl, and R in formula (V′) is adenine, uracil, guanine, cytosine, thymine, abasic, or others.

45. A pharmaceutical composition comprising a polynucleic acid molecule of any one of claims 1-37 or a polynucleic acid molecule conjugate of any one of claims 38-44, and a pharmaceutically acceptable excipient.

46. The pharmaceutical composition of claim 45, wherein the pharmaceutical composition is formulated as a nanoparticle formulation.

47. The pharmaceutical composition of claim 45 or claim 46, wherein the pharmaceutical composition is formulated for parenteral, oral, intranasal, buccal, rectal, transdermal, intravenous, subcutaneous, or intrathecal administration.

48. A method of modulating expression of proprotein convertase subtilisin/kexin type 9 serine protease (PCSK9) gene in a subject, comprising: administering to the subject a polynucleic acid molecule of any one of claims 1-37, a polynucleic acid molecule conjugate of any one of claims 38-44, or a pharmaceutical composition of claims 45-47, thereby modulating the expression of PCSK9 gene in the subject.

49. A method of modulating low-density lipoproteins (LDL) in a subject in need thereof, comprising: administering to the subject a polynucleic acid molecule of any one of claims 1-37, a polynucleic acid molecule conjugate of any one of claims 38-44, or a pharmaceutical composition of claims 45-47, wherein the polynucleic acid molecule of any one of claims 1-37, a polynucleic acid molecule conjugate of any one of claims 38-44, or a pharmaceutical composition of claims 45-47 reduces the expression of PCSK9 gene in the subject.

50. The method of claim 49, wherein the subject in need thereof suffers from hypercholesterolemia, familial hypercholesterolemia, or other high cholesterol-associated diseases.

51. A method of modulating cholesterol in a subject in need thereof, comprising: administering to the subject a polynucleic acid molecule of any one of claims 1-37, a polynucleic acid molecule conjugate of any one of claims 38-44, or a pharmaceutical composition of claims 45-47, wherein the polynucleic acid molecule of any one of claims 1-37, a polynucleic acid molecule conjugate of any one of claims 38-44, or a pharmaceutical composition of claims 45-47 reduces the expression of PCSK9 gene in the subject.

52. The method of claim 51, wherein the subject in need thereof suffers from hypercholesterolemia, familial hypercholesterolemia, or other high cholesterol-associated diseases.
